(19) United States
(12) Patent Application Publication
(10) Pub. No.: US 2024/0083998 A1
(43) Pub. Date: Mar. 14, 2024

(54) PREVENTION AND TREATMENT OF STEROID-RESISTANT OR GUT GRAFT-VERSUS-HOST DISEASE (GVHD)

(71) Applicant: CIY OF HOPE, Duarte, CA (US)

(72) Inventors: Defu ZENG, Duarte, CA (US); Qingxiao SONG, Duarte, CA (US); Arthur D. RIGGS, Duarte, CA (US)

(73) Assignee: CITY OF HOPE, Duarte, CA (US)

(21) Appl. No.: 18/270,706

(22) PCT Filed: Jan. 3, 2022
(86) PCT No.: PCT/US22/11051

§ 371 Date: Jun. 30, 2023

Related U.S. Application Data
(60) Provisional application No. 63/133,703, filed on Jan. 4, 2021.

(51) Int. Cl. C07K 16/24; A61K 35/15; A61K 35/17; A61P 1/00; A61P 37/06; C07K 16/30; C07K 16/40

(52) U.S. Cl. CPC C07K 16/248; A61K 35/15; A61K 35/17; A61P 1/00; A61P 37/06; C07K 16/3007; C07K 16/40; A61K 2039/505

(57) ABSTRACT

Disclosed is a method of preventing or treating acute GVHD (aGVHD) such as gut aGVHD, steroid-resistant aGVHD, and steroid-resistant gut aGVHD in a subject receiving a hematopoietic cell transplantation (HCT) or autoimmune colitis by administering to the subject an effective amount of an anti-IL-22 antibody, an anti-IL-6 antibody, donor-type CX3CR1^{hi} MNPs, donor-type NK cells, a ceacam-1 antagonist, an anti-Gr-1 antibody, or a combination thereof.

### PRIORITY CLAIM

This application claims priority to U.S. Provisional Patent Application No. 63/133,703, filed Jan. 4, 2021, the contents of which are hereby incorporated by reference in its entirety.

### STATEMENT REGARDING FEDERALLY SPONSORED RESEARCH

This invention was made with government support under Grant Numbers R01 Al066008 and R01 CA228465, awarded by the National Institutes of Health. The government has certain rights in the invention.

### SEQUENCE LISTING

This application contains a Sequence Listing, which was submitted in ASCII format via EFS-Web, and is hereby incorporated by reference in its entirety. The ASCII copy, created on Jan. 3, 2022, is named SequenceListing.txt and is 3 KB in size.

### BACKGROUND

Acute graft-versus-host disease (aGVHD) is mediated by alloreactive donor CD4⁺ and CD8⁺T cells after allogenic hematopoietic cell transplantation (HCT)^{1,2}. The gastrointestinal tract is a prominent target of aGVHD, and the severity of damage in the intestine (gut) determines the outcome of gut-aGVHD³. IFN-γ⁺ Th1/Tc1 cells play the dominant role in damaging intestinal Paneth cells that produce Reg3γ, a protein that has bactericidal activity against Gram-positive bacteria⁴ and maintains the homeostasis of intestinal microbiome⁵. aGVHD damage of intestinal epithelial cells and Paneth cells results in dysbiosis that exacerbates gut-aGVHD⁶⁻¹¹.

Corticosteroids are used for initial treatment of aGVHD²⁶. Some patients develop steroid-resistant or refractory (SR) gut-aGVHD, and the pathogenesis of SR-gut-aGVHD remains enigmatic^{27,28}. The prognosis of SR-gut-aGVHD is dismal due to poor understanding of its pathogenesis and lack of effective therapy. Steroid-treatment effectively suppresses Th1/Tc1 but not Th17²⁹, and IL-17A⁺CD4⁺T cells infiltrate the intestinal tissues of patients with SR-gut-aGVHD³⁰. However, targeting steroid-resistant T cells with ATG or anti-CD25 in patients with SR-gut-aGVHD has not been effective²⁷. A recent study with murine models indicated that T cells were dispensable for SR-GVHD pathogenesis³¹. Other studies indicate that while IL-22 from host-type innate lymphocytes augment intestinal epithelial stem cell survival and reduce gut-aGVHD^{32,33}, IL-22 from donor T cells augments gut-aGVHD^{34,35}, although its mechanisms and cellular source (Th17 versus Th22) remains unclear.

Accordingly, there is a need to develop an approach that effectively prevents and treats aGVHD or autoimmune colitis, particularly steroid-resistant aGVHD. This disclosure satisfies the need in the art.

### SUMMARY

This disclosure is directed to a method of preventing or treating aGVHD in a subject receiving a hematopoietic cell transplantation (HCT) or autoimmune colitis. The method entails administering to the subject an effective amount of an anti-IL-22 antibody, an anti-IL-6 antibody, donor-type CX3CR1^{hi} MNPs, donor-type NK cells, a ceacam-1 antagonist, an anti-Gr-1 antibody, or a combination thereof. In certain embodiments, the ceacam-1 antagonist is an anti-ceacam 1 antibody. In certain embodiments, the anti-IL-22 antibody, the anti-IL-6 antibody, the anti-ceacam 1 antibody, or the anti-Gr-1 antibody is a monoclonal antibody. In certain embodiments, the anti-IL-22 antibody, the anti-IL-6 antibody, the anti-ceacam 1 antibody, or the anti-Gr-1 antibody is a recombinant antibody. In certain embodiments, the anti-IL-22 antibody, the anti-IL-6 antibody, the anti-ceacam 1 antibody, or the anti-Gr-1 antibody is a human antibody. In certain embodiments, the anti-IL-22 antibody, the anti-IL-6 antibody, the anti-ceacam 1 antibody, or the anti-Gr-1 antibody is a humanized antibody. In certain embodiments, the aGVHD is a gut aGVHD. In certain embodiments, the aGVHD is a low-risk gut aGVHD that has low serum levels of ST2 and Reg3α. In certain embodiments, the aGVHD is a high-risk gut aGVHD that has high serum levels of ST2 and Reg3α such as a steroid-resistant gut aGVHD. In certain embodiments, the anti-IL-22 antibody, the anti-IL-6 antibody, the donor-type CX3CR1^{hi} MNPs, the donor-type NK cells, the ceacam-1 antagonist, or the anti-Gr-1 antibody is administered to the subject on the same day of receiving HCT. In certain embodiments, the anti-IL-22 antibody, the anti-IL-6 antibody, the donor-type CX3CR1^{hi} MNPs, the donor-type NK cells, the ceacam-1 antagonist, or the anti-Gr-1 antibody is administered to the subject after receiving HCT. In certain embodiments, multiple doses of the anti-IL-22 antibody, the anti-IL-6 antibody, the donor-type CX3CR1^{hi} MNPs, the donor-type NK cells, the ceacam-1 antagonist, or the anti-Gr-1 antibody are administered after HCT. In certain embodiments, a single dose of the anti-IL-22 antibody, the anti-IL-6 antibody, the donor-type CX3CR1^{hi} MNPs, the donor-type NK cells, the ceacam-1 antagonist, or the anti-Gr-1 antibody is administered each day. In certain embodiments, the anti-IL-22 antibody, the anti-IL-6 antibody, the donor-type CX3CR1^{hi} MNPs, the donor-type NK cells, the ceacam-1 antagonist, or the anti-Gr-1 antibody is administered every other day for a week, for two weeks, for three weeks, or for a month after HCT. In certain embodiments, the anti-IL-22 antibody, the anti-IL-6 antibody, the ceacam-1 antagonist, or the anti-Gr-1 antibody is administered to the subject by oral administration or rectal administration. In certain embodiments, the ceacam-1 antagonist such as an anti-ceacam-1 antibody is administered to the subject by oral administration or rectal administration. In certain embodiments, the subject is human.

### BRIEF DESCRIPTION OF THE DRAWINGS

This application contains at least one drawing executed in color. Copies of this application with color drawing(s) will be provided by the Office upon request and payment of the necessary fees.

FIGS. 1a-1f demonstrates establishing a murine model of steroid-resistant acute gut GVHD (SR-gut-aGVHD). Lethally irradiated WT BALB/c recipients were engrafted with splenocytes containing 1.5×10⁶ T cells together with TCD-BM (2.5×10⁶) from WT C57BL/6 donors. Recipients were given a single iv injection of Dexamethasone (DEX, 5 mg/kg) on day 3 (1-DEX) or four total injections on days 3, 10, 15 and 20 (4-DEX) after HCT, with saline used as control. FIG. 1a: Plots of % Original bodyweight and % Survival, n=8 (TCD-BM), 10 (4-DEX), 12 (Saline & 1-DEX) from two replicate experiments. FIG. 1b: Recipient serum concentrations of Reg3γ, ST2 and sTNFRI were measured on day 25 after HCT. Means±SEM are shown, n=4 combined from two replicate experiments. FIGS. 1c-1d: Histopathology of small intestine and colon was evaluated day 25 after HCT. Representative micrographic photos of small intestine (original magnification, ×200), and means±SEM of the numbers of Paneth cell per crypt are showed in FIG. 1c. Representative micrographic photos of colon (original magnification, ×200) and % mice with indicated histopathological scores are shown in FIG. 1d. n=8 per group combined from two replicate experiments. FIGS. 1e-1f: Cytokine profile of T cells from spleen, MLN, and colon were measured on day 7 after HCT. Means±SEM of % IFN-γ⁺ cells among CD4⁺ and CD8⁺T cell subsets are shown in FIG. 1e, and means±SEM of % IL-17A⁺IL-22⁻, IL-17A⁺IL-22⁺ and IL-17A⁻IL-22⁺cells among CD4⁺ and CD8⁺T cell subsets are shown in FIG. 1f. n=4 combined from two experiments. Nonlinear regression (curve fit) was used for body weight comparisons. Log-rank test was used for survival comparisons. One-way ANOVA with Tukey's multiple comparisons test, with the Greenhouse-Geisser correction was used for the comparisons in FIGS. 1b and 1c; Unpaired two-tailed Student's t test corrected for multiple comparisons using the Holm-Sidak method was used to compare means in FIG. 1e. FIG. 1a, ****p<0.0001; FIG. 1b, Reg3γ: *p=0.0133, **p=0.0086, ***p=0.0008; ST2: **p=0.0073(ST2); sTNFRI: *p=0.0208, **p=0.0020(TCD-BM vs 4-DEX), **p=0.0077 (1-DEX vs 4-DEX); FIG. 1c, *p=0.0371 (TCD vs 4-DEX), **p=0.0011 (TCD vs 1-DEX), **p=0.0023 (1-DEX vs 4-DEX); FIG. 1e, IFNγ⁺CD4⁺T: **p=0.0080 (SPL), **p=0.0069 (MLN), ****p<0.0001; IFNγ⁺CD8⁺T: *p=0.0291 (SPL), *p=0.0239 (MLN), ***p=0.00019.

FIGS. 2a-2h show that IL-22 from Th/Tc22 cells were required for induction of SR-gut-aGVHD. FIGS. 2a-2c: Recipients were given 1-DEX, 4-DEX or saline treatment as illustrated in FIG. 1. Cytokine profiles of T cells from spleen and MLN were measured on day 25. Means±SEM of % and yield of IL17A⁺IL22⁻, IL17A⁺IL22⁺, and IL17A⁻IL22⁺ among CD4⁺ and CD8⁺T subsets are shown in FIGS. 2a-2c, respectively. n=4 (1-DEX), 6 (Saline), 7 (4-DEX) combined from two replicates. FIG. 2d: Representative flow cytometry patterns and means±SEM of AHR⁺RORγt⁻ and AHR⁻RORγt⁺ among IL-17A⁺IL-22⁻ or IL-17A⁻IL-22⁺CD4⁺ and CD8⁺ subsets. n=4, two replicates. FIG. 2e: Means±SEM of % and yield of IFNγ⁺CD4⁺ and IFNγ^{+CD}8⁺ T in ileum. n=4, two replicates. FIG. 2f: Recipients engrafted with splenic T from WT or IL-22^{-/-} donors combined with TCD-BM from WT donors were given 4-DEX treatment. The recipients of WT-T were given additional treatment of IL-22 mAb or IgG at 150 μg every 3 days from days 12 to 21 after HCT. On day 25, histopathology of colon was evaluated. Representative micrographs (200×) and % mice with indicated histopathological scores are shown; n=4 (WT-IgG & IL-22^{-/-}T), 12 (WT-anti-IL-22) combined from two replicates. FIG. 2g-2h: Recipients engrafted with T cells from WT or T-RORγt^{-/-} combined with TCD-BM from WT donors were evaluated for cytokine profile of MLN T cells and histopathology of colon on day 25. FIG. 2g: Means±SEM of % and yields of IL17A⁻IL22⁺cells among CD4⁺ and CD8⁺T cell subsets; n=5 (RORγt^{-/-T),} 7 (WT T) combined from two replicates. FIG. 2h: Representative micrographs of colon (200×) and % mice with indicated histopathological scores; n=8, two replicates. P value was determined by Two-way ANOVA with Tukey's (2a-2c) or Sidak's (2d), unpaired two-tailed Student t test (2e and 2g). 2a, ****p<0.0001; yield of IL17A⁺IL22⁻CD4⁺: **p=0.0013, ***p=0.0004; yield of IL17A⁺IL22⁻CD8⁺: *p=0.0416, **p=0.0024 (spleen, saline vs. 4-DEX), **p=0.0074 (MLN, Saline vs 4-DEX); 2c, ****p<0.0001; yield of IL17A⁻IL22⁺CD4⁺: **p=0.0024 (saline vs 4-DEX) **p=0.0050 (1-DEX vs 4-DEX); % IL17A⁻IL22⁺-CD8⁺: *p=0.0256 (saline vs 1-DEX), *p=0.0362 (1-DEX vs 4-DEX); ***p=0.001, ****p<0.0001. Yield of IL17A⁻IL22⁺CD8⁺: ****p<0.0001, **p=0.0040, *p=0.0493. 2d, ****p<0.0001. 2e, *p=0.0155, **p=0.0032. 2g, *p=0.0410, ***p=0.0003, ****p<0.0001.

FIGS. 3a and 3b show that prolonged dexamethasone treatment augmented the expansion of human Th/Tc22 cells in the gut tissues of Xeno-GVHD recipients. RAG2^{-/-}/IL-2Rγ^{-/-} mice were given a single 350 cGy fraction of total body irradiation before injection of 30×10⁶ human PBMC on the same day. On days 3, 6, 9, 12 after HCT, recipients were given a total of 4 injections of dexamethasone (DEX) at 5 mg/kg or saline. On day 15 after HCT, gut tissue of recipients was analyzed for Th/Tc22 cells. FIG. 3a: Flow cytometry gating strategies for human Th/Tc22 cells. FIG. 3b: Means±SEM of percentages and yields of Th/Tc22 are shown. n=4 combined from two replicated experiments. Unpaired two-tailed Student's t test was used to compare means. FIG. 3b, ***p=0.0004 (% Th22), *p=0.0461(Yield of Th22). *p=0.0356 (% Tc22).

FIGS. 4a-4e show that IL-22 from Th/Tc22 cells caused dysbiosis and bacteria translocation. Lethally irradiated WT BALB/c recipients were engrafted with WT TCD-BM alone or combined with splenocytes from WT or IL-22−/−C57BL/6 donors, and the recipients of splenic T cells were given 1-DEX or 4-DEX treatment as illustrated in FIG. 1. Microbiome profile in feces from the ileum of recipients was measured on day 25 after HCT. FIG. 4a: Diversity of ileal flora was determined by the number of species, Shannon index and Fisher Index. Means±SEM are shown. FIG. 4b: Principal coordinate analysis of the ileal flora. FIG. 4c: Bacterial composition at the species level of the ileal flora is depicted with individual mice displayed in each bar. FIG. 4d: % Abundance of E. coli, Enterococcus_sp._FDAARGOS_553 and Lactobacillus murinus in the ileal flora. Means±SEM are shown. FIG. 4e: Bacteria colony numbers in the liver cell suspension culture plates, with one plate per recipient. Means±SEM are shown. All data combined from two replicate experiments, n=9 (TCD-BM), 6 (other groups except TCD-BM). Ordinary one-way ANOVA with Tukey's correction for multiple comparisons was used in 4a and 4e. Kruskal-Wallis test with Dunn's correction for multiple comparisons was used in 4d. 4a, Observed: **p=0.0022 (WT-1-DEX vs. WT-4-DEX), **p=0.0078 (WT-4-DEX vs. IL-22−/−T-4-DEX); Shannon Index: *p=0.0351; Fisher Index: **p=0.0025 (WT-1-DEX vs. WT-4-DEX), **p=0.0077 (WT-4-DEX vs. IL-22−/−T-4-DEX). 4d, **p=0.0016 (1-DEX vs 4-DEX), **p=0.0042 (WT-4-DEX vs. IL-22−/−T-4-DEX); 4e, ****p<0.0001.

FIGS. 5a-5b show that wild-type but not IFNγ^{-/-}CD4⁺ and CD8⁺T cells induced gut-aGVHD with damage to Paneth cells. Lethally irradiated BALB/c recipients were engrafted with TCD-BM (2.5×10⁶) from WT C57BL/6 donors with or without additional whole splenocytes containing 1.5×10⁶ CD4⁺ and CD8⁺T cells from WT or IFN-γ^{-/-}C57BL/6 donors. Recipients were monitored for clinical signs of aGVHD for up to 30 days. FIG. 5a: Curves of % Mice without diarrhea and % Survival are shown. n=8. FIG. 5b: On day 7 after HCT, small intestine tissue was harvested for H&E staining. One representative microphotograph (original magnification 200×) and means±SE of the numbers of Paneth cell per crypt are shown, n=7. All results are combined from 2 replicates. One-way ANOVA was used to compare the means between different groups. Log-rank test was used to compare survival. a, ****p<0.0001; b, **** p<0.0001.

FIGS. 6a-6f show that gut-aGVHD induced by IFNγ^{-/-}donor CD8⁺T cells was associated with expansion of Tc17 and Tc22 cells. Lethally irradiated WT BALB/c recipients were engrafted with splenocytes containing 1.5×10⁶ T cells combined with TCD-BM (2.5×10⁶) from WT or IFNγ^{-/-}C57BL/6 donors. Additional controls include IFNγ^{-/-}C57BL/6 grafts in syngeneic WT C57BL/6 recipients and MHC I-matched H-2K^{b}MHC-IA⁻-IE⁻BALB/c recipients. Allogeneic recipients were also treated with depleting anti-CD4 mAb (500 μg/mouse) immediately after HCT (FIGS. 6a-6c). FIG. 6a: Plots show % Mice without diarrhea, % Original bodyweight, and % Survival. n=8 (syngeneic & MHC I-matched recipients), 10 (IFNγ^{-/-}CD8⁺ and WT CD8⁺). FIG. 6b: Seven and 14 days after HCT, histopathology of colon was evaluated. Representative micrographic photos and % Mice with indicated colitis histopathology scores are shown; n=4 per group. FIG. 6c: At day 7 after HCT, MLN of recipients were analyzed for donor-type IL-17A⁺IL-22⁻, IL-17A⁺IL-22⁺ or IL-17A⁻IL-22⁺CD8⁺T cell subsets. Means±SE of percentages are shown, n=10. FIGS. 6d-6f: Lethally irradiated WT BALB/c recipients were engrafted with sorted CD8⁺T (1.5×10⁶) from IFNγ^{-/-} or IFNγ^{-/-}/RORγt^{-/-}C57BL/6 donors combined with TCD-BM (2.5×10⁶) from WT C57BL/6 donors. FIG. 6d: Plots of % Mice without diarrhea and % Survival are shown. n=8. FIGS. 6e-6f: On day 7 after HCT, MLN cells were analyzed for % IL17A⁻IL22⁺CD8⁺T cells (6e) and % AHR⁺RORγt⁻ or AHR⁻RORγt⁺ among IL17A⁻IL22⁺CD8⁺T cells (6f). Means±SEM are shown, n=4. All results combined from two replicates. Log-rank test was used for survival comparisons. Nonlinear regression (curve fit) was used for body weight and diarrhea comparisons. Unpaired two-tailed Student t test was used to compare means. 6a, ****p<0.0001; 6c, **p=0.0063, ***p=0.0006; 6d, ****p<0.0001; 6e, **p=0.0010 (IFNγ^{-/-}), **p=0.0059 (IFNγ^{-/-}/RORγt^{-/-}).

FIGS. 7a-7c show that IFNγ^{-/-} donor CD8⁺T cells induced gut-aGVHD without damaging Paneth cells. Lethally irradiated BALB/c recipients were engrafted with TCD-BM with or without splenocytes from WT or IFNγ^{-/-}C57BL/6 donors, as illustrated in FIG. 6a. FIG. 7a: Representative microphotographic photos (original magnification 200×) of ileum H&E staining at 7 and 14 days after HCT, with arrows pointing to Paneth cells, n=4. FIG. 7b: At day 7 after HCT, Paneth cells in the crypts were counted with enlarged photos on the computer screen. Means±SE of Paneth cell numbers/Crypt of recipients given TCD-BM alone, WT CD8⁺T, or IFNγ^{-/-}CD8⁺T are shown, n=4-8, combined from two replicate experiments. FIG. 7c: At day 7 after HCT, ileal tissue from recipients given WT CD8⁺T cells or IFN-γ^{-/-}CD8⁺T cells were tested for mRNA expression of Defensins (Defcr1 and Defcr3). Means±SE of 5 recipients/group are shown. Unpaired two-tailed Student's t tests were used to compare means. 7c, *p<0.0377.

FIGS. 8a-8k show that gut-aGVHD induced by IFN-γ^{-/-} donor CD8⁺T cells was Tc22-dependent. FIGS. 8a-8b: gut-aGVHD was induced in BALB/c recipients with grafts from IFN-γ^{-/-} or IFN-γ^{-/-}/IL-17^{-/-}C57BL/6 donors as illustrated in FIG. 6a. FIG. 8a: Plots of % Mice without diarrhea, % Original bodyweight and % Survival. n=8. FIG. 8b: Means±SEM of percentages of IL-17A⁻IL-22⁺CD8⁺T cells in MLN at 7 days after HCT are shown. n=4. FIGS. 8c-8e: WT BALB/c recipients with gut-aGVHD induced with grafts from IFNγ^{-/-}C57BL/6 donors were treated with anti-IL-22 mAb or control IgG. FIG. 8c: Plots of % Mice without diarrhea and % Survival; n=10. FIG. 8d: Mean±SEM of % IL-17A⁺IL-22⁻, IL-17A⁺IL-22⁺ or IL-17A⁻IL-22⁺CD8⁺T cell subsets in MLN at day 7. n=8. FIG. 8e: % and yield of neutrophils in the colon at day 10. n=6 (IgG), 7 (anti-IL-22). FIGS. 8f and 8g: WT BALB/c with grafts from WT or IFNγ^{-/-}C57BL/6 donors were measured for ileal expression of Reg3γ and serum Reg3γ at days 7 and 14. Mean±SEM, n=4 (8f, d7), 6 (8f, d14, WT), 9 (8f, d14, IFNγ^{-/-} and 8g, d14, WT), 8 (8g, d7, WT), 11 (8g, d7, IFNγ^{-/-}), 7 (8g, d14, IFNγ^{-/-}). FIG. 8h: Mean±SEM of Ileal Reg3γmRNA and serum Reg3γ in anti-IL-22 and IgG groups at day 7. n=5 (mRNA), 10 (Serum, IgG), 7 (Serum, anti-IL-22). FIGS. 8i-8k: Lethally irradiated WT or Reg3γ^{-/-}C57BL/6 recipients were engrafted with WT TCD-BM (2.5×10⁶) and CD8⁺T cells (2.5×10⁶) from IFN-γ^{-/-}BALB/c donors, and recipients for monitored for gut-GVHD and survival. FIG. 8i: Day 7, Mean±SEM of % IL-17A⁺IL-22⁻, IL-17A⁺IL-22⁺ or IL-17A⁻IL-22⁺CD8⁺T cells in MLN, n=9. FIG. 8j: Day 7, means±SEM of ileal Reg3γmRNA expression is shown, n=5. FIG. 8k: Plots of % Survival, n=8 (WT), 6 (Reg3γ^{-/-}). Results are combined from two replicates. Nonlinear regression (curve fit) was used for body weight and diarrhea comparisons. Log-rank test was used for survival comparison. Unpaired two-tailed Student t test was used to compare means between two groups. 8a, ****p<0.0001; 8c, ***p=0.003, ****p<0.0001; 8e, **p=0.0022, ***p=0.0010; 8f, ****p<0.0001, *p=0.0127; 8g, *p=0.0103, **p=0.0063; 8h, **p=0.0047, ****p<0.0001; 8j. **p=0.0031; 8k. **p=0.0021.

FIG. 9 shows neutralization of IL-22 prevented gut-aGVHD induced by IFN-γ^{-/-} donor CD8⁺T cells. Lethally irradiated WT BALB/c recipients were engrafted with splenocytes containing T cells (1.5×10⁶) from IFN-γ^{-/-}C57BL/6 donors together with TCD-BM (2.5×10⁶) from WT C57BL/6 donors and treated with anti-CD4 mAb (500 μg/mouse) on the day of HCT. Recipients were also treated with anti-IL-22 mAb or control mouse IgG (150 μg/mouse), every other day from days 0 to 6 after HCT. At day 14 after HCT, colon sections were stained with H&E. The boxed areas are shown for colonic epithelial cell damage and lamina propria infiltration in recipients treated with control mouse IgG but not in recipients treated with anti-IL-22. Micrograph photos are representative from 1 of 4 replicate experiments.

FIGS. 10a-10b show that IL-22 from alloreactive T cells induced lethal aGVHD. Lethally irradiated BALB/c recipients were engrafted with splenocytes containing T cell (1.5×10⁶) from WT or IL-22^{-/-}C57BL/6 donors together with TCD-BM cells (2.5×10⁶) from WT C57BL/6 donors. Recipients were treated with anti-CD4 Ab (1 mg/mouse) on day 0 and daily anti-IFNγ (1 mg/mouse) from days 0 to 5 after HCT. Recipients were monitored for clinical signs of aGVHD for up to 30 days. FIG. 10a: Plots of % Mice without diarrhea and % Survival are shown; n=10. FIG. 10b: At days 6 after HCT, liver suspensions were cultured for bacterial colony formation. Means±SE of bacterial colonies/culture are shown, n=5-6 per group. Unpaired two-tailed Student t tests were used to compare means. Log-Rank test was used to compare survival. 10a, ****p<0.0001; 10b, **p=0.0037.

FIGS. 11a-11b show that IL-22-producing host-type ILC3 cells were eliminated before gut-aGVHD onset. Lethally irradiated BALB/c recipients were given splenocytes from IFNγ^{-/-}C57BL/6 donors together with TCD-BM from WT C57BL/6 donors as illustrated in FIG. 6a. Yields of ILC3 cells in the small intestine (11a) and colon (11b) on day 1, 3, 5, 7 and 10 days after HCT are shown. Mean±SE, n=4. All results combined from two replicate experiments. One-way ANOVA was used to compare means between multiple groups. 11a, ****p<0.0001; 11b, **p=0.0016.

FIGS. 12a-12d show that TBI-conditioning was required for induction of gut-aGVHD by Tc22 cells in a haploidentical HCT model. CB6F1 recipients with or without TBI-conditioning (1300cGy) were engrafted with CD4⁺T-depleted splenocytes containing 2×10⁶ CD8⁺T cells from IFNγ^{-/-}C57BL/6 donors together with 2.5×10⁶ TCD-BM from WT C57BL/6 donors. The recipients given TBI-conditioning were treated with IL-22 mAb or control IgG (150 μg/mouse) on days 0, 2, 4 and 6 after HCT. Recipients were monitored for clinical signs of aGVHD for up to 30 days. FIG. 12a: Plots of % Original bodyweight change, % Mice without diarrhea, and % Survival are shown, n=8-9. FIGS. 12b and 12c: On day 30 after HCT, small intestine and colon sections stained with H&E were analyzed by histopathology. One representative of micrograph (original magnification, 200×) of small intestine (12b) and colon (12c) and means±SEM of GVHD pathological scores are shown; n=8. FIG. 12d: Mesenteric lymph node (MLN) from TBI-conditioned or non-conditioned recipients were harvested on day 30 after HCT. Means±SE of percentages and yield of IL17A⁺IL22⁻, IL17A⁺IL22⁺ and IL17A⁻IL22⁺CD8⁺T cell subsets are shown. n=4. All results are combined from two replicates. Log-rank test was used for comparison of survival. Unpaired two-tailed Student t tests were used to compare means between groups. 12a, *p<0.0133; 12d, *p=0.0129, **p=0.0028, ****p<0.0001.

FIGS. 13a-13c show that Reg3γdeficiency in recipients ameliorated SR-gut-aGVHD. Lethally irradiated WT or Reg3γ^{-/-}C57BL/6 recipients were given splenocytes contained 1.5×10⁶ T cells and 2.5×10⁶ TCD-BM cells from WT BALB/c donors. Recipients were treated with DEX (5 mg/kg) on days 3, 10, 15 and 20 after HCT and were monitored for clinical signs of GVHD for up to 50 days. FIG. 13a: Plots of % Original bodyweight and % Survival are shown. n=9. FIG. 13b: On day 50 after HCT, the length of colon was measured, and means±SE are shown. n=9. FIG. 13c: At day 50 after HCT, H&E stained colon sections were analyzed by histopathology. One representative microphotograph (original magnification 200×) and the distributions of histopathology scores are shown. All results are combined from 2 replicate experiments. Unpaired two-tailed Student t tests were used to compare means. Nonlinear regression was used to compare body weights between 2 groups. 13a, **p=0.0045; 13b, **p=0.0012.

FIGS. 14a-14e show that Tc22 cells caused dysbiosis via IL-22 and Reg3γ. FIGS. 14a-14c: Gut-aGVHD was induced in BALB/c recipients with grafts from WT or IFNγ^{-/-}C57BL/6 donors as illustrated in FIG. 6a. Recipients of IFNγ^{-/-} grafts were treated with anti-IL-22 mAb or control IgG (150 μg/mouse) every other day from day 0 to day 6 after HCT. Feces from the ileum of untreated BALB/c, recipients of WT CD8⁺T, and recipients of IFNγ^{-/-}CD8⁺T treated with anti-IL-22 or control IgG were analyzed for microbiome profiles with 16S RNA-seq on day 6. FIG. 14a: % Abundance of Clostridiaceae, Streptococcus, Lactobacillus and Escherichia/Shigella. Means±SEM are shown, n=9 (untreated BALB/c, 3 replicates), 20 (WT CD8⁺T, 4 replicates), 24 (IFNγ^{-/-}CD8⁺T+IgG, 5 replicates), 10 (IFNγ^{-/-}CD8⁺T+anti-IL-22, 2 replicates). FIGS. 14b and 14c: Liver suspensions were cultured on 3 blood agar plates for 24 hours. Means±SEM of enumerated bacteria colonies from each liver are shown in FIG. 14b. n=20 (WT CD8⁺T & IFNγ^{-/-}CD8⁺T+anti-IL-22, 4 replicates), 45 (IFNγ^{-/-}CD8⁺T+IgG, 9 replicates). Genus-level bacterial composition of colonies grown from liver suspension of IFNγ^{-/-}CD8⁺T recipients are displayed in each bar are shown in FIG. 14c, n=9. FIGS. 14d-14e: Lethally irradiated WT or Reg3γ^{-/-}C57BL/6 recipients were engrafted with IFNγ^{-/-}BALB/c CD8⁺T cells (2.5×10⁶) combined with TCD-BM (2.5×10⁶) from WT BALB/c donors. Six days after HCT, ileal feces were analyzed with bacteria 16S RNA sequencing, and liver cell suspensions were cultured. FIG. 14d: % Abundance of Barnesiella, Escherichia/Shigella and Blautia. Means±SEM are shown. n=11 (Reg3γ^{-/-}), 12 (WT). 2 replicate experiments. FIG. 14e: Mean±SE of bacteria colony numbers for each liver. n=11. 2 replicates. P value was determined by Kruskal-Wallis with Dunn's multiple comparisons test (14a), ordinary one-way ANOVA (14b), Mann-Whitney test (14d) and unpaired two-tailed Student's t test (14e). 14a, *p=0.0362, ****p<0.0001; 14b, *p=0.0354 (WT CD8⁺T vs. IFNγ^{-/-}CD8⁺T+IgG), *p=0.0443 (IFNγ^{-/-}CD8⁺T+IgG vs. IFNγ^{-/-}CD8⁺T+anti-IL-22); 14d, ***p=0.0003 (Escherichia/Shigella); **p=0.0070; ***p=0.0004 (Blautia); and 14e, **p=0.0053.

FIG. 15 shows comparison of intestinal microbiota. A supplement to FIG. 14a. Representative bacterial composition of ileal flora at the genus level.

FIGS. 16a-16c show that dysbiosis was required for gut-aGVHD induced by IFNγ^{-/-}CD8⁺T-derived Tc22 cells. Gut-aGVHD was induced in BALB/c recipients with grafts from WT or IFNγ^{-/-}C57BL/6 donors as illustrated in FIG. 6a. FIGS. 16a-16b: Starting on the day of HCT, recipients of IFNγ^{-/-}CD8⁺T cells were separately housed or co-housed with recipients of WT CD8⁺T cells. FIG. 16a: Plots of % mice without diarrhea, % Original bodyweight and % Survival, n=12 combined from two replicate experiments. FIG. 16b: % Abundance of Escherichia/Shigella, Clostridiaceae and Lactobacillus from ileal fecal samples. n=6 (co-house IFNγ^{-/-}CD8⁺T), 8 (non-co-house IFNγ^{-/-}CD8⁺T and WT CD8⁺T) from two replicate experiments. FIG. 16c: Recipients of IFNγ^{-/-}CD8⁺T cells were gavaged with a mixture of ampicillin(1 g/L), neomycin(1 g/L), metronidazole(1 g/L), and vancomycin (0.5 g/L) (4ABX) or PBS (250 μl/mouse/day) from days 0-7 following HCT. Plots of % Mice without diarrhea, % Original bodyweight and % survival are shown. n=10 from two replicate experiments. Pvalue was determined by nonlinear regression (curve fit) (16a and 16c, body weight and diarrhea), log-rank test (16a and 16c, survival), Kruskal-Wallis test with Dunn's correction for multiple comparisons (16b). 16a, ****p<0.0001; 16b, *p=0.0463, ***p=0.0002; and 16c, *p=0.0480, ***p=0.0002.

FIGS. 17a and 17b show that donor-derived CX3CR1^{hi} cells had a mononuclear phagocyte phenotype. Lethally irradiated BALB/c recipients received HCT with splenocytes from WT or IFNγ^{-/-}C57BL/6 donors as illustrated in FIG. 18a. 10 days after HCT, gated CX3CR1^{hi} and CX3CR1^{lo} H-2^{b+}CD11c⁺CD11b⁺cells from colon tissues were analyzed for expression of F4/80, CD64, MerTK, IL-10R and CSF1-R. FIG. 17a: Flow cytometry gating strategy. FIG. 17b: One representative histogram is shown of 5 per group, combined from two replicate experiments.

FIGS. 18a-18e show that gut-aGVHD induced by IFNγ^{-/-}CD8⁺T cells was associated with depletion of donor-type CX3CR1^{hi} MNP via PD-1. Gut-aGVHD was induced in BALB/c recipients with grafts from WT or IFNγ^{-/-}C57BL/6 donors as illustrated in FIG. 6a. FIG. 18a: Ten days after HCT, colonic lamina propria mononuclear cells were analyzed for percentage and yield of donor-type CD11c⁺CD11b⁺CX3CR1^{hi} MNP. Representative patterns and means±SEM of percentage and yield are shown. n=8. FIG. 18b: CX3CR1^{hi} and CX3CR1^{lo} MNP from recipients given WT and IFNγ^{-/-}CD8⁺T cells are shown in histogram of PD-1 staining; one representative pattern is shown, n=5. FIG. 18c: Apoptosis of CX3CR1^{hi} MNP from the recipients given WT or IFNγ^{-/-}CD8⁺T cells was measured by Annexin V staining. A representative staining pattern and means±SEM of percentage of Annexin V⁺cells are shown. n=5. FIG. 18d: Recipients given IFNγ^{-/-}CD8⁺T cells were treated with anti-PD-L1 mAb or control IgG (400 μg/mouse) on days 0, 3 and 6 after HCT. On day 10, the percentages of CX3CR1^{hi} MNP and percentages of Annexin V⁺CX3CR1^{hi} MNP were measured. One representative staining pattern and means±SEM are shown. n=4 (% Annexin V⁺), 10 (% CX3C1R1^{hi}). FIG. 18e: Recipients given IFNγ^{-/-}CD8⁺T were treated with anti-IL-22 mAb or control IgG (150 μg/mouse) every other day from days 0-6 after HCT. On day 10, the yield of CX3CR1^{hi} MNP and their expression of PD-1 were measured. Means±SEM of percentage of CX3CR1^{hi} MNP and MFI are shown. n=10. All results are combined from two replicate experiments; unpaired two-tailed Student t test was used to compare means between two groups. 18a, **p=0.0039, ****p<0.0001; 18c, **p=0.0033; and 18d, **p=0.0045, ***p=0.0007.

FIGS. 19a-19b show that colon epithelial cells expressed low levels of PD-L1 in recipients given IFNγ^{-/-}-splenocytes. Lethally irradiated WT BALB/c recipients were engrafted with splenocytes (5×10⁶) and TCD-BM (2.5×10⁶) from IFNγ^{-/-}C57BL/6 donors and were treated with anti-CD4 mAb (500 μg/mouse) on day 0 after HCT. On day 10 after HCT, PD-L1 expressed on colon epithelial cells (CK⁺CD45⁻) was measured. FIG. 19a: Gating strategies for PD-L1 expression on colon epithelial cells. FIG. 19b: Mean±SE of MFI of PD-L1 is shown. n=5 per group, combined from two replicate experiments. Unpaired two-tailed Student's t test was used to compare means. (****p<0.0001).

FIGS. 20a-20h show that gut-aGVHD induced by IFNγ^{-/-}CD8⁺T cells was reversed by preservation of donor-type CX3CR1^{hi} MNP. FIGS. 20a-20c: WT or PD-L1^{-/-}BALB/c recipients were engrafted with IFNγ^{-/-}splenocytes and WT TCD marrow cells from C57BL/6 donors as illustrated in FIG. 6a. FIG. 20a: Plots of % Mice without diarrhea, % Original bodyweight and % Survival are shown. n=8 (PD-L1^{-/-} recipients with IFNγ^{-/-}CD8⁺T), 9 (WT recipients with WT or IFNγ^{-/-}CD8⁺T). 2 replicate experiments. FIG. 20b: Percentage and yield of CX3CR1^{hi} MNP in colon tissues on day 10 after HCT. One representative staining pattern and means±SEM are shown, n=5. 2 replicate experiments. FIG. 20c: Means±SEM of bacteria colony numbers in liver suspension cultures from WT or PD-L1^{-/-} recipients 7 days after HCT. n=15. 3 replicate experiments. FIGS. 20d-20g: PD-L1^{-/-}BALB/c recipients were transplanted with sorted IFNγ^{-/-}CD8⁺T cells with WT or CX3CR1^{-/-}TCD-BM from C57BL/6 donors. 2 replicate experiments. FIG. 20d: Plots of % Mice without diarrhea and % survival are shown. n=9. FIG. 20e: Representative flow cytometry pattern and % CX3CR1^{hi} MNP in colon tissue. Means±SEM are shown, n=4. FIG. 20f: Mean±SEM of % IL17A⁻IL22⁺CD8⁺T cells in MLN on day 7, n=6. FIG. 20g: Bacteria colony numbers in MLN and liver cell suspension from recipients of WT or CX3CR1^{-/-}TCD-BM cells. Means±SEM of CFU are shown, n=8. FIG. 20h: Splenic CX3CR1^{hi} or CX3CR1^{lo} MNP cells (0.5×10⁶) from PD-L1^{-/-}BALB/c recipients of IFNγ^{-/-}CD8⁺T on day 10 after HCT were sorted and then iv. injected into WT BALB/c recipients of IFNγ^{-/-}CD8⁺T cells on day 1 after HCT. The WT Recipients were monitored for clinical signs of aGVHD for up to 30 days, and plots of % Original bodyweight, % Mice without diarrhea and % Survival are shown. n=6 from two replicate experiments. P value was determined by non-linear regression (20a, 20d, and 20h, body weight & diarrhea), log-rank test (20a, 20d, and 20h, survival), nonparametric Mann-Whitney test (20c and 20g), unpaired two-tailed Student t test (20b, 20e, 20f). 20a, ****p<0.0001; 20b, *p=0.0271 (%), *p=0.0329 (yield); 20c, *p=0.0104; 20d, **p=0.0038, ****p<0.0001; 20e, *p=0.0336; 20f, **p=0.0044; 20g, *p=0.0286, ***p=0.0002; and 20h, **p=0.0043, ****p<0.0001.

FIGS. 21a-21c show that SR-gut-GVHD was associated with depletion of CX3CR1^{hi} MNP. FIG. 21a: WT BALB/c recipients were engrafted with spleen and BM cells from WT C57BL/6 donors and were treated with 1-DEX or 4-DEX, as illustrated in FIG. 1. Means±SEM of the percentage and yield of donor-derived (H-2Kb⁺) CX3CR1^{hi} MNP cells in the colon of DEX-1 or DEX-4 recipients at day 25 after HCT are shown. n=6 (1-DEX), 12 (4-DEX). FIGS. 21b and 21c: Lethally irradiated WT BALB/c recipients were given TCD-BM (2.5×10⁶) from CX3CR1^{+/-}or CX3CR1^{-/-}C57BL/6 donors together with splenocytes (5×10⁶) from CX3CR1^{+/-}C57BL/6 donors. Recipients were given a single i.v. injection of dexamethasone (5 mg/kg) on day 3. FIG. 21b: % Survival, n=9 (CX3CR1^{+/-}BM), 10 (CX3CR1^{-/-}BM). FIG. 21c: At day 7 after HCT. Means±SEM of % IL-17A⁻IL-22⁺cells among CD4⁺and CD8⁺T cell from MLN are shown. n=4. All results combined from two replicated experiments. Unpaired two-tailed Student t test was used to compare means between groups. Log-rank test was used for survival comparisons. 21a, **p=0.018 (%), **p=0.011 (yield); 21b, **p=0.0093; and 21c, **p=0.0035 (CD4⁺T), **p=0.0018 (CD8⁺T).

FIG. 22 is a diagram depicting the pathogenesis of (SR-gut-aGVHD). Dexamethasone (DEX) treatment reduces IFNγ⁺Th/Tc1 differentiation and preferentially expands the numbers of IL-17⁻IL-22⁺Th/Tc22, particularly Tc22 cells. Reduction of Th/Tc1 ameliorates damage in small intestine, and expansion of Th/Tc22 cells augments damage in the lower intestinal track such as colon. The IL-22 from Th/Tc22 cells causes dysbiosis in a Reg3γ-dependent manner and augments neutrophil infiltration in the colon tissue. DEX treatment also reduces gut tissue CX3CR1^{hi} MNP that is important for controlling bacteria translocation. Dysbiosis, enhanced neutrophil infiltration, loss of CX3CR1^{hi} MNP and bacterial translocation cause full-blown SR-gut-aGVHD in the colon.

FIGS. 23a-23g show flow cytometry gating strategies. FIG. 23a: Gating strategies for mouse Th1/Tc1 cells used in FIGS. 1e and 2e. FIG. 23b: Gating strategies for mouse Th/Tc22 and Th/Tc17 cells used in FIG. 1f. FIG. 23c: Gating strategies for mouse Th/Tc22 and Th/Tc17 cells used in FIGS. 2a-2d, 2g, 6c, 6e, 6f, 8d, and 12d. FIG. 23d: Gating strategies for mouse neutrophils used in FIG. 8e. FIG. 23e: Gating strategies for mouse ILC3 used in FIG. 11. FIG. 23f: Gating strategies for mouse CX3CR1^{hi} MNP used in FIGS. 17, 18, and 20. FIG. 23g: Gating strategies for mouse CX3CR1^{hi} MNP used in FIG. 21.

FIGS. 24A-24D shows that severe IL-22-producing T cells infiltrated the colon tissue of SR-Gut-GVHD patient. FIG. 24A: Representative micrograph of H&E-stained colon biopsy tissues of patients with mild gut GVHD, moderate gut GVHD, or severe SR-gut-GVHD. Scale bars, 100 μm. FIG. 24B: Representative mass cytometry image of severe SR-gut-GVHD patient colon tissue showing expression of the indicated stromal markers, immune markers, Ki-67, Granzyme B and DNA by the cells. FIG. 24C: Representative mass cytometry images of severe SR-gut-GVHD patient colon tissue showing expression of DAPI (blue), CD3 (cyan), CD4 (green), CD8α (green) and IL-22 (red), and the overlay of CD3, CD4, IL-22 and DAPI or CD3, CD8α, IL-22 and DAPI. FIG. 24D: Representative mass cytometry images of severe SR-gut-GVHD patient colon tissue showing expression of DAPI (blue), CD3 (white), CD4 (red), CD8α (green), IL-22 (cyan), IFN-γ (magenta), and IL-17A (yellow). Scale bar (200 μm) in panels 24B-24D.

FIGS. 25A-25D show that the colon tissues of patients with mild or moderate gut-GVHD were with few IL-22+T cell infiltration. FIGS. 25A-25B: Representative mass cytometry image of mild gut-GVHD (25A) and moderate gut-GVHD (25B) in the colon tissues, showing expression of the indicated stromal markers, immune markers, Ki-67, Granzyme B and DNA by the cells. FIGS. 25C-25D: Representative mass cytometry images of mild gut-GVHD (25C) and moderate gut GVHD (25D) colon tissues, showing expression of DAPI (blue), CD3 (white), CD4 (red), CD8α (green), IL-22 (cyan), IFN-γ (magenta), and IL-17A (yellow). Scale bar (200 μm).

FIG. 26 shows that ceacam-1 expression was upregulated on the colon epithelia cells of the mice with SR-Gut-GVHD. Lethally irradiated WT BALB/c recipients were given T cell depleted bone marrow cells (TCD-BM, 2.5×10⁶) with or without splenocytes (2.5×10⁶) from WT C57BL/6 donors. Recipients engrafted with TCD-BM and splenocytes together were given a single i.v. injection of Dexamethasone (DEX, 5 mg/kg) on day 3 (1-DEX) or four total injections on days 3, 10, 15 and 20 (4-DEX) after HCT. Immunohistochemistry (INC) staining of ceacam-1(brown) on colon tissues of mice with or without SR-Gut-GVHD was evaluated on day 25 after HCT. One representative micrographic photo of colon tissue (original magnification, ×100) is shown of 4 replicate experiment in each group.

FIGS. 27A-27G show that host ceacam-1 deficiency ameliorated SR-Gut-GVHD but not non-SR-GVHD. FIG. 27A: Lethally irradiated WT or ceacam-1^{-/-}BALB/c recipients were engrafted with splenocytes (2.5×10⁶) together with TCD-BM (2.5×10⁶) from WT C57BL/6 donors. Plots of % of original bodyweight, % of mice without diarrhea, and % of survival are shown. n=15 from two replicate experiments. FIG. 27B: Lethally irradiated BALB/c WT recipients (WT-Rec) or ceacam-1^{-/-}recipients (ceacam-1^{-/-}-Rec) were engrafted with cells from WT C57BL/6 donors as shown in panel 27A, recipients were given total four injections of DEX on days 3, 10, 15 and 20 (4-DEX) after HCT, plots of % of original bodyweight, % of mice without diarrhea, and % of survival are shown. n=10 from two replicate experiments. FIG. 27C: Histopathology of colon was evaluated day 25 after HCT. Representative micrographic photos of colon (original magnification, ×200) and % of mice with indicated histopathological scores are shown. n=4 per group combined from two replicate experiments. FIG. 27D: Means±SEM of yield of CD11b⁺ly6G⁺cells among total mononuclear cells are shown. FIG. 27E: Means±SEM of yield of H2Kb⁺TCRβ⁺CD4⁺ and CD8⁺T cell are shown. n=4 combined from two experiments. FIG. 27F: Means±SEM of % of ceacam-1⁺H2Kb⁻cells among CD45⁻cell are shown. FIG. 27G: Immunohistochemistry (INC) staining of ceacam-1 (purple), CD11b (yellow) and CD3 (teal) in the colon tissue of TCD-BM, WT and ceacam-1^{-/-}recipients was evaluated day 25 after HCT. Representative micrographic photos of colon (original magnification, ×100) are shown. Nonlinear regression (curve fit) was used for body weight comparisons. Log-rank test was used for survival comparisons. Unpaired two-tailed Student's t test was used to compare means in 27D, 27E and 27F.

FIGS. 28A-28H show that ceacam-1 deficiency on host intestinal epithelia cell but not host hematopoietic cells ameliorated SR-Gut-GVHD. FIGS. 28A-28C: Lethally irradiated WT BALB/c recipients were engrafted with TCD-BM (10×10⁶) from WT or ceacam-1^{-/-}BALB/c donors to generate the WT-Chimeras or host hematopoietic cell (HC)-ceacam-1^{-/-}-chimeras. Two months after bone marrow reconstitution, WT-Chimeras or HC-ceacam-1^{-/-}-Chimeras were engrafted with splenocytes (2.5×10⁶) together with TCD-BM (2.5×10⁶) from WT C57BL/6 donors. FIG. 28A: Plots of % of original body weight and % of mice without diarrhea, n=18 (WT chimeras), 15 (host HC-ceacam-1^{-/-}-Chimeras from two replicate experiments. FIG. 28B: Histopathology of colon was evaluated day 25 after HCT. Representative micrographic photos of colon (original magnification, ×200) and % of mice with indicated histopathological scores are shown. n=6 for WT chimeras and n=8 for Host HC-ceacam-1^{-/-}-Chimeras, combined from two replicate experiments. FIG. 28C: Immunohistochemistry (INC) staining of ceacam-1 (purple), CD11b (yellow) and CD3 (teal) on colon of WT-Chimeras and host HC-ceacam-1^{-/-}-Chimeras was evaluated day 25 after HCT. Representative micrographic photos of colon (original magnification, ×100) are shown. Means±SEM of % of ceacam-1⁺area among the whole slide is shown. FIGS. 28D-28H: Lethally irradiated WT and ceacam-1^{-/-}BALB/c recipients were engrafted with TCD-BM (10×10⁶) from WT BALB/c donors to generate the WT-Chimeras or IEC-ceacam-1^{-/-}-Chimeras. Two months after bone marrow reconstitution, WT-Chimeras or IEC-ceacam-1^{-/-}-Chimeras were engrafted with splenocytes (2.5×10⁶) together with TCD-BM (2.5×10⁶) from WT C57BL/6 donors. FIG. 28D: Plots of % of original bodyweight and % of mice without diarrhea, n=18 (WT chimera), 15 (Host IEC ceacam-1^{-/-}chimera) from two replicate experiments. FIG. 28E: Immunohistochemistry (INC) staining of ceacam-1 (purple), CD11b (yellow) and CD3 (teal) on colon of WT chimera and host IEC ceacam-1^{-/-}chimera recipients was evaluated day 25 after HCT. Representative micrographic photos of colon (original magnification, ×100) are shown. Means±SEM of % ceacam-1⁺area among the whole slide is shown. FIG. 28F: Histopathology of colon was evaluated day 25 after HCT. Representative micrographic photos of colon (original magnification, ×200) and % of mice with indicated histopathological scores are shown. n=4 (WT chimera), 5 (Host IEC ceacam-1^{-/-}chimera) combined from two replicate experiments. FIGS. 28G and 28H: Means±SEM of % and yield of CD4⁺ and CD8⁺T cells in mesenteric lymph node (28G) and colon intraepithelial (28H) are shown. Unpaired two-tailed Student's t test was used to compare means in 28E, 28F and 28H.

FIGS. 29A-29H show that ceacam-1 deficiency in host intestinal epithelial cells led to trans-differentiation of pathogenic Th/Tc22 cells into iTregs and inhibition of Th/Tc1 cell expansion in MLN. WT-Chimeras or IEC ceacam-1^{-/-}-Chimeras were engrafted with splenocytes together with TCD-BM from WT C57BL/6 donors and induced to develop SR-Gut-GVHD, as shown in FIG. 28. FIG. 29A: Means±SEM of % and yield of IL-22⁺IL17A⁻CD4⁺(Th22) and IL-22⁺IL17A⁻CD8⁺T (Tc22) cells in mesenteric lymph node (MLN). n=22 (% WT), 18(% IEC-ceacam-1^{-/-}), 18 (Yield of WT), 14 (Yield of IEC-ceacam-1^{-/-}). FIG. 29B: Umap plot of 8 clusters generated from IL-22⁺IL17A⁻CD4⁺in MLN of both WT- and IEC ceacam-1^{-/-}Chimeras. Means±SEM of % individual populations among total are shown. FIG. 29C: Means±SEM of % of population 4 (Pop 4) and population 5 (Pop 5) are shown. FIG. 29D: Heatmap plot of IFN-γ, CD127, T-bet, GM-CSF, AHR, IL-2, CCR6, FoxP3, PD-1, IL-17A, RORγt, ceacam-1 and IL-10 expression on Th22 cells in individual populations are shown. FIG. 29E: Representative flow cytometry pattern and gating strategy of FoxP3, AHR, RORγt and IL-10 expression in the Th22 cells. FIG. 29F: Means±SEM of % and yield of FoxP3⁺AHR⁻, FoxP3⁺RORγt⁻, FoxP3⁻RORγt⁺ and IL10⁺Foxp3^{hi} Th22 subsets. FIG. 29G: Representative flow cytometry pattern and gating strategy of PD-1, CCR6, IL-2 and T-bet expression comparison in the FoxP3⁺RORγt⁻and Foxp3⁻RORγt⁺ Th22 subsets. FIG. 29H: Representative flow cytometry pattern and Means±SEM of % and yield of T-bet⁺IFN-γ⁺CD4⁺T cells are shown. n=10 (WT-chimeras), 8 (IEC-ceacam-1^{-/-}-chimeras) combined from two replicate experiments. Unpaired two-tailed Student's t test was used to compare means in 29C, 29F, 29G and 29H.

FIGS. 30A-30G show that ceacam-1 deficiency in the host intestinal epithelial cells reduced Th/Tc22 and Th/Tc1 cells while increasing Tregs in the colon tissues. WT-Chimeras or IEC-ceacam-1^{-/-}-Chimeras were engrafted with splenocytes together with TCD-BM from WT C57BL/6 donors as described in FIG. 28. FIG. 30A: Means±SEM of % and yield of IL-22⁺IL17A⁻CD4⁺(Th22) and IL-22⁺IL17A⁻CD8⁺T (Tc22) cells among colon intraepithelial cells. FIG. 30B: Representative flow cytometry pattern and Means±SEM of % and yield of RORγt⁺IL-10⁻and RORγt⁻IL-10⁺CD4⁺T cells among colon intraepithelial cells are shown. FIG. 30C: Representative flow cytometry patterns and gating strategy of IL-17A, PD-1 and T-bet expression in the RORγt⁺IL-10⁻and RORγt⁻IL-10⁺CD4⁺T cells among colon intraepithelial cells. FIG. 30D: Representative flow cytometry patterns and Means±SEM of % and yield of T-bet⁺IFN-γ⁺CD4⁺and CD8⁺T cells among colon intraepithelial cells are shown. FIG. 30E: Representative flow cytometry patterns and Means±SEM of % and of FoxP3⁺IL-22⁻and FoxP3⁻IL-22⁺CD4⁺T cells among colon laminar propria cells. FIG. 30F: Gated from FoxP3⁺IL-22⁻CD4⁺T cells, representative flow cytometry pattern of IL-10⁺CCR6⁻, IL-10⁺RORγt⁺, T-bet+IL-10+and IL-17A-IL-10+cells are shown. FIG. 30G: Representative flow cytometry patterns and Means±SEM of % and yield of T-bet⁺IFN-γ⁺CD4⁺and CD8⁺T cells among colon laminar propria are shown. n=8 (WT-Chimeras), 6 (IEC-ceacam-1^{-/-}Chimeras) combined from two replicate experiments (30A, 30B and 30D). n=4 combined from two replicate experiments (30E and 30G). Unpaired two-tailed Student's t test was used to compare two means.

FIGS. 31A-31D show that ceacam-1 deficiency in host intestinal epithelial cells reversed dysbiosis and inhibited E. coli and ceacam-1⁺IEC interaction. WT- or IEC-ceacam-1^{-/-}-Chimeras were engrafted with splenocytes together with TCD-BM from WT C57BL/6 donors as described in FIG. 28. FIG. 31A: Diversity of ileal flora was determined by the number of species, Chao1 index, ACE index, Shannon index, Simpson index, Invsimpson index and Fisher Index. Means±SEM are shown. FIG. 31B: Principal coordinate analysis of the ileal flora. FIG. 31C: Bacterial composition at the species level of the ileal flora is depicted with individual group displayed in each bar. FIG. 31D: Immunofluorescence staining (IF) ceacam-1 (green) and E. coli LPS (red) in the colon. One representative micrographic photo of colon (Scale bar 20 μm) is shown of 4 replicate experiments.

FIGS. 32A-32G show that combination of in vivo anti-ceacam-1 and anti-Gr-1 administration ameliorated SR-GI-GVHD. Lethally irradiated WT BALB/c recipients were engrafted with splenocytes (2.5×10⁶) together with TCD-BM (2.5×10⁶) from WT C57BL/6 donors, recipients were given total four injections of dexamethasone on days 3, 10, 15 and 20 (4-DEX) after HCT, recipients were also given i.p. injection anti-Gr-1 (0.5 mg/mouse, day 11) with or without anti-ceacam-1 (anti-CC1) (0.1 mg/mouse, days 12, 15, 17, 20) or control IgG. FIG. 32A: Plots of of original bodyweight, % of mice without diarrhea and % of survival, n=21 (IgG), 9 (anti-Gr-1), 19 (anti-CC1) and 8 (anti-Gr-1+anti-CC1) from two replicate experiments. FIG. 32B: Means±SEM of colon length is show. n=2-4 from two replicate experiments. FIG. 32C: Histopathology of colon was evaluated day 25 after HCT. Representative micrographic photos of colon (original magnification, ×100 and ×200) and % of mice with indicated histopathological scores are shown. n=4 per group combined from two replicate experiments. FIG. 32D: Representative flow cytometry pattern and Means±SEM of % and yield of FoxP3⁺RORγt⁻and FoxP3⁻RORγt⁺CD4⁺T cells. FIG. 32E: Representative flow cytometry pattern and gating strategy of FoxP3, RORγt and IL-10 expression in the Th22 cells. Means±SEM of % and yield of FoxP3⁺RORγt⁻and IL10⁺Foxp3^{hi} Th22 subsets. n=4-12 combined from two replicated experiments (32C and 32D). FIG. 32F: Representative flow cytometry pattern and gating strategy of PD-1, CCR6, IL-2 and T-bet expression comparison in the FoxP3⁺RORγt⁻and Foxp3⁻RORγt⁺Th22 subsets. Means±SEM of MFI are shown. n=20 combined from 3 experiments. FIG. 32G: Immunofluorescence staining (IF) ceacam-1 (green) and E. coli LPS (red) in the colon. Representative micrographic photos of colon (original magnification, ×400) are shown. Nonlinear regression (curve fit) was used for body weight comparisons. Log-rank test was used for survival comparisons. One-way ANOVA was used to compare means in 32B, 32C and 32E. Unpaired two-tailed Student's t test was used to compare means in 32F.

FIG. 33 shows colon infiltrated neutrophils expressing extremely high level of ceacam-1 compared to Th1 cells. Lethally irradiated WT BALB/c recipients were given T cell depleted bone marrow cells (TCD-BM) (2.5×10⁶) with splenocytes (2.5×10⁶) from WT C57BL/6 donors. Recipients were given 4-DEX administration as shown in FIG. 26. Flow cytometry analysis of ceacam-1 expression on donor Th1 (T-bet⁺IFN-γ⁺) cells and neutrophils in the colon on day 25 after HSCT. Means±SEM of MFI are shown. n=4 combined from duplicated experiments. Unpaired two-tailed Student's t test was used to compare means.

### DETAILED DESCRIPTION

The studies by the inventor(s) show that prolonged steroid treatment leads to reduction of IFN-γ-producing Th1/Tc1 cells and preferential expansion of Th/Tc22 cells, resulting in dysbiosis. On the other hand, lack of IFN-γ also leads to depletion of protective donor-type CX3CR1^{hi} MNP. The concurrence of dysbiosis and loss of protective CX3CR1^{hi} MNP result in full-blown SR-gut-GVHD with excessive infiltration of neutrophils and other inflammatory cells in the colon tissue. Neutralizing IL-22 or transfer of donor-type CX3CR1^{hi} MNP can prevent the disease. IL-6 can augment Th22 differentiation. Donor IFN-γ-producing NK cells can prevent GVHD while preserving GVL activity. Therefore, neutralizing IL-6 or blockade of IL-6R signaling and infusion of NK cells can prevent induction of SR-gut-GVHD; and neutralizing IL-22 or blockade of IL-22R signaling and infusion of donor-type CX3CR1^{hi} MNP can reverse or ameliorate SR-gut-GVHD. The methods disclosed herein can also prevent or treat autoimmune colitis.

Accordingly, this disclosure is directed to a method of preventing or treating acute GVHD (aGVHD) in a subject receiving a hematopoietic cell transplantation (HCT) or autoimmune colitis . The method entails administering to the subject an effective amount of an anti-IL-22 antibody, an anti-IL-6 antibody, or donor-type CX3CR1^{hi} mononuclear phagocytes (MNPs) or NK cells harvested from the donor's peripheral blood. In certain embodiments, the anti-IL-22 antibody or the anti-IL-6 antibody is a monoclonal antibody. In certain embodiments, the anti-IL-22 antibody or the anti-IL-6 antibody is a recombinant antibody. In certain embodiments, the anti-IL-22 antibody or the anti-IL-6 antibody is a human antibody. In certain embodiments, the anti-IL-22 antibody or the anti-IL-6 antibody is a humanized antibody. In certain embodiments, the aGVHD is a gut aGVHD. In certain embodiments, the aGVHD is a low-risk gut aGVHD that has low serum levels of ST2 and Reg3a. In certain embodiments, the aGVHD is a high-risk gut aGVHD that has high serum levels of ST2 and Reg3αsuch as a steroid-resistant gut aGVHD. The MAGIC algorithm probability of evaluating serum levels of ST2 and Reg3αas biomarkers for aGVHD was previously disclosed.⁶¹ In certain embodiments, low risk aGVHD has a biomarker median and range of Reg3α: 24 (8-458) or ST2: 16972 (2977-74233). In certain embodiments, high risk aGVHD has a biomarker median and range of Reg3α: 98 (8-1141) or ST2: 64709 (24069-199143).

“Treating” or “treatment” of a disease or a condition may refer to preventing the disease or condition, slowing the onset or rate of development of the disease or condition, reducing the risk of developing the disease or condition, preventing or delaying the development of symptoms associated with the disease or condition, reducing or ending symptoms associated with the disease or condition, generating a complete or partial regression of the disease or condition, or some combinations thereof.

As used herein, the term “subject” refers to a mammalian subject, preferably a human. The phrases “subject” and “patient” are used interchangeably herein.

An “effective amount,” “therapeutically effective amount” or “effective dose” is an amount of a composition (e.g., an antibody or a pharmaceutical composition) that produces a desired therapeutic effect in a subject, such as preventing or treating a target disease or condition, or alleviating symptoms associated with the disease or condition. The precise therapeutically effective amount is an amount of the composition that will yield the most effective results in terms of efficacy of treatment in a given subject. This amount will vary depending upon a variety of factors, including but not limited to the characteristics of the active agent (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of an active agent and adjusting the dosage accordingly. For additional guidance, see Remington: The Science and Practice of Pharmacy 21st Edition, Univ. of Sciences in Philadelphia (USIP), Lippincott Williams & Wilkins, Philadelphia, PA, 2005.

The administration schedule and doses of the anti-IL-22 or anti-IL-6 antibody can be determined based on the need of the subject. For example, the anti-IL-22 or anti-IL-6 antibody is administered to the subject immediately before or on the same day of receiving HCT. In certain embodiments, the anti-IL-22 or anti-IL-6 antibody is administered to the subject after receiving HCT. In certain embodiments, the anti-IL-22 or anti-IL-6 antibody is administered to the subject receiving HCT at the onset of GVHD. In certain embodiments, multiple doses of the anti-IL-22 or anti-IL-6 antibody are administered after HCT. In certain embodiments, a single dose of the anti-IL-22 or anti-IL-6 antibody is administered each day. In certain embodiments, the anti-IL-22 or anti-IL-6 antibody is administered every other day for a week, for two weeks, for three weeks, or for a month after HCT. One of ordinary skill in the art would understand that when multiple doses of the anti-IL-22 or anti-IL-6 antibody is administered, each dosage may be the same or different. For example, a higher dosage may be administered immediately after HCT and followed by a lower dosage at a later time, e.g., after a week of administration on every other day. Alternatively, a lower dosage may be administered first, followed by a higher dosage. In some embodiments, the donor-type CX3CR1^{hi} MNPs and/or NK cells can be administered to the subject after receiving HCT. In some embodiments, the donor-type CX3CR1^{hi} MNPs and/or NK cells can be administered to the subject on the same day of receiving HCT. In certain embodiments, one or more antibodies can be administered before, on the same day, or after administration of donor-type CX3CR1^{hi} MNPs and/or NK cells. Various combinations of antibodies (anti-IL-22 antibody and/or anti-IL-6 antibody) and/or donor-types cells (donor-type CX3CR1^{hi} MNPs and/or NK cells) disclosed herein can be used in treating aGVHD or autoimmune colitis, in particular, gut aGVHD, steroid-resistant aGVHD, or SR gut aGVHD. In certain embodiments, the anti-IL-22 antibody is administered to high-risk aGVHD patients. In certain embodiments, the anti-IL-6 antibody is administered to low-risk or high-risk aGVHD patients.

Any suitable administration route of the antibody may be chosen. For example, the anti-IL-22 or anti-IL-6 antibody can be administered to the subject by intravenous, intradermal, subcutaneous, intramuscular, intraperitoneal, intranodal, or intrasplenic administration. In certain embodiments, donor-type CX3CR1^{hi} MNPs and/or NK cells are administered to the subject by infusion.

Whether or not donor T cells mediate SR-gut-aGVHD and how IFN-γ^{-/-}donor CD8⁺T cells mediate lethal aGVHD are decades-old questions without answers. Using SR-gut-aGVHD model and murine gut-aGVHD models induced by IFN-γ^{-/-}CD8⁺T cells disclosed herein, two mechanisms have been discovered: dysbiosis mediated by expansion of Th/Tc22 cells and depletion of CX3CR1^{hi} MNP mediated by PD-1. This document discloses new insights into the pathogenesis of SR-gut-aGVHD thereby providing a therapy and/or prophylactic for aGVHD, in particular, SR-gut-aGVHD.

With murine models of SR-gut-aGVHD, it is shown in this disclosure that SR-gut-aGVHD pathogenesis is associated with reduction of IFN-γ⁺Th/Tc1 cells and preferential expansion of IL-17⁻IL-22⁺Th/Tc22, particularly Tc22 cells. The IL-22 from Th/Tc22 cells causes dysbiosis in a Reg3γ-dependent manner. Furthermore, IFN-γ deficiency in donor CD8⁺T cells alone allows for preferential expansion of alloreactive Tc22 and subsequent dysbiosis. The IFN-γ deficiency also leads to depletion of intestinal protective CX3CR1^{hi} mononuclear phagocytes (MNP) in a PD-1-dependent manner, and depletion of CX3CR1^{hi} MNP augments expansion of Tc22 cells. Simultaneous dysbiosis and depletion of CX3CR1^{hi} MNP results in full-blown gut-aGVHD.

Under inflammatory conditions, enhanced production of IL-22 and Reg3γ in gut tissues can also induce dysbiosis and pathogen colonization¹². IL-22 in gut tissues can be produced by innate lymphocytes, NK and NKT cells, as well as Th17 and Th22 cells^{13,14}. Th/Tc17 cells include IL-17A⁺IL-22⁻and IL-17A⁺IL-22⁺subsets, and their differentiation is regulated by RORα and RORc (RORγt)^{15,16}. Th/Tc22 differentiation is regulated by AHR, and in Th22 cells, AHR expression is augmented by RORγt and suppressed by T-bet¹⁷. Th/Tc22 cells are IL-22⁺IL-17A⁻¹⁷, and human Th22 cells can be IL-22^{hi}IFN-γ^{lo}IL-17A⁻¹⁸.

In healthy individuals, CX3CR1⁺intestinal mononuclear phagocytes (MNP) play an important role in clearing entero-invasive pathogens and preventing pathogen translocation from the intestinal lumen into mesenteric lymph nodes and the liver¹⁹⁻²¹. CX3CR1⁺MNP also promote epithelial barrier repair²² and regulate Th17 and Treg differentiation^{23,24}. Recipient-derived hematopoietic cells including CX3CR1⁺MNP are targets of aGVHD and are replaced with donor-derived cells²⁵.

Disclosed herein are the roles of Th/Tc17 and Th/Tc22 as well as CX3CR1^{hi} MNP in the pathogenesis of SR-gut-aGVHD. The studies indicate that dysbiosis mediated by Th/Tc22 expansion and depletion of CX3CR1^{hi} MNP plays a critical role in the pathogenesis of SR-gut-aGVHD.

First, IFN-γ deficiency in donor T cells and prolonged steroid treatment preferentially expand alloreactive Th/Tc22 in the gut tissue of GVHD recipients. It was previously reported that alloreactive T cells reciprocally differentiated into Th1, Th2, and Th17, and Th1 and Th17 preferentially infiltrated the gut tissues^{52,53}. It was also reported that IL-17A⁺IL-22⁺Tc17 cells were a transient lineage occurred only early after HCT⁵⁴, although AHR⁺Tc22 cells has not yet been reported. As demonstrated in the working examples, the donor IFN-γ^{-/-}CD8⁺T cells preferentially differentiated into RORγt⁺IL-17A⁺IL-22⁻Tc17 and AHR⁺IL-17A⁻IL-22⁺Tc22 cells in the TBI-conditioned but not non-conditioned recipients. The expansion of Tc22 may result from increased release of IL-6 and IL-1β from TBI-damaged tissues⁴⁶ that augmented Th/Tc22 differentiation and from reduction of T-bet expression in IFN-γ^{-/-}T cells that inhibit Th/Tc22 differentiation¹⁷. Similarly, prolonged steroid treatment also preferentially augmented Th/Tc22 expansion but reduced Th/Tc17 expansion. This may be due to that steroid treatment reduced tissue release of TGF-β but not IL-6³⁷. Reduction of TGF-β could inhibit Th/Tc17 differentiation or expansion but augment Th/Tc22 expansion^{17,40}. Reduction of IFN-γ⁺Th1/Tc1 could also augment expansion of Th/Tc22 by reducing T-bet-mediated inhibition¹⁷. Interestingly, steroid-resistant colitis is associated with high serum IL-6 concentrations³⁷. Thus, it is of interest to investigate whether blockade of IL-6R signaling reduces Th/Tc22 expansion and prevent induction of SR-Gut-aGVHD.

Second, expansion of Th/Tc22 causes dysbiosis and gut-aGVHD. Host cell derived IL-22 augmented intestinal epithelial stem cell and Paneth cell survival and expansion^{8,32} and control intestinal microbiome homeostasis via augmenting Reg3γsecretion^{47,55}. Gut-aGVHD caused by Th1 and Tc1 cells resulted in reduction of Reg3γ, dysbiosis and exacerbation of gut-aGVHD^{6,7,10}. In contrast, over-production of IL-22 and Reg3γunder inflammatory situation was also found to augment pathogen colonization in the gut tissues¹². As demonstrated in the working examples, gut-aGVHD caused by expansion of Th/Tc22 cells was associated with little or reduced damage in small intestinal epithelium and Paneth cells in the recipients of IFN-γ^{-/-}CD8⁺T cells or recipients with SR-gut-aGVHD. Instead, the gut-aGVHD caused by Th/Tc22 cells was associated with severe infiltration of neutrophils in the colon tissues, and the pathogenesis required IL-22- and Reg3γ-dependent dysbiosis. These results show that either under or over production of IL-22 and Reg3γcan induce dysbiosis that contributes to the pathogenesis of gut-aGVHD. Interestingly, although positive response with administration of IL-22 was 100% in low-risk gut-aGVHD patients, it was less than 50% in high-risk patients⁵⁶. Patients with SR-gut-aGVHD belongs to high-risky. Consistent with pathogenic role of neutrophils^{43,44}, the anti-IL-22 treatment markedly reduced neutrophil infiltration in the colon tissues of gut-aGVHD induced by Tc22 cells. Therefore, there is a need for caution in clinical testing of IL-22 as a therapy for high-risk aGVHD, e.g., gut-aGVHD or colitis, especially those patients with potential SR-gut-aGVHD or colitis.

Third, IFN-γ deficiency in donor T cells as well as reduction of IFN-γ⁺Th/Tc1 cells by steroid treatment led to depletion of CX3CR1^{hi} MNP in the gut tissue of GVHD recipients; in addition, lack of donor T-derived IFN-γ led to CX3CR1^{hi} MNP upregulating expression of PD-1 and becoming more sensitive to tissue PD-L1-induced apoptosis. These results indicate that donor T-derived IFN-γ is a double-edged sword that can either augment gut-aGVHD by direct damage to Paneth and intestinal stem cells⁵⁷ or reduce gut-aGVHD by augmenting expansion of protective CX3CR1^{hi} MNP. There is a need for caution in testing combinations of agents that concurrently inhibit IFN-γR and JAK2 signaling⁵⁸.

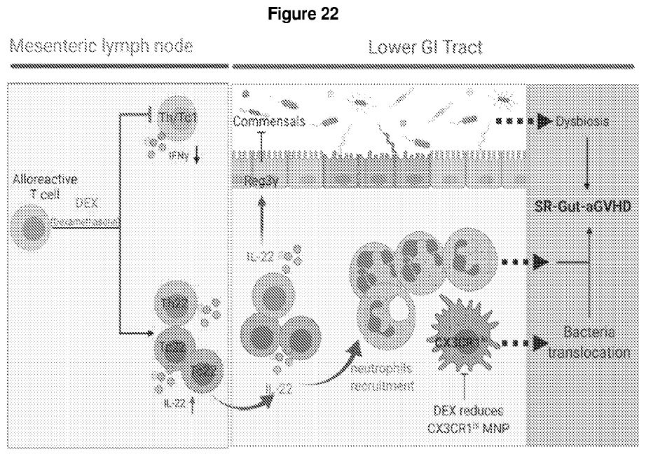

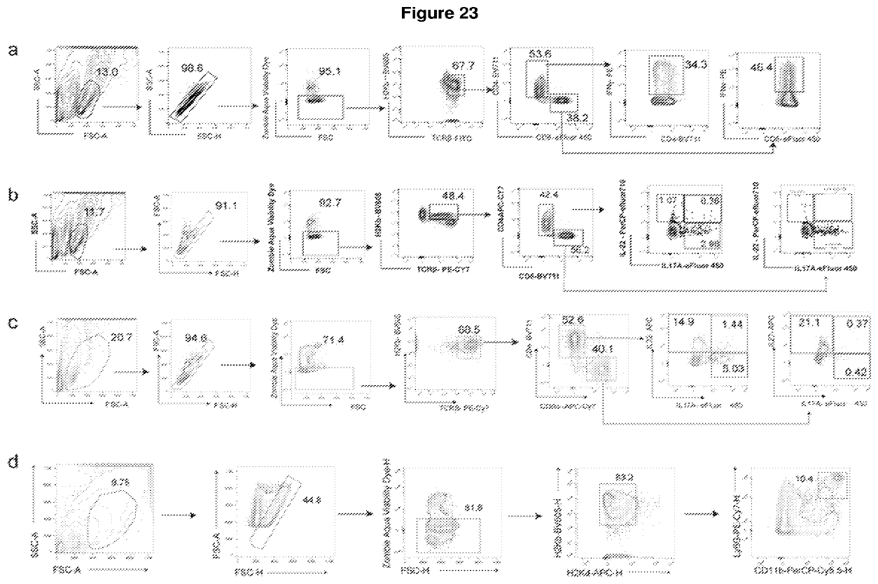

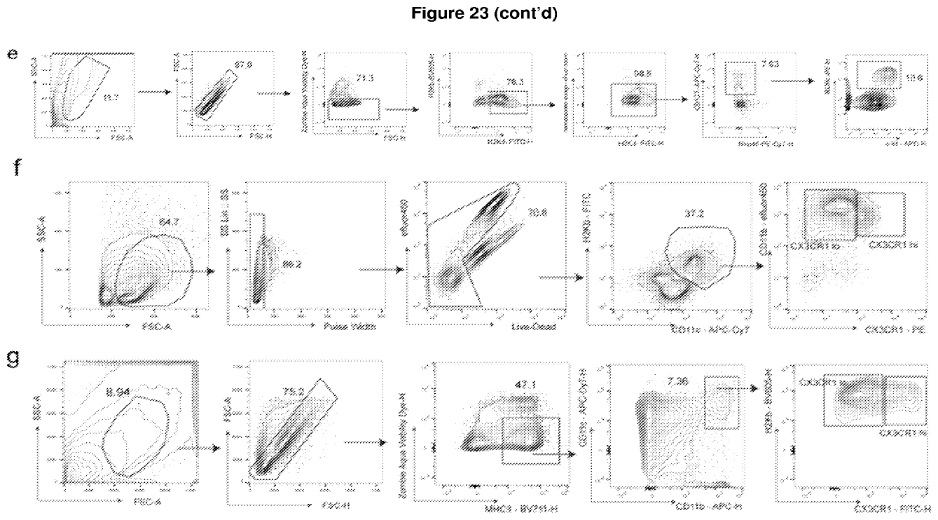

Finally, either elimination of intestinal bacterial or preservation/transfer of CX3CR1^{hi} MNP prevented induction of gut-aGVHD induced by expansion of Th/Tc22 cells. Therefore, as depicted in the diagram (FIG. 22), simultaneous dysbiosis triggered by expansion of Th/Tc22 and reduction of protective CX3CR1^{hi} MNP associated with lack of donor T-derived IFN-γ can result in SR-gut-aGVHD. These studies open a new avenue towards understanding pathogenesis and developing novel approaches for preventing and treating SR-gut-aGVHD.

From the foregoing, it will be appreciated that specific embodiments of the invention have been described herein for purposes of illustration, but that various modifications may be made without deviating from the scope of the invention. The examples are set forth to aid in understanding the invention but are not intended to, and should not be construed to, limit its scope in any way. The examples do not include detailed descriptions of conventional methods. Such methods are well known to those of ordinary skill in the art and are described in numerous publications. All references mentioned herein are incorporated in their entirety.

### Example 1. Materials and Methods

Mice: BALB/c (H-2^{d}) and C57BL/6 (H-2^{b}) mice were purchased from the National Cancer Institute (Frederick, MD). PD-L1^{-/-}BALB/c (H-2^{d}) breeders were provided by Dr. Lieping Chen (Yale University). Reg3γ^{-/-}C57BL/6 (H-2^{b}) breeders were provided by Dr. James Ferrara (Mount Sinai Hospital, NY). IFN-γ^{-/-}C57BL/6 (H-2^{b}), IFN-γ^{-/-}BALB/c (H-2^{d}), CB6F1 (H-2^{b/d}), IL-22^{-/-}C57BL/6 (H-2^{b}), B6(Cg)-Rorctm3Litt/J, B6.Cg-Tg(Cd4-cre)1Cwi/BfluJ, Rag2⁻γc⁻and CX3CR1^{-/-}C57BL/6 were purchased from the Jackson Laboratory (Bar Harbor, ME). CX3CR1^{-/-}C57BL/6 mice were mated with WT C57BL/6 mice to generate CX3CR1^{+/-}mice. B6(Cg)-Rorctm3Litt/J mice were mated with B6.Cg-Tg(Cd4-cre)1Cwi/BfluJ mice to generate T-RORγt^{-/-}C57BL/6 (H-2^{b}) mice. RORγt^{-/-}C57BL/6 (H-2^{b}) mice were provided by Dr. Zuoming Sun (City of hope, Duarte). IFN-γ^{-/-}C57BL/6 mice were mated with RORγt^{-/-}C57BL/6 mice to generate the IFN-γ^{-/-}/RORγT^{-/-}C57BL/6 (H-2^{b}) mice. IFN-γ^{-/-}IL-17^{-/-}C57BL/6 (H-2^{b}) mice were established as previously described⁵². H-2Kb⁺IA⁻IE⁻BALB/c mice were generated by backcrossing MHCII^{-/-}C57BL/6⁵⁹ mice into WT BALB/c mice for more than 12 generations. All mice were maintained in a pathogen-free room in the City of Hope Animal Research Center. All animal protocols were approved by the City of Hope Institutional Animal Care and Use Committee (IACUC).

Murine GVHD model: In general, mice were used at 8 to 12 weeks of age, BALB/c recipients were exposed to 850 cGy total body irradiation in a single fraction, and C57BL/6 and CB6F1 recipients were exposed to 1300 cGy total body irradiation in a single fraction. Splenocytes and T cell depleted bone marrow cells from donors were injected via tail vein into recipients 6-8 hours after irradiation. Dexamethasone (5 mg/kg) was given by i.v. injection on day 3 alone or on days 3, 10, 15 and 20 after HCT. Depletion of T cells from the bone marrow was accomplished by using biotin-conjugated anti-CD4 and anti-CD8 mAbs, and streptavidin Microbeads (Miltenyi Biotec, Germany), followed by passage through an autoMACS Pro cell sorter (Miltenyi Biotec, Germany). Microbeads (Ly-2, Miltenyi Biotec, Germany) were used for to purify CD8⁺T cell, and purity was>99%. The assessment and scoring of clinical signs of GVHD have been described in previous publications⁶⁰.

Xeno-GVHD model: Rag2⁻γc⁻mice were used at 8 to 12 weeks of age. Mice were given a single intravenous injection of clodronate liposomes at 0.1 ml/mouse one day before irradiation and were exposed to 350 cGy total body irradiation in a single fraction before injection of human PBMCs on the same day. Human PBMCs were isolated by Ficoll Paque Plus (GE healthcare) density centrifugation and washed twice in phosphate-buffered saline (PBS). Cells were then counted and suspended in PBS at 30×10⁶ cells/0.2 ml. Cell suspensions containing 30×10⁶ cells were injected via the tail vein.

Isolation of lamina propria cells from mouse intestine: Longitudinal sections the small intestine or colon were processed using a gentleMACS Dissociator and mouse lamina propria dissociation kit (both Miltenyi Biotec), according to the manufacturer's protocol.

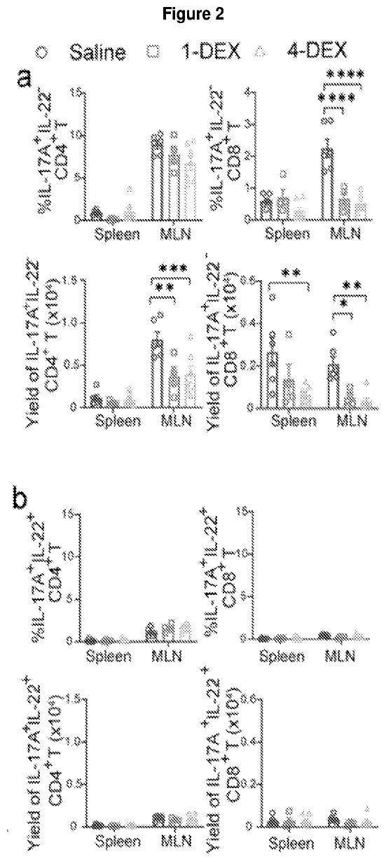

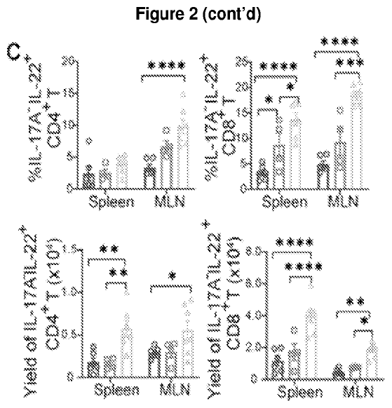

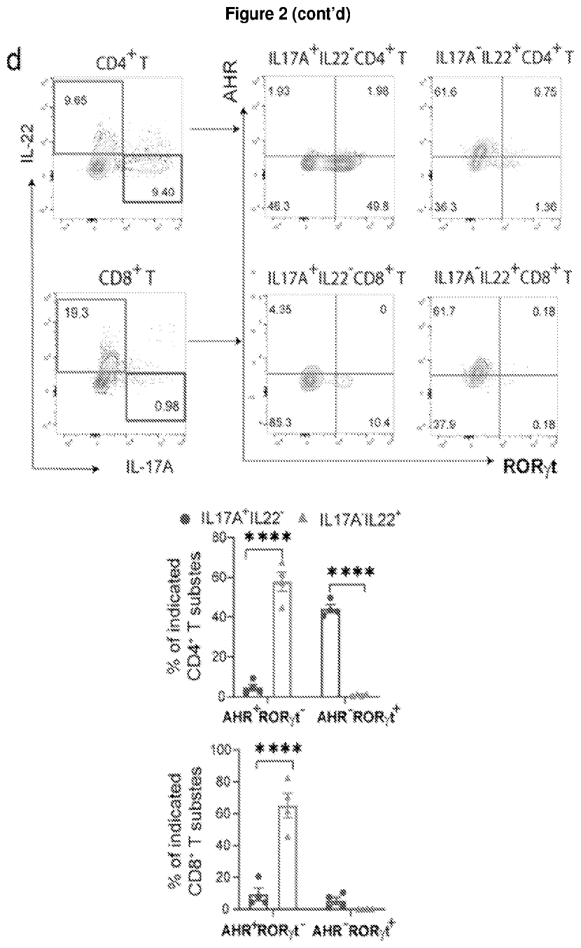

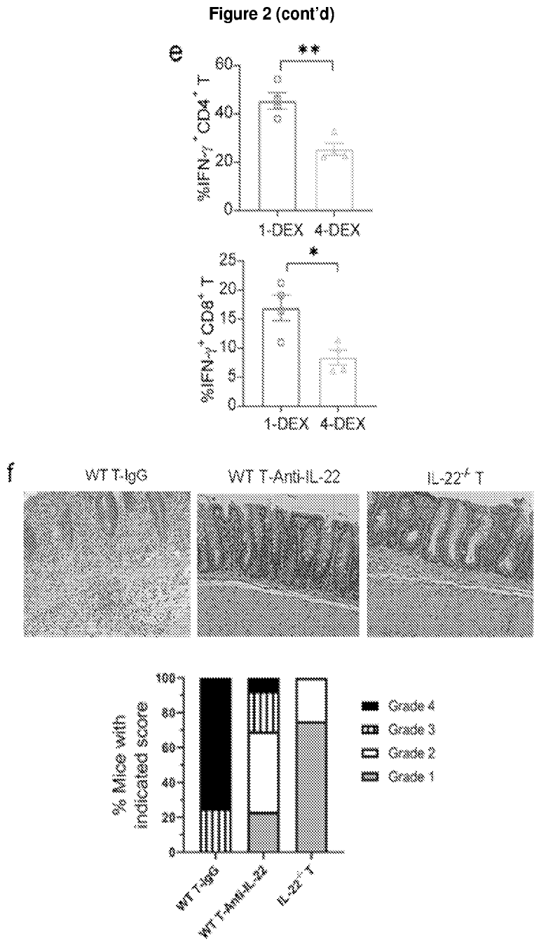

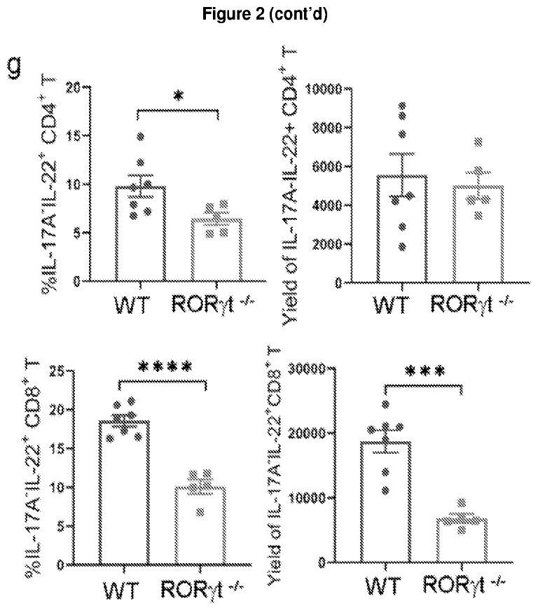

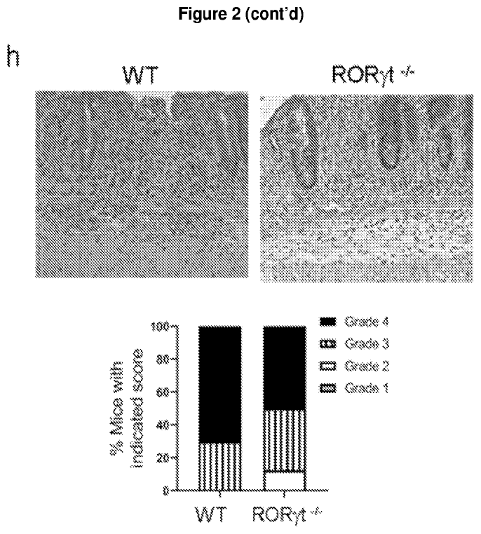

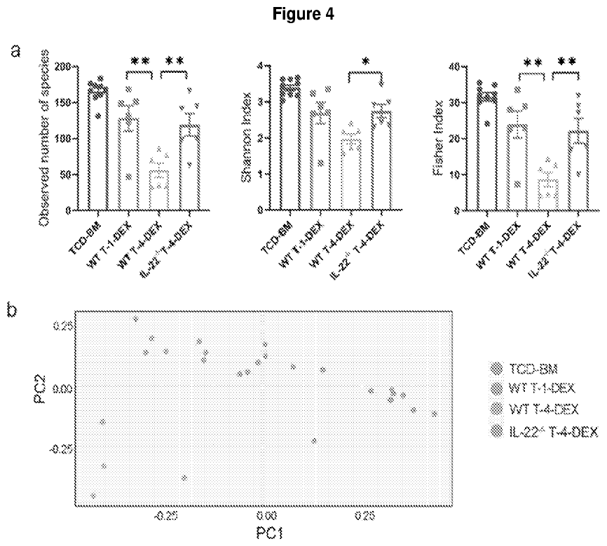

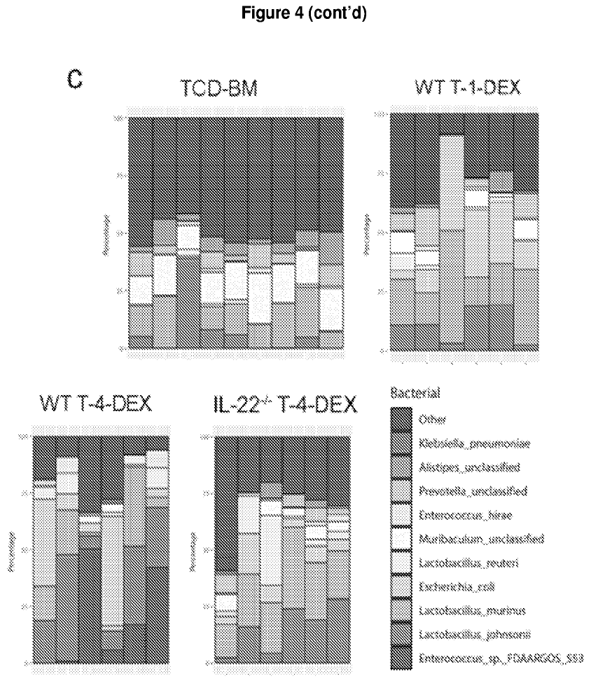

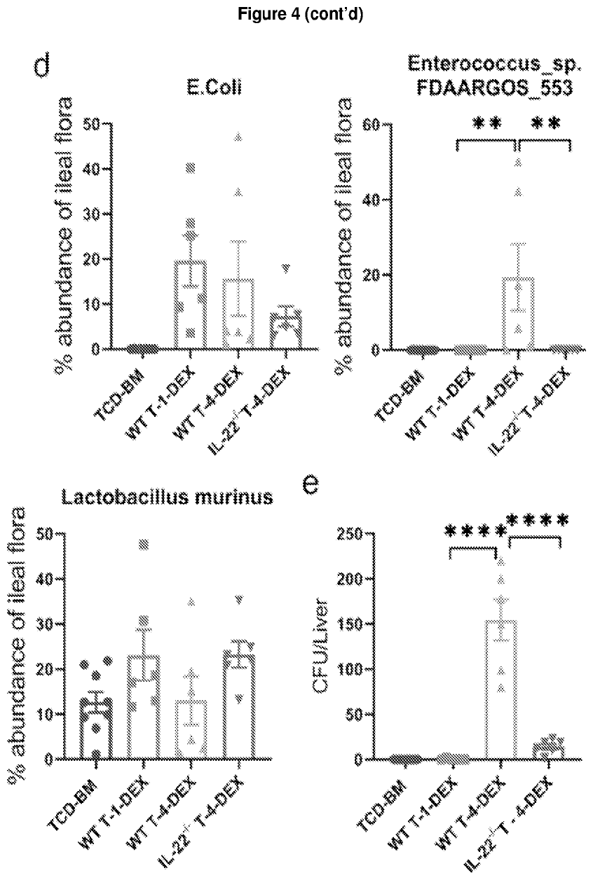

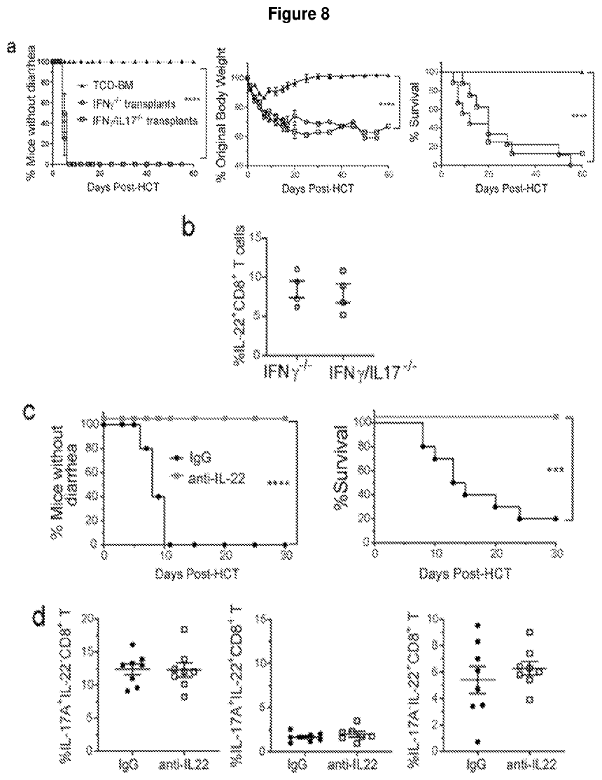

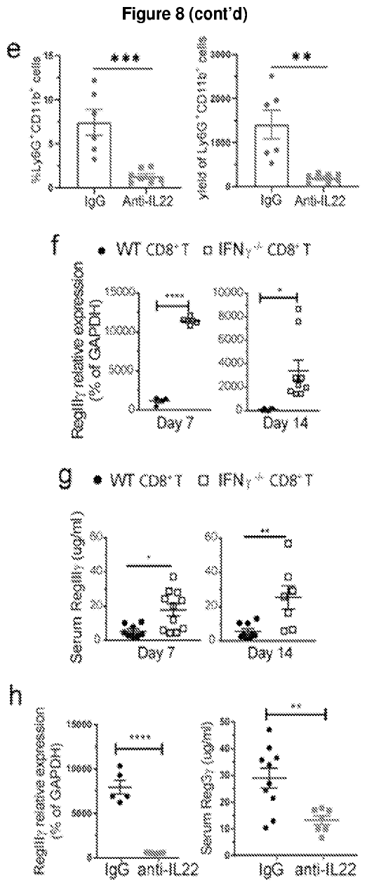

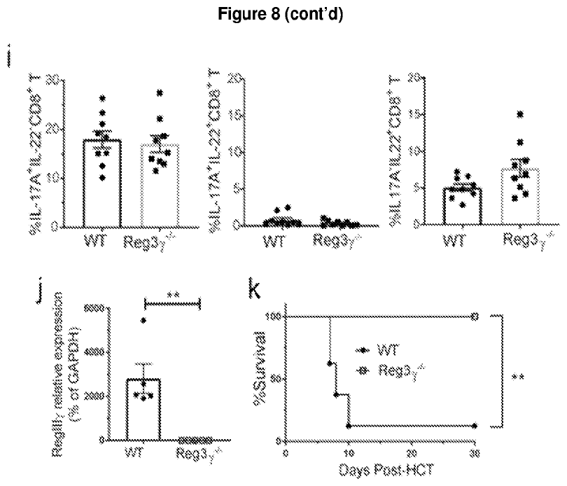

Blockade of mouse interleukin-22 and mouse IFNy in vivo: Anti-mouse IL-22 (Genentech Clone 8E11, mouse IgG1) was administered via intraperitoneal (i.p.) injection at 150 ug every 3 days from day 12 to day 21 after HCT (FIGS. 2 and 4) or every other day from day 0 to day 8 after HCT (FIGS. 8, 9, 12, and 14). Certain control groups also received isotype control IgG1 mAb. Anti-mouse IFNγ (Bio-x-cell, clone R4-6A2) was administered via i.p. injection at 1 mg every day from day 0 to day 5 after HCT. Anti-mouse CD4 (Bio-x-cell, clone GK1.5) was administered via i.p. injection at 500 ug on the day of HCT.

Antibodies, FACS analysis and FACS sorting: Purified depleting anti-mouse CD4 mAb (GK1.5), blocking anti-mouse PD-L1 (10F.9G2), anti-mouse IFNγ mAb (R4-6A2) and mouse IgG (MOPC-21) for in vivo treatment was purchased from Bio X Cell (West Lebanon, NH). Anti-mouse IL-22 mAb (8E11) for in vivo treatment was provided by Genentech (South San Francisco, California). ChromPure Rat IgG (012-000-003) were purchased from Jackson ImmunoResearch Laboratories, Inc. (West Grove, PA, USA). Anti-mouse H2Kb (AF6-88.5.5.3) PE/CY7, anti-mouse TCRβ (H57-597) PE/CY7, anti-mouse CD8α (53-6.7) eflour 450, anti-mouse CD103 (2E7) biotin, anti-mouse CD11b (M1/70) eflour 450, anti-mouse PD-1 (J43) APC, Annexin V PE-CY7, anti-mouse hematopoietic lineage antibody cocktail efluor450, anti-mouse NKP46 (29A1.4) PE/CY7, anti-mouse CD45 (Ly-5) APC, anti-mouse PD-L1 (MIH5) PE, anti-human IFNγ (4S.B3) efluor450, anti-human IL-22 (22URTI) PE and Streptavidin PE/CY7 were purchased from eBioscience; anti-mouse H2Kb (AF6-88.5) FITC, anti-mouse IL-17A (TC11-18H10.1) PE, anti-mouse IL-22 (Poly5164) APC, anti-mouse AHR (4MEJJ) FITC, anti-mouse CD11c (N418) APC/CY7, anti-mouse CD103 (2E7) FITC, anti-mouse CX3CR1 (SA011F11) PE/CY7, anti-mouse CX3CR1 (SA011F11) PE, anti-mouse CD11b (M1/70) Percp-CY5.5, anti-mouse IL10R (1B1.3a) PE, anti-mouse CSF1-R (AFS98) BV711, anti-mouse CD64 (X54-5/7.1) BV605, anti-mouse F4/80 (BM8) BV711, anti-mouse CD127 (A7R34) APC/CY7, anti-mouse MerTK (2B10C42) APC, anti-human CD3 (UCHT1) FITC, anti-human CD4 (SK3) BV605, anti-human CD8 (SK1) APC/CY7 and anti-human IL17A(BL168) APC were purchased from Biolegend; anti-mouse H2Kb (AF6-88.5) BV605, anti-mouse CD90.2 (30-H12) FITC, anti-mouse RORγT (Q31-378) PE and anti-mouse CD8α (53-6.7) BUV395 were purchased from BD Bioscience. Monoclonal anti-Cytokeratin (PCK-26) FITC was purchased from Sigma. Flow cytometry analyses were performed with CyAn Immunocytometry system (DAKO Cytomation, Fort Collins, CO), Attune NxT Flow Cytometer (ThermoFisher Scientific) and BD LSRFortessa (Franklin Lakes, NJ), the resulting data were analyzed with FlowJo software (Tree Star, Ashland, OR). Cell sorting was performed with a BD FACS Aria SORP sorter at the City of Hope FACS facility. The sorted cells were used for transfer experiments.

RNA isolation and real-time reverse transcriptase PCR: The tissue RNA was isolated with the TRIzol Reagent (Life technology) according to the manufacturer's instruction. Real-time RT-PCR was conducted on an ABI 7500 Real-Time PCR system (Applied Biosystems) with primers and power SYBR Green PCR master mix (Applied Biosystems). The samples were normalized to the control housekeeping gene. Real-time RT-PCR analysis of mRNA for Reg3γ, Defensin-α1 and Defensin-α3 was performed as described in previous publications53-, the primers used are as follows: (Reg3γ) 5′-TTCCTGTCCTCCATGATCAAAA-3′ (SEQ ID NO: 1) and 5′-CATCCACCTCTGTTGGGTTCA-3′ (SEQ ID NO: 2); (Defensin-α3) 5′-CCCAGAAGGCTCTTCTCTTC-3′ (SEQ ID NO: 3) and 5′-CAGCGACAGCAGAGTGTGTA-3′ (SEQ ID NO: 4); (Defensin-α1) 5′-CAGGCCGTATCTGTCTCCTT-3′ (SEQ ID NO: 5) and 5′-ATGACCCTTTCTGCAGGTTC-3′ (SEQ ID NO: 6); (GAPDH) 5′-TCACCACCATGGAGAAGGC-3′ (SEQ ID NO: 7) and 5′-GCTAAGCAGTTGGTGGTGCA-3′ (SEQ ID NO: 8); and (Actin) 5′-CGGTTGGCCTTAGGGTTCAGGG-3′ (SEQ ID NO: 9) and 5′-GTG GGCCGCTCTAGGCACCAA-3′ (SEQ ID NO: 10).

ELISA: Reg3γ(antibodies online), ST2 (R&D system) and sTNFR1 (Thermo Scientific) in serum were measured by enzyme-linked immune sorbent assay (ELISA) according to manufacturer's protocol. Serum was collected and spun down at 2000 rpm for 10 min at 4° C. Clear supernatant was collected and stored at −20° C. until ELISA analysis.

Sample collection and DNA extraction: Ileal stool samples were frozen at −80° C. For 16S Miseq sequencing, DNA was extracted with the Power Soil DNA isolation kit (QIAGEN).

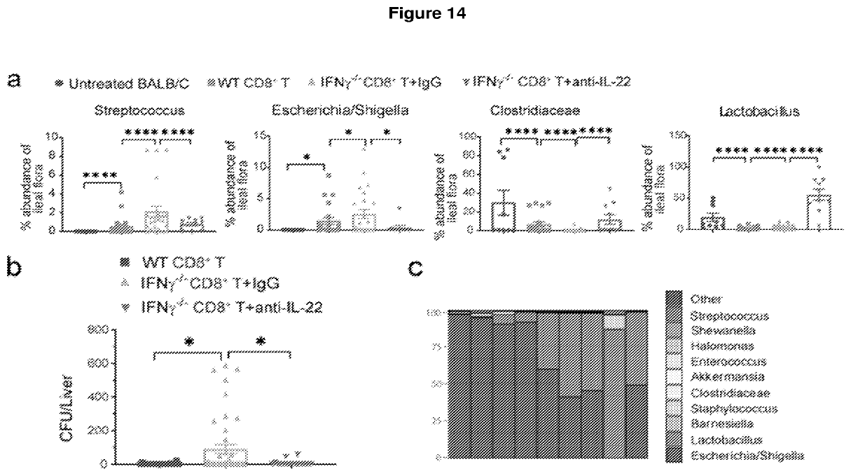

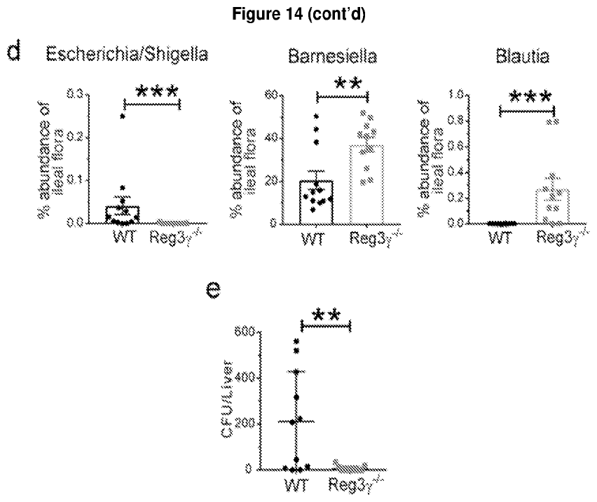

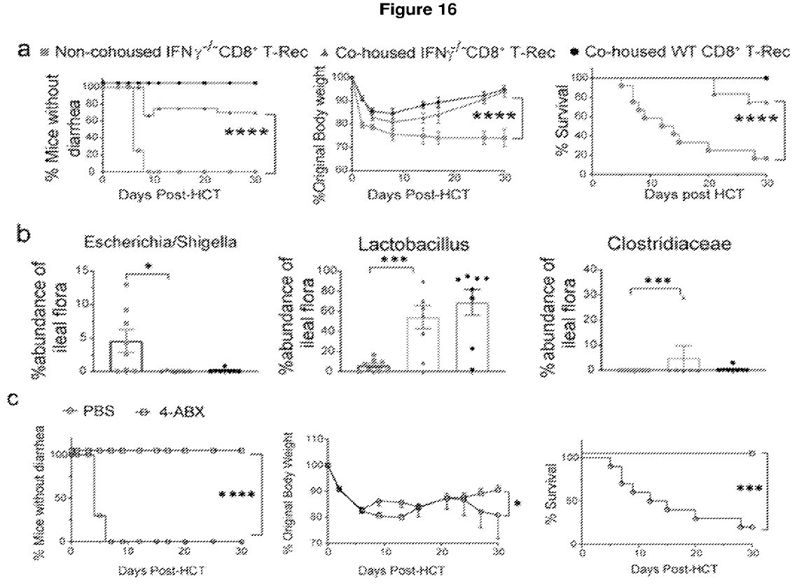

16S rRNA gene amplification and Miseq sequencing and data analysis: For FIGS. 14 and 16, 16S rRNA gene amplification and Miseq sequencing and data analysis, the V4-V5 16S rRNA region were amplified and sequenced using the Illumina MiSeq platform. Duplicate 50-μl PCR reactions were performed, each containing 50 ng of purified DNA, 0.2 mM dNTPs, 1.5 mM MgCl₂, 1.25 U Platinum Taq DNA polymerase, 2.5 μl of 10×PCR buffer, and 0.5 μM of each primer designed to amplify the V4-V5: F (5′-ACACTCTTTCCCTACACGACGCTCTTCCGATCTAYTGGGYDTAAAGNG-3′ (SEQ ID NO: 11)) and R (5′-GTGACTGGAGTTCAGACGTGTGCTCTTCCGATCTCCGTCAATTYHTTTREGT-3′ (SEQ ID NO: 12)). Cycling conditions were 94° C. for 3 minutes, followed by 22 cycles of 94° C. for 50 seconds, 51° C. for 40 seconds, and 72° C. for 1 minute. 72° C. for 5 min is used for the final elongation step. Amplicons were purified using AxyPrep Mag PCR Clean-up kit (Thermo Fisher Scientific). Up to 15 ng of PCR products were carried forward to library preparation using second round PCR. The Illumina primer PCR PE1.0 and index primers were used to allow multiplexing of samples. Eight cycles of enrichment PCR were performed, and final libraries cleaned with AxyPrep Mag PCR Clean-up kit. The library was quantified using ViiA™ 7 Real-Time PCR System (Life Technologies) according to manufacturer's instructions and visualized for size validation on an Agilent 2100 Bioanalyzer (Agilent Technologies) using a high sensitivity DNA assay according to manufacturer's instructions. The sequencing library pool was diluted to 4 nM until run on a MiSeq desktop sequencer (Illumina). 600 cycles chemistry (Illumina) was used according to manufacturer's instructions to run the 6 pM library with 20% PhiX (Illumina), and FASTQ files were used for data analysis. Reads (300 bp paired-end) were merged and then quality-filtered to remove reads with degenerate sites using mothur69, 70 (using the make.contigs and screen.seqs functions, respectively). Reads were then further quality filtered by length (350-375 bp for V4-5). Genus-level assignments per-read were then made using SILVA71 reference sequences in mothur with classify.seqs at 80% confidence.

16S PacBio SMRT Sequencing and data analysis: For FIG. 4 microbial DNAs from 1-3 mg of murine fecal samples from cecum were extracted and purified according to the manufacturer's protocol of EXT3-16S DNA Purification and PCR Amplification Kit of Shoreline Biome (Farmington, CT), and the extended (˜2,500 bp) region, which contains 16S rRNA gene, the adjacent Internally Transcribed Spacer (ITS) and part of the 23S gene, was amplified using barcoded primer sets in the same kit.

For construction of SMRTbell libraries, all reagents were provided by PacBio (Menlo Park, CA) SMRTbell Template Prep Kit 1.0. Equal molar quantities of the amplicons were pooled, and 50 ul of the DNA repair mixture containing 37 ul of pooled DNA, 5 ul of DNA Damage Repair Buffer (10×), 0.5 ul of NAD₊(100×), 5 ul of ATP high (10 mM), 0.5 ul of dNTP (10 mM) and 2 ul of DNA Damage Repair Mix were incubated at 37° C. for 20 minutes. To generate blunt ends of the DNA, 2.5 ul of End Repair Mix (20×) was treated at 25° C. for 5 minutes. 40 ul of the adapter ligation mixture containing 20 ul of blunted ended DNAs, 5 ul of Annealed Blunt Adapter (20 uM), 4 ul of Template Prep Buffer (10×), 2 ul of ATP low (1 mM) and 1 ul of ligase (30 U/ul) was incubated at 25° C. overnight. To degrade failed ligation products, 1 ul of ExoIII (100.0 U/ul) and ExoVII (10.0 U/ul) was treated to the mixture at 37° C. for 1 hour.

To produce polymerase complexes, all reagents were provided by PacBio (Menlo Park, CA) and the reagent concentrations in the protocol were calculated by the Sample Setup module in PacBio SMRT Link (v8.0.0.80529). The Sequencing Primer v2 (1 ul) was diluted by 29 ul of Elution Buffer and the mixture was incubated at 80° C. for 2 minutes. The conditioned sequencing primers were incubated with the library at 20° C. for 1 hour. 1 ul of Sequel Polymerase 3.0 was diluted by 9 ul of Sequel Binding Buffer. The polymerase was further diluted by adding 1 ul of Sequel Binding Buffer into 3.2 ul of diluted polymerase. The diluted polymerase was applied to the library with the sequencing primers, and the mixture was incubated at 30° C. for 1 hour. After purification of the polymerase complexes using AMPure PB Beads, 85 ul of the final loading dilution in the sample plate was loaded into Sequel. The concentration of the sample on the plate was 8 pM and Movie Time was 10 hours and 2 hours of Pre-Extension Time was applied. The primary analyses, including real-time signal processing and base calling were processed by a built-in PacBio Blade Center through Sequel ICS, and result stream directly to SMRT Link (v8.0.0.80529). The CCS reads (>5 Minimum Number of Passes and 0.99 Minimum Predicted Accuracy) were produced by the Circular Consensus Sequences (CCS) module in SMRT Link (v8.0.0.80529). The demultiplexing and the taxonomic classification analysis of the CCS reads were carried out using SBanalyzer (v2.4-2) of Shoreline Biome (Farmington, CT) based on Athena V2 database.

Bacteria culture: Total liver cell suspension was cultured under 5% CO2 in blood agar plates for 24-48 hours at 37° C.

Histological analysis: Tissue specimens were fixed in formalin before embedding in paraffin blocks, sectioned and stained with H&E. Slides were examined at 100× or 200× magnification and visualized with a Zeiss Observer II. Each segment of colon was given a score of 0-4: grade 0, no significant changes; grade 1, minimal scattered mucosal inflammatory cell infiltrates, with or without minimal epithelial hyperplasia; grade 2, mild scattered to diffuse inflammatory cell infiltrates, sometimes extending into the submucosa and associated with erosions, with mild to moderate epithelial hyperplasia and mild to moderate mucin depletion from goblet cells; grade 3, moderate inflammatory cell infiltrates that were sometimes transmural, with moderate to severe epithelial hyperplasia and mucin depletion; and grade 4, marked inflammatory cell infiltrates that were often transmural and associated with crypt abscesses and occasional ulceration, with marked epithelial hyperplasia, mucin depletion, and loss of intestinal glands. For Paneth cell quantification, a total of 9 pictures from 3 different locations of the H&E-stained slides from one mouse were taken under 200× magnification, and total Paneth cell and crypt numbers were counted, Paneth cell numbers per crypt are shown.

Statistics Analysis: Student's unpaired t-test was used to compare two groups when data were normally distributed. Mann-Whitney test was used to compare two groups when data were not normally distributed. For comparing more than two groups, Kruskal-Wallis test with Dunn's multiple comparisons test was used was used to compare two groups when data were not normally distributed. One-way ANOVA with Tukey's multiple comparisons test, with the Greenhouse-Geisser correction or Student's t test corrected for multiple comparisons using the Holm-Sidak method were used to compare means in paired samples. Ordinary one-way ANOVA with Tukey's correction for multiple comparisons, two-way ANOVA with Tukey's or Sidak's correction for multiple comparisons was used to compare means in unpaired samples. Log-rank test was used for survival comparisons. Nonlinear regression (curve fit) was used for body weight and diarrhea comparisons. All statistical analyses were performed using GraphPad Prism 8. P values<0.05 were considered statistically significant.

Data availability: 16S PacBio SMRT Sequencing data has been deposited in the SRA database under the accession number PRJNA667464. 16S Miseq data have been deposited in GEO database under the accession number GSE159419.

### Example 2. Establishment of a Murine Model for Steroid-Resistant Acute Gut GVHD

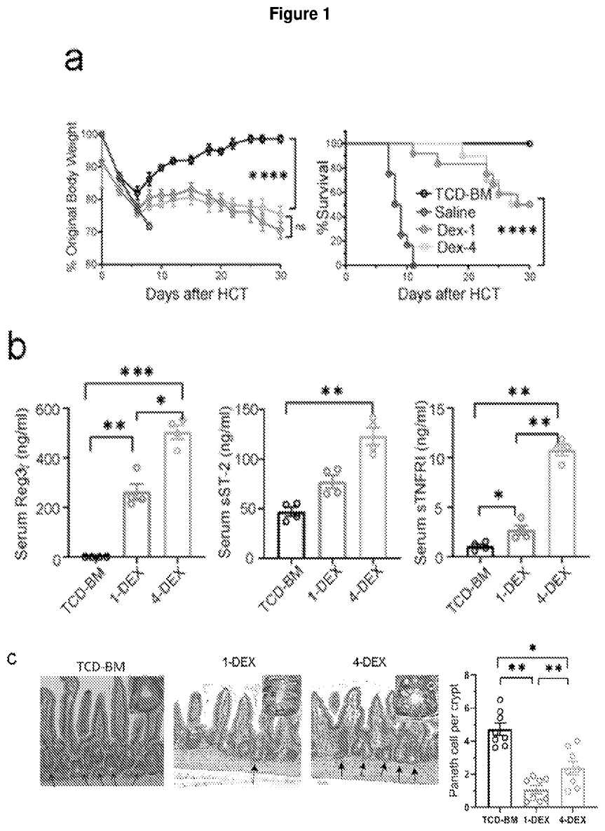

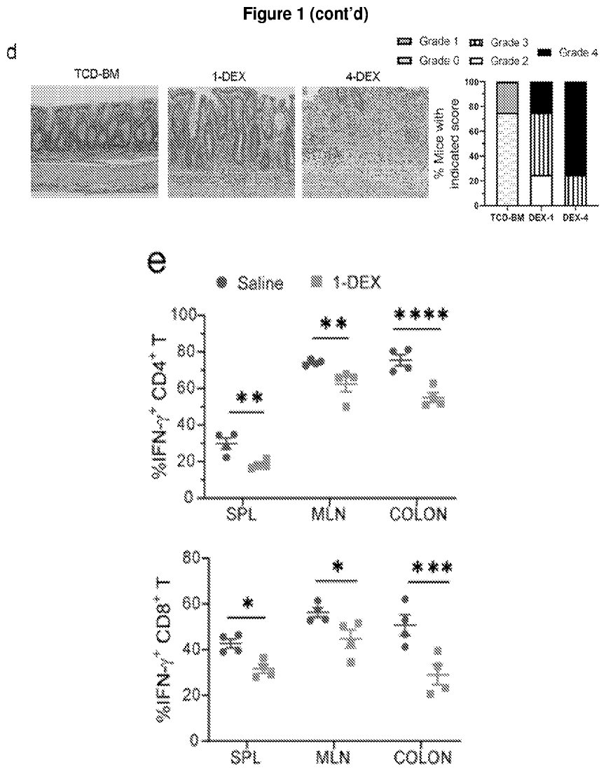

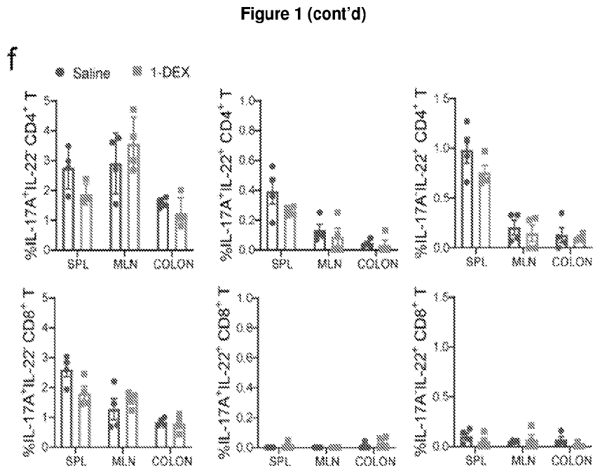

A model of SR-gut-aGVHD in mice was established. Lethal TBI-conditioned BALB/c mice (H-2^{d}) were given spleen (SPL) cells containing 1.5×10⁶ T cells together with bone marrow (BM) cells (2.5×10⁶) from MHC-mismatched C57BL/6 (H-2^{b}) donors. Three days after HCT, recipients were given a single injection of dexamethasone (DEX) at 5 mg/Kg. This treatment ameliorated gut-aGVHD as indicated by prevention of diarrhea, reduction of bodyweight loss, and enabled recipients to survive for up to 20 days after HCT with mild clinical GVHD. In contrast, controls treated with saline all (12/12) died by day 11 with diarrhea and severe bodyweight loss (FIG. 1a). After day 20, however, the DEX-treated mice developed bodyweight loss and began to die. Additional DEX administration on days 10, 15 and 20 (total 4-DEX) did not prevent this deterioration or improve survival as compared to single DEX treatment on day 3 (FIG. 1a). High serum concentration of Reg3α, ST2 and sTNFR has been associated with SR-gut-aGVHD in patients²⁷. The 4-DEX-treated recipients also showed marked increase in serum Reg3γ, ST2, and sTNFR as compared with non-GVHD or 1-DEX-treated GVHD recipients (FIG. 1b). At 30 days after HCT, 4-DEX-treated recipients had higher numbers of Paneth cells in the small intestine but more severe GVHD in the colon compared to 1-DEX-treated recipients (FIGS. 1c and 1d). These results indicate that gut-aGVHD in recipients given 4-DEX-treatment evolved to SR-gut-aGVHD.

### Example 3. SR-gut-aGVHD is Associated with Expansion of AHR⁺Th/Tc22 Cells

In view of the prior reports that steroids augment Th/Tc1 apoptosis²⁹ and reduce tissue release of TGF-β but not IL-6^{36,37} and that TGF-β and IL-6 reciprocally regulate differentiation of Th17 and Th22³⁸⁻⁴⁹, the changes of Th/Tc1, Th/Tc17 and Th/Tc22 subsets in recipients with or without DEX treatment were analyzed. Because the saline-treated control mice all died ˜10 days after HCT, the T cell subsets in the SPL, mesenteric lymph nodes (MLN) and colon tissues were first compared at 7 days after HCT. As compared to saline controls, DEX treatment on day 3 significantly reduced the percentages of IFN-γ⁺Th/Tc1 cells in SPL, MLN and colon tissues (FIG. 1e). At the same time, the numbers of Th/Tc17 cells were very low, even in the recipients treated with DEX, there were only 1-5% IL-17A⁺IL-22⁻CD4⁺Th17 cells and IL-17A⁺IL-22⁻CD8⁺Tc17 cells among CD4⁺and CD8⁺T cells (FIG. 1f). The percentages of IL-17A⁺IL-22⁺CD4⁺T cells were less than 0.5%, and IL-17A⁺IL-22⁺CD8⁺T cells were essentially undetectable. The percentage of IL-17A⁻IL-22⁺CD4⁺was ˜0.5-1% and IL-17A⁻IL-22⁺CD8⁺T cells were barely detectable (FIG. 1f). These results indicate that DEX-treatment early after HCT mainly reduces Th/Tc1 expansion, with little impact on Th/Tc17 or Th/Tc22.

To analyze the T cell subsets in SR-gut-aGVHD, recipients were transplanted with spleen cells containing 0.75×10⁶ instead of 1.5×10⁶ T cells to allow the saline control group to survive for more than 25 days after HCT. On day 25, as compared to saline treatment, 1-DEX and 4-DEX treatments both decreased the numbers of donor-type IL-17A⁺IL-22⁻CD4⁺and CD8⁺T cells in the MLN, although the reduction in the spleen was variable, and no significant difference between 1-DEX and 4-DEX treatment (FIG. 2a). DEX treatment did not change the numbers of IL-17A⁺IL-22⁺CD4⁺or CD8⁺T cells in the spleen or MLN (FIG. 2b). Interestingly, 4-DEX treatment significantly increased both the percentages and the yields of IL-17A⁻IL-22⁺CD4⁺and CD8⁺T cells, in both the spleen and MLN, as compared to 1-DEX treatment or saline (FIG. 2c).

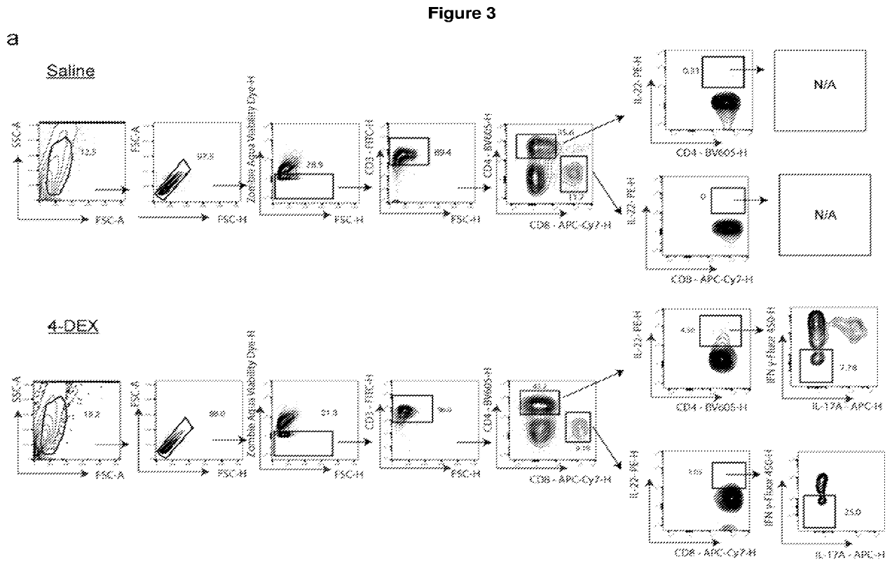

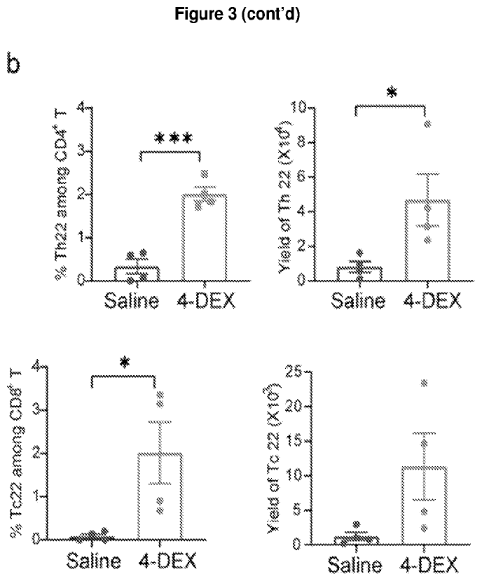

IL-17A⁺IL-22⁻CD4⁺T cells were RORγt⁺AHR⁻Th17 cells, while only small portion of IL-17A⁺IL-22⁻CD8⁺T cells were RORγt⁺AHR⁻Tc17 cells; in contrast, IL-17A⁻IL-22⁺CD4⁺and CD8⁺T cells were both AHR⁺RORγt⁻Th/Tc22 cells (FIG. 2d). Consistent with day 7 results, 4-DEX-treatment significantly reduced the percentage of IFN-γ⁺ Th/Tc1 cells in gut tissues (FIG. 2e). Additionally, 3 healthy human PBMCs were tested. PBMCs from one of these donors induced SR-gut-aGVHD with 4-DEX treatment. The Xeno-SR-gut-aGVHD induced by these cells was also associated with expansion of IL-17A⁻IL-22⁺CD4⁺and CD8⁺T cells in the colon tissue (FIG. 3). These results indicate that prolonged steroid treatment augments expansion of AHR⁺RORγt⁻IL-17A⁻IL-22⁺CD4⁺and CD8⁺Th/Tc22 cells while reducing RORγt⁺AHR⁻IL-17A⁺IL-22⁻Th/Tc17 cells, indicating that SR-gut-aGVHD pathogenesis is associated with preferential expansion of AHR⁺Th/Tc22 cells in the colon.

### Example 4. IL-22 from Th/Tc22 Cells is Required for Induction of SR-gut-aGVHD

Whether expansion of Th/Tc22 cells caused SR-gut-aGVHD was tested. BALB/c recipients engrafted with 1.5×10⁶ splenic T cells and TCD-BM cells from WT C57BL/6 donors were treated with 4-DEX. The recipients were treated with anti-IL-22 or control mouse-IgG1 at a dose of 150 lag every three days, from day 12 to day 21 after HCT. To further validate the role of IL-22 from donor-type T cells, in the same experiment, a group of BALB/c recipients were engrafted with 1.5×10⁶ splenic T cells from IL-22^{-/-}C57BL/6 donors with TCD-BM from WT C57BL/6 donors and were treated with 4-DEX. At day 25 after HCT, recipients were sacrificed for evaluation of gut-aGVHD. Neutralizing IL-22 with anti-IL-22 mAb and preventing IL-22 production by using IL-22^{-/-}T cells both markedly reduced the severity of gut-aGVHD on day 25 after HCT when compared to the positive control (FIG. 2f). These results indicate that IL-22 from donor-type T cells plays a critical role in the pathogenesis of SR-gut-aGVHD.

The 4-DEX-treatment expanded only IL-17A⁻IL-22⁺Th/Tc22 cells, while IL-17A⁺IL-22⁺CD4⁺and CD8⁺Th/Tc17 cells were hardly detectable (FIGS. 2b-2d). To determine whether IL-22 from RORγt⁺ Th/Tc17 cells is required for induction of SR-gut-aGVHD, 4-DEX-treated GVHD recipients given 1.5×10⁶ splenic T cells from WT or RORγt^{-/-}C57BL/6 donors were compared with TCD-BM from WT C57BL/6 donors. RORγt deficiency in donor T cells reduced the percentage but not numbers of IL-17A⁻IL-22⁺Th22 cells in the MLN and reduced both the percentage and yield of IL-17A⁻IL-22⁺Tc22 cells in the MLN on day 25 after HCT (FIG. 2g). The reduction of Th/Tc22 cells with RORγt^{-/-}T cells is consistent with previous reports that RORγt augments AHR⁺Th22 differentiation¹⁷. RORγt deficiency in donor T cells, however, did not significantly change the severity of SR-gut-aGVHD (FIG. 2h). These results indicate that IL-22 from Th/Tc17 cells is not required for SR-gut-aGVHD.

### Example 5. IL-22 from Th/Tc22 Cells Causes Dysbiosis and Bacteria Translocation in SR-gut-aGVHD Recipients

Under inflammatory conditions, IL-22 augments pathogen colonization and dysbiosis in gut tissues12-. Whether SR-gut-aGVHD mediated by IL-22 from Th/Tc22 was associated with dysbiosis was tested. Day 25 after HCT, feces from the ileum of 1-DEX-treated gut-aGVHD recipients and 4-DEX-treated SR-gut-aGVHD recipients given WT-T cells, 4-DEX-treated non-SR-gut-aGVHD recipients given IL-22^{-/-}T cells, and non-GVHD recipients given TCD-BM alone were analyzed for 16S ribosomal RNA sequences. As compared with 1-DEX-treated gut-aGVHD recipients, 4-DEX-treated SR-gut-aGVHD recipients showed significant loss of bacterial diversity, and IL-22-deficiency in donor T cells restored bacterial diversity, as judged by the numbers of species, Shannon index and Fisher index (FIG. 4a). As compared with non-GVHD recipients, 1-DEX-treated gut-aGVHD recipients showed slight but not significant reduction in bacterial diversity (FIG. 4a). Principal coordinate analysis also showed that bacteria from 4-DEX-treated SR-gut-aGVHD were distinguishable from those of 1-DEX-treated gut-aGVHD recipients or non-GVHD recipient, and IL-22-deficiency in donor T cells reduced the difference (FIG. 4b).

Further analyzing bacterial subpopulations at species level showed that 4-DEX-treated SR-gut-aGVHD recipients had marked expansion of Enterococcus_sp._FDAARGOS_553 and that IL-22 deficiency in donor T cells prevented this effect; although the differences in Lactobacillus murinus and E. Coli were not statistically significant (FIGS. 4c and 4d). As a further measure of dysbiosis, liver tissue suspension was cultured for bacteria colony formation. As compared with 1-DEX-treated gut-aGVHD or non-GVHD recipients, 4-DEX-treated SR-gut-aGVHD recipients had a marked increase in bacterial colony formation, and IL-22 deficiency in donor T cells prevented this effect (FIG. 4e). These results indicate that donor-type Th/Tc22-mediated SR-gut-aGVHD is associated with dysbiosis and enhanced bacterial translocation into the liver, and IL-22 deficiency in donor T cells prevents the induction of SR-gut-aGVHD.

### Example 6. Gut-aGVHD Induced by IFN-γ^{-/-}Donor CD8⁺T Cells is Associated with Expansion of Tc17 and Tc22 Cells

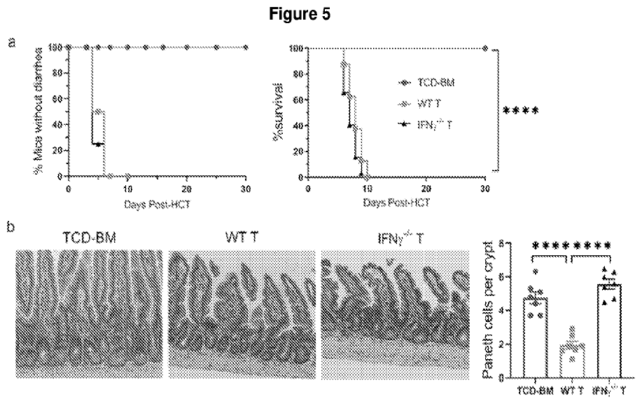

Although splenic T cells from WT and IFN-γ^{-/-}C57BL/6 donors both induced severe gut-aGVHD, IFN-γ^{-/-}T cell had no effect on small intestine Paneth cells (FIG. 5). It was reported that WT donor CD8⁺T cells did not cause gut-aGVHD in the absence of donor CD4⁺T cells⁴¹; it was also reported that IFN-γ was required for preventing GVHD mediated by CD8⁺T cells⁴². IFN-γ^{-/-}donor CD8⁺T cells alone induced severe gut-aGVHD. Since SR-gut-aGVHD mediated by expansion of IL-22⁺Th/Tc22 cells was associated with reduction of IFN-γ⁺ Th/Tc1 (FIGS. 1 & 2), it was hypothesized that gut-aGVHD induced by IFN-γ^{-/-}CD8⁺T cells could reflect the pathogenesis in SR-gut-aGVHD.

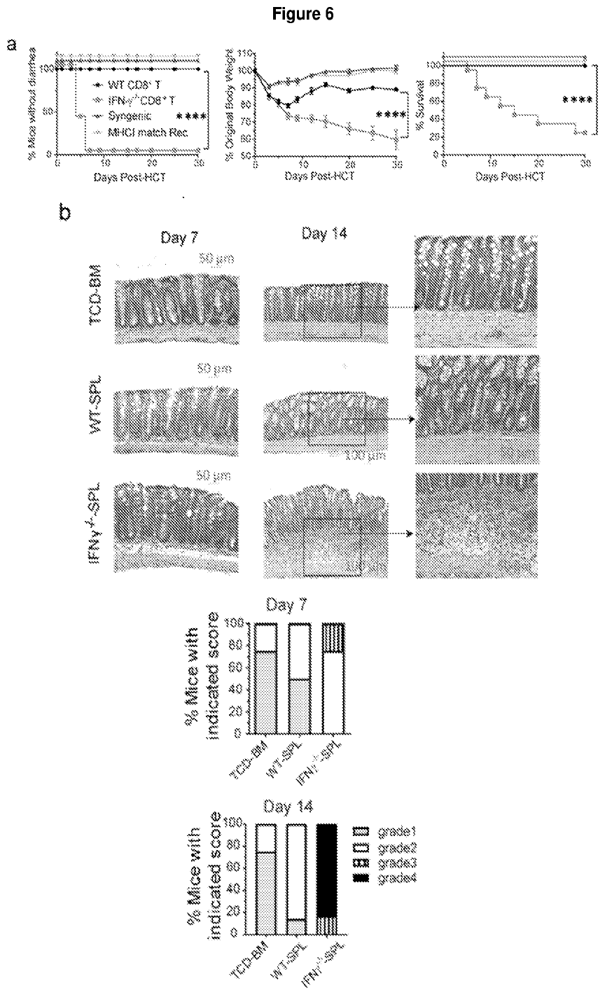

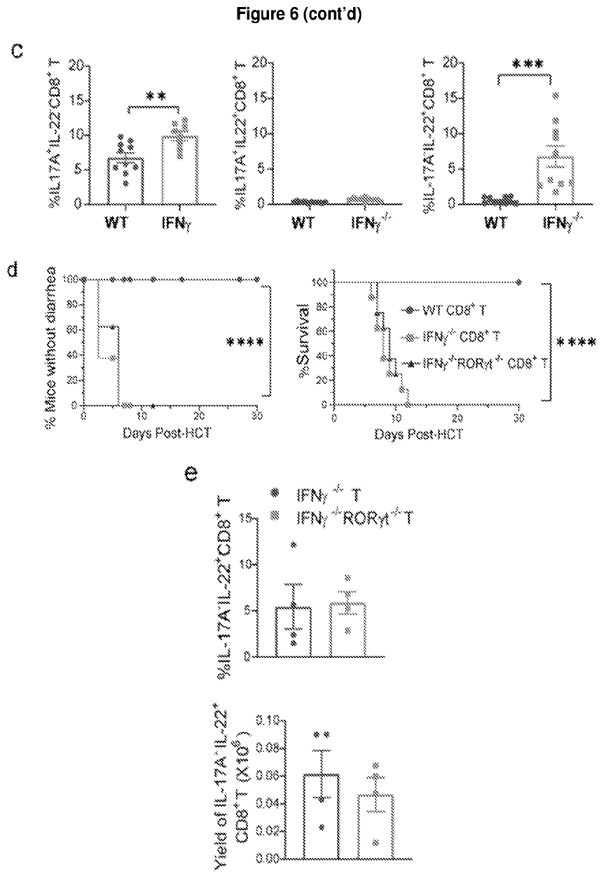

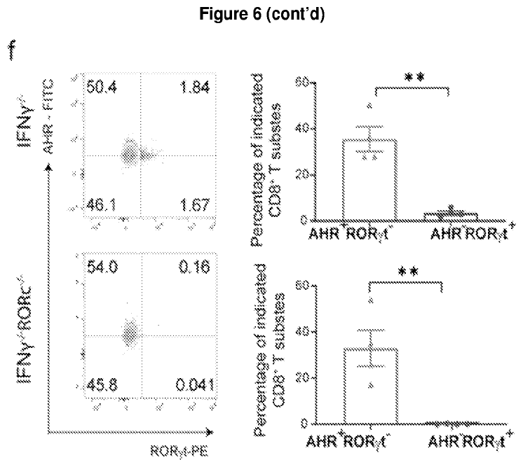

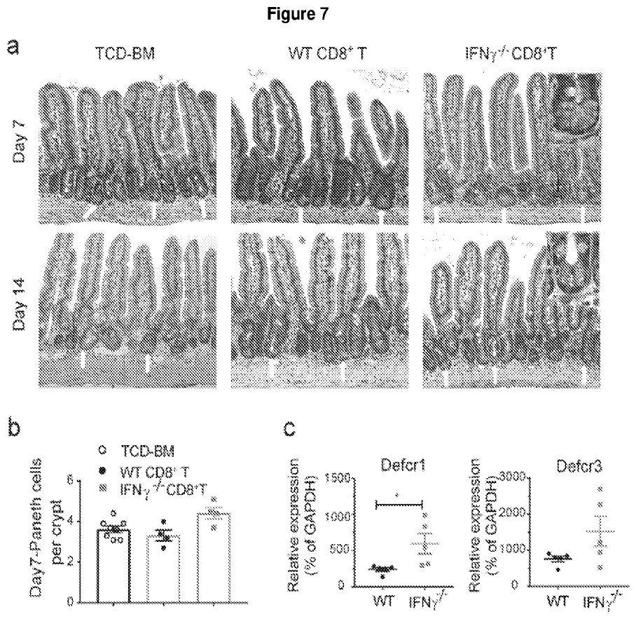

Accordingly, lethal TBI-conditioned BALB/c recipients were engrafted with spleen cells containing 1.5×10⁶ T cells and bone marrow cells (2.5×10⁶) from WT or IFN-γ^{-/-}C57BL/6 donors, and the recipients were given a single injection of anti-CD4 mAb to deplete the CD4⁺T cells41-. Under these conditions, recipients given IFN-γ^{-/-}CD8⁺T cells developed aGVHD, but recipients given WT-CD8⁺T cells did not (FIG. 6a). IFN-γ^{-/-}CD8⁺T cells did not induce disease in syngeneic or MHC I-matched recipients (FIG. 6a). Recipients given IFN-γ^{-/-}-SPL cells developed severe infiltration in the colonic submucosa from days 7 to 14 after HCT (FIG. 6b), with little damage in the epithelial cells or Paneth cells in the ileum (FIGS. 7a, 7b). Expression of Defensin-α1 and Defensin-α3 mRNA in the ileal tissue of the recipients was higher in recipients given IFN-γ^{-/-}-SPL cells than in those given WT-SPL cells (FIG. 7c). At 7 days after HCT, the percentages of IL-17A⁺IL-22⁻and IL-17A⁻IL-22⁺CD8⁺T cells in MLN were higher in recipients given IFN-γ^{-/-}-SPL cells than in those given WT-spleen cells; and percentages of IL-17A⁺IL-22⁺cells were very low in both groups (FIG. 6c).

In further experiments, sorted CD8⁺T cells (1.5×10⁶) from WT, IFN-γ^{-/-}or IFN-γ^{-/-}RORγt^{-/-}were co-transplanted with WT-TCD-BM cells (2.5×10⁶). WT CD8⁺T cells induced little GVHD, IFN-γ^{-/-}and IFN-γ^{-/-}RORγt^{-/-}T cells both induced severe lethal gut-aGVHD with diarrhea (FIG. 6d). There was no significant difference in the percentage and yield of IL-17A⁻IL-22⁺CD8⁺T cells in the MLN of the recipients given IFN-γ^{-/-}or IFN-γ^{-/-}RORγt^{-/-}T cells (FIG. 6e), and they were AHR⁺RORγt^{-/-Tc}22 cells (FIG. 6f). These results indicate that gut-aGVHD-induced by IFN-γ^{-/-}donor CD8⁺T cells is associated with expansion of IL-17A⁺IL-22⁻Tc17 and AHR⁺IL-17A⁻IL-22⁺Tc22 cells.

### Example 7. IL-22 from Tc22 but not IL-17A from Tc17 is Required for gut-aGVHD Induced by IFN-γ^{-/-}CD8⁺T cells

Since gut-aGVHD induced by IFN-γ^{-/-}CD8⁺T cells was associated with expansion of IL-17A⁺IL-22⁻Tc17 and IL-17A⁻IL-22⁺Tc22 cells (FIG. 6c), whether IL-17A is required for gut-aGVHD pathogenesis was tested by comparing CD8⁺T cells from IFN-γ^{-/-}and IFN-γ^{-/-}IL-17A^{-/-}donors. Recipients were engrafted with CD8⁺T cells and TCD-BM cells from IFN-γ^{-/-}or IFN-γ^{-/-}IL-17^{-/-}C57BL/6 donors. The non-GVHD recipients given IFN-γ^{-/-}or IFN-γ^{-/-}IL-17A^{-/-}TCD-BM cells alone were combined into a TCD-BM group (FIG. 8a). Recipients given IFN-γ^{-/-}or IFN-γ/IL-17A^{-/-}CD8⁺T cells both developed diarrhea, body weight-loss, and most of them died within 30 days after HCT, with no difference between the two groups (FIG. 8a). The percentages of IL-17A^{-/-}IL-22⁺CD8⁺T cells in the MLN at 7 days after HCT did not differ between the 2 groups (FIG. 8b). These results indicate that IL-17A produced by Tc17 cells is not required for gut-aGVHD induced by IFN-γ^{-/-}CD8⁺T cells.

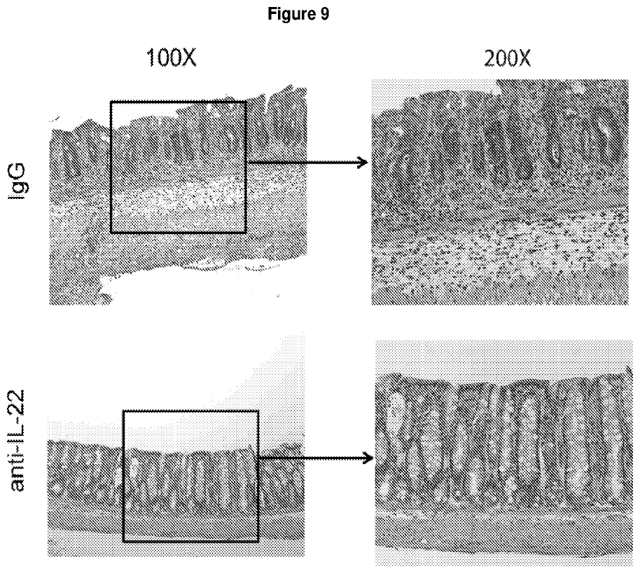

To evaluate the role of IL-22, recipients given IFN-γ^{-/-}CD8⁺T cells were treated with anti-IL-22 or control mouse-IgG1 at 150 μg every other day, until day 8 after HCT. Anti-IL-22 treatment effectively prevented diarrhea in all recipients, and the recipients all survived for more than 30 days (FIG. 8c) with little colon tissue damage (FIG. 9). Anti-IL-22 treatment did not change the percentages of IL-17A⁺IL-22⁻or IL-17A⁻IL-22⁺subsets in the MLN of recipients given IFN-γ^{-/-}CD8⁺T cells (FIG. 8d). Neutrophil infiltration plays an important role in gut-aGVHD damage^{43,44}, and IL-22 from T cells attracted neutrophil into tumor tissues⁴⁵. Consistently, neutralizing IL-22 markedly reduced the percentage and numbers of Ly6G⁺CD11b⁺neutrophils in the colon tissue of gut-aGVHD recipients (FIG. 8e).

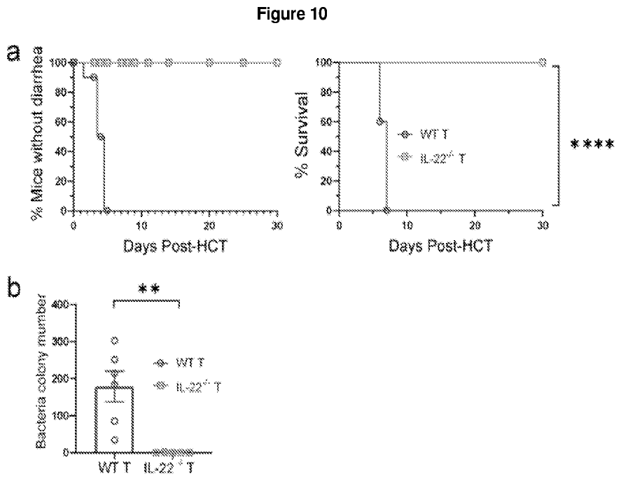

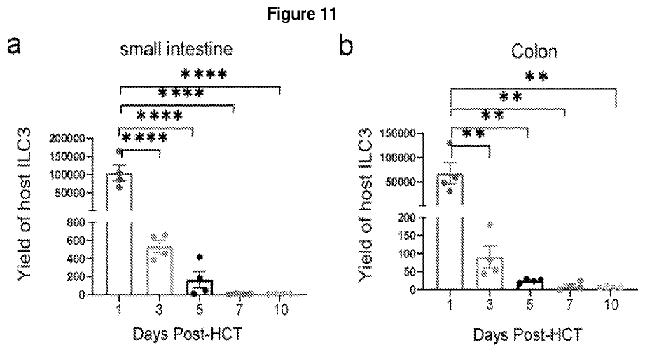

In BALB/c recipients treated with neutralizing antibody against IFN-γduring the first 5 days after HCT, WT CD8⁺T cells induced severe gut-aGVHD and bacterial translocation but IL-22^{-/-}CD8⁺T cells did not (FIGS. 10a, 10b), indicating IL-22 from donor Tc22 cells is required for pathogenesis. Recipient ILC3 in the small intestine and colon were eliminated by 5-7 days after HCT before gut-aGVHD onset (FIG. 11), suggesting they are not required for pathogenesis. Therefore, in the absence of IFN-γ, IL-22 from donor-type Tc22 cells plays a critical role in gut-aGVHD induced by CD8⁺T cells.

### Example 8. Tc22 Differentiation from Alloreactive IFN-γ^{-/-}CD8⁺T Cells Requires Tissue-Damage by Conditioning

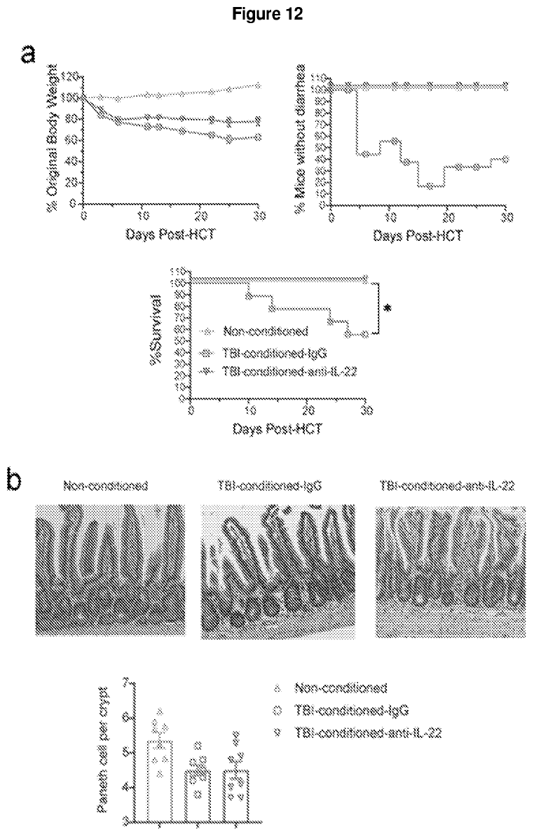

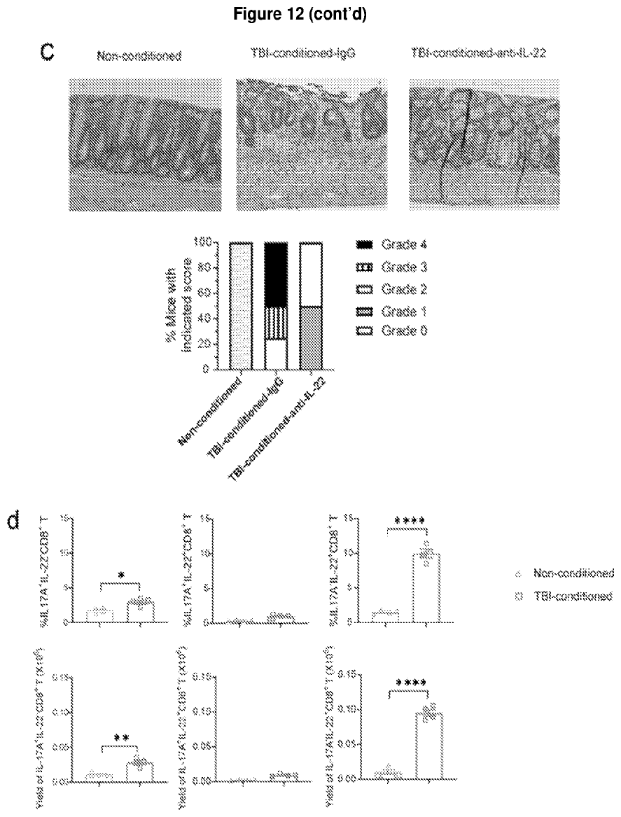

Tissue damage from the conditioning regimen before HCT results in production of proinflammatory cytokines such as IL-6 and IL-1β⁴⁶. Since IL-6 plays an important in augmenting Th/Tc22 differentiation^{40,47}, whether TBI is required for induction of gut-aGVHD by IFN-γ^{-/-}CD8⁺T cells was tested. To avoid rejection of donor cells in non-conditioned recipients, a parent into F1 HCT model using C57BL/6 (H-2^{b}) donors and C57BL/6×BALB/c (H-2^{b/d}) F1 (CB6F1) recipients were used. CD4⁺T-depleted spleen cells containing 2×10⁶ CD8⁺T cells from IFN-γ^{-/-} donor and TCD-BM (2.5×10⁶) from WT donor were transplanted into lethal TBI-conditioned or non-conditioned CB6F1 recipients. In addition, the lethal TBI-conditioned recipients were treated with anti-IL-22 mAb or mouse IgG1. IFN-γ^{-/-}CD8⁺T cells induced gut-aGVHD in the colon but not in the small intestine of lethal TBI-conditioned CB6F1 recipients, and the disease was prevented by anti-IL-22-treatment. No evidence of gut-aGVHD was apparent in non-conditioned recipients (FIGS. 12a-12c). TBI-conditioning also augmented expansion of the IL-17A⁺IL-22⁻and especially the IL-17A⁻IL-22⁺CD8⁺T cell subsets in the MLN of gut-aGVHD recipients (FIG. 12d). Taken together, alloreactive Tc22 differentiation from IFN-γ^{-/-}CD8⁺T cells requires tissue damage by conditioning before HCT.

### Example 9. IL-22 from Tc22 Cells Induces gut-aGVHD via Host-Tissue Production of Reg3γ

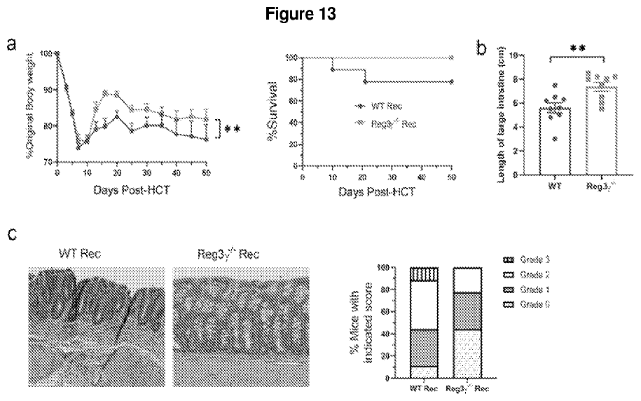

IL-22 can augment Paneth cell and intestinal epithelial cell production of Reg3γ^{4,48}. Consistently, expression of Reg3γmRNA in ileal tissue and serum Reg3γwas higher in recipients given IFN-γ^{-/-}CD8⁺T cells than in those given WT CD8⁺T cells on days 7 and 14 after HCT (FIGS. 8f and 8g). Anti-IL-22 treatment markedly reduced ileal tissue expression of Reg3γmRNA and serum concentrations of Reg3γ(FIG. 8h). Therefore, whether elevation of Reg3γwas required for gut-aGVHD induced by IFN-γ^{-/-}donor CD8⁺T cells was tested by transplanting IFN-γ^{-/-}CD8⁺T cells from BALB/c donors into WT or Reg3γ^{-/-}C57BL/6 recipients. Tc22 expansion did not differ between WT and Reg3γ^{-/-}recipients (FIG. 8i). Expression of Reg3γmRNA in ileal tissue was detected in WT but not Reg3γ^{-/-}recipients (FIG. 8j). WT recipients showed gut-aGVHD, and nearly 90% (7/8) died by 10 days after HCT, but Reg3γ^{-/-}recipients showed no signs of gut-aGVHD, and all survived for more than 30 days (FIG. 8k). In addition, WT and Reg3γ^{-/-}C57BL/6 recipients were treated with 4-DEX after HCT, and significant reduction of gut-aGVHD in Reg3γ^{-/-}recipients was observed (FIG. 13). Therefore, IL-22 from donor Tc22 cells augments host tissue production of Reg3γ, and Tc22 induction of gut-aGVHD is Reg3γ-dependent.

### Example 10. IL-22 from Tc22 Cells Causes Dysbiosis via Reg3γ

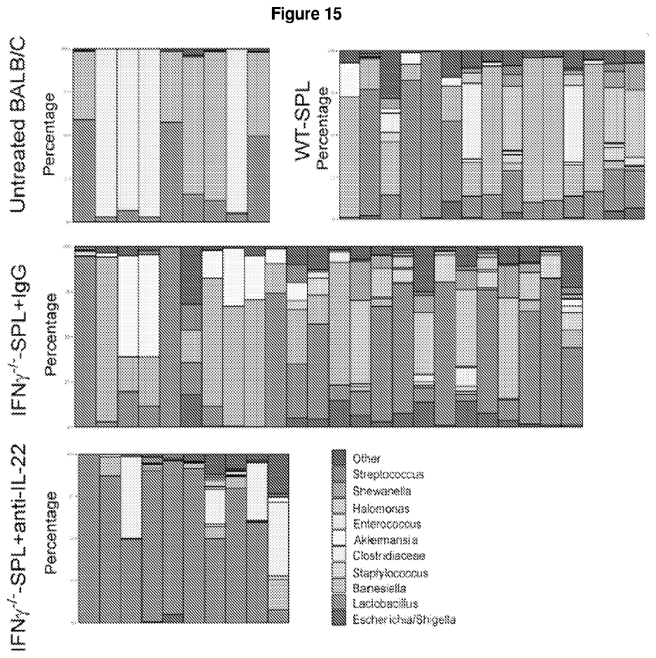

IL-22 could cause dysbiosis enhanced by Reg3γunder inflammatory conditions12-. Therefore, 16S ribosomal RNA sequences 6 was analyzed to evaluate the impact of IL-22 and the associated increased production of Reg3γon microbiota profiles in the ileum of recipients given donor splenic IFN-γ^{-/-}-CD8⁺T cells. At 6 days after HCT, the microbiota profile of gut-aGVHD-free recipients given WT CD8⁺T cells had some differences when compared to untreated BALB/c mice, with increased prevalence of Streptococcus and E. coli (FIGS. 14a and 15), although they did not show clinical signs of gut-aGVHD (FIG. 6a). In contrast, the profiles of gut-aGVHD recipients given IFN-γ^{-/-}CD8⁺T cells showed marked changes, with a lower prevalence of protective Clostridiaceae and a higher prevalence of pathogenic Streptococcus and E. coli (FIGS. 14a and 15). Prevention of gut-aGVHD with anti-IL-22 treatment was associated with a higher prevalence of protective Clostridiaceae and Lactobacillus as well as a lower prevalence of pathogenic Streptococcus, and E. coli. (FIGS. 14a and 15). Additionally, cultures of liver tissue suspension from GVHD-free recipients of WT CD8⁺T cells showed very little bacterial growth, but 37% (17/45) cultures from recipients of IFN-γ^{-/-}CD8⁺T cells showed exuberant bacterial growth, and anti-IL-22-treatment markedly reduced the frequencies of bacterial growth (FIG. 14b). Bacteria in the liver tissue cultures from recipients given IFN-γ^{-/-}CD8⁺T cells were predominantly E. coli with some Lactobacillus and other strains (FIG. 14c).

Finally, the prevalence of protective Bamesiella and Blautia was higher in gut-aGVHD-free Reg3γ^{-/-}recipients than in gut-aGVHD WT recipients, while the prevalence of pathogenic E. coli was lower (FIG. 14d). Bacterial growth frequency was also lower in the liver tissue cultures from Reg3γ^{-/-}recipients (FIG. 14e). These results indicate that IL-22 from Tc22 cells causes dysbiosis by augmenting Reg3γproduction.

### Example 11. Dysbiosis is Required for Induction of gut-aGVHD-Mediated by Tc22-Derived from IFN-γ^{-/-}CD8⁺T Cells

To validate the impact of dysbiosis on induction of gut-aGVHD, recipients of IFN-γ^{-/-}CD8⁺T cells were housed separately or together with recipients of WT CD8⁺T cells at a ratio of 2:3 in cages of 5 mice. Recipients of IFN-γ^{-/-}CD8⁺T cells housed separately all developed diarrhea and bodyweight-loss, and 83% (10/12) died by 30 days after HCT (FIG. 16a). In contrast, recipients of IFN-γ^{-/-}CD8⁺T co-housed with non-GVHD recipients of WT CD8⁺T cells showed significant reduction in gut-aGVHD severity with reduction of diarrhea and higher body weight and survival (FIG. 16a) and lower prevalence of E. coli but higher prevalence of Lactobacillus (FIG. 16b). These results suggest that the microbiome from non-GVHD recipients can ameliorate gut-aGVHD in recipients with dysbiosis.

The role of dysbiosis in gut-aGVHD pathogenesis was further evaluated by eliminating intestinal bacteria. Recipients of IFN-γ^{-/-}CD8⁺T cells were given a daily gavage of four antibiotics, including ampicillin (1 g/L), neomycin (1 g/L), metronidazole (1 g/L), and vancomycin (0.5 g/L) (4ABX)⁴⁹. Consistent with a previous report⁵⁰, bacteria could not be detected in the feces from ileum on day 6 by V4-V5 16S amplification. While PBS control mice all developed diarrhea, and 80% died within 30 days after HCT, the 4-ABX treatment completely prevented diarrhea, and all recipients survived for more than 30 days (FIG. 16c). These results indicate that dysbiosis is required for induction of gut-aGVHD mediated by IFN-γ^{-/-}CD8⁺T cells.

### Example 12. Depletion of Donor-Type CX3CR1hi MNP is Associated with Gut-aGVHD Mediated by IFN-γ^{-/-}Donor CD8⁺T Cells

Bacterial translocated into the liver in gut-aGVHD recipients of IFN-γ^{-/-}CD8⁺T cells but not in non-GVHD recipients of WT CD8⁺T cells, although both showed an increased prevalence of E. coli (FIG. 14a). In addition, recipients of WT CD8⁺T cells did not show signs of gut-aGVHD when co-housed with recipients of IFN-γ^{-/-}CD8⁺T cells (FIG. 16a), suggesting a protective mechanism in those mice. Therefore, abnormalities other than dysbiosis may also contribute to the induction of gut-aGVHD in recipients of IFN-γ^{-/-}CD8⁺T cells.

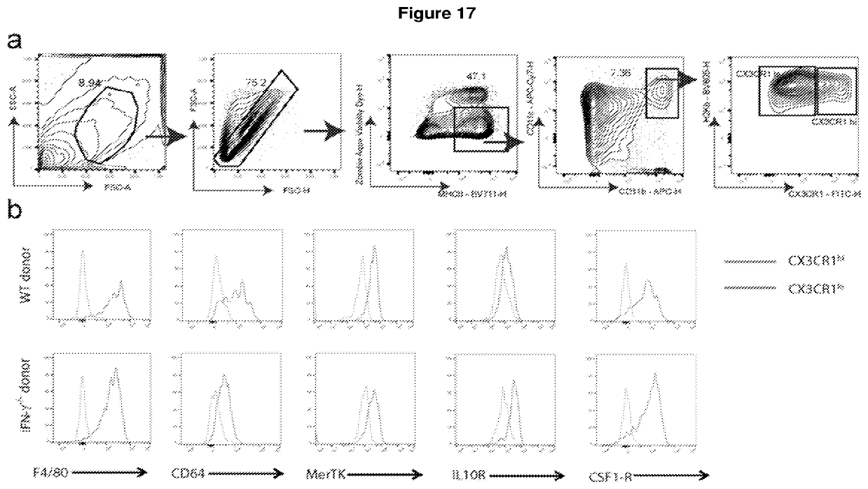

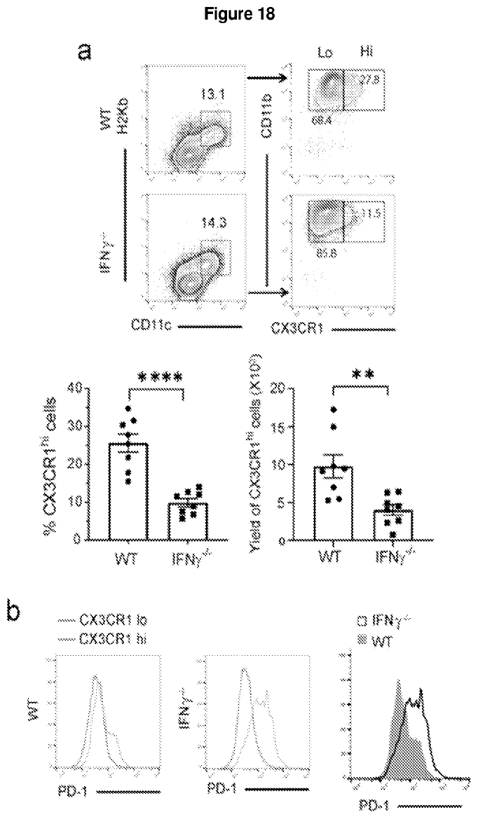

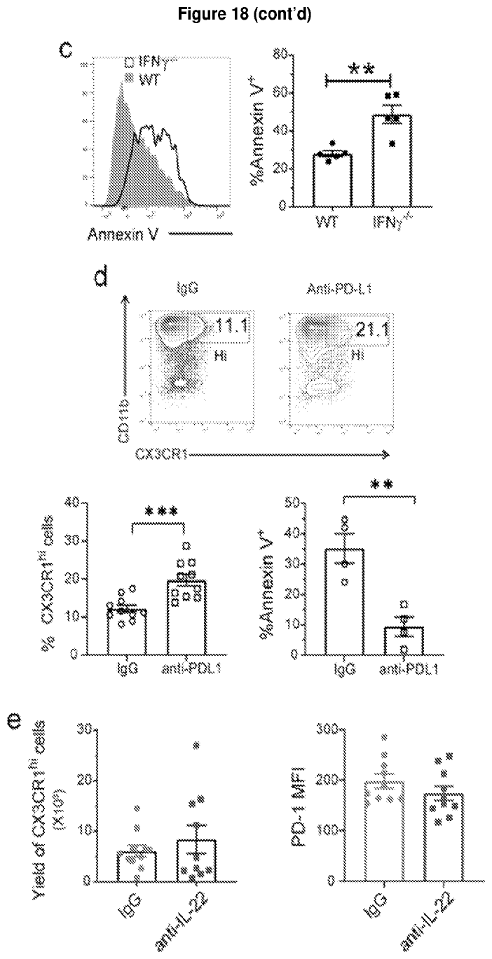

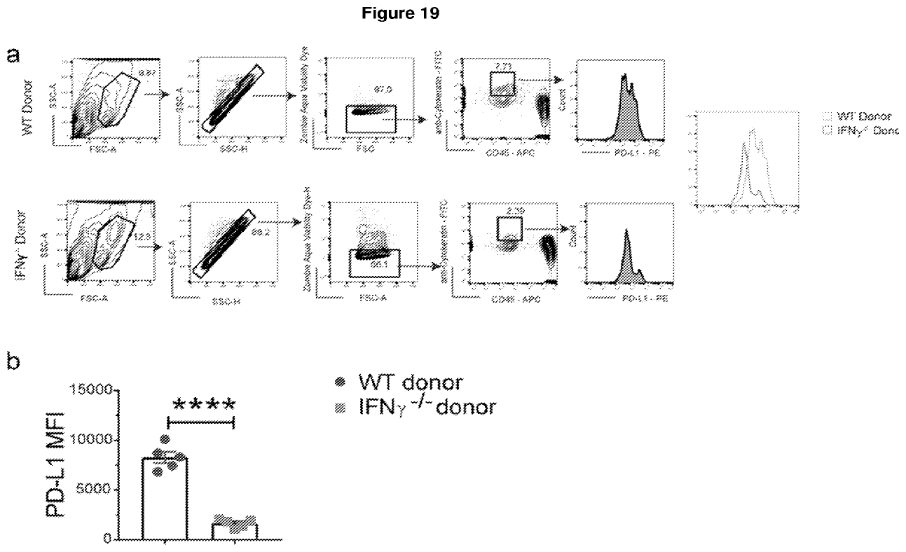

CX3CR1^{hi} MNP cells in the gut tissue have stronger ability to down-regulate inflammatory responses and prevent bacterial translocation as compared to CX3CR1^{lo} MNP cells^{19,51}. Therefore, whether there were any abnormal changes in CX3CR1^{hi} MNP during IFN-γ^{-/-}CD8⁺T cell-mediated gut-aGVHD pathogenesis was explored. By 10 days after HCT, CX3CR1⁺cells were all donor-type, and the CX3CR1⁺cells included CX3CR1^{lo} and CX3CR1^{hi} populations (FIG. 17). The CX3CR1^{hi} MNP in the colon tissues of recipients of WT CD8⁺or IFN-γ^{-/-}CD8⁺T cells expressed higher levels of CD11c, CD11b, F4/80, CD64, MerTK, IL10R, and CSF1-R, as compared with CX3CR1^{lo} MNP (FIG. 17). The percentages and yields of CX3CR1^{hi} MNP in the colon tissue were markedly lower in recipients given IFN-γ^{-/-}CD8⁺T cells than in those given WT CD8⁺T cells (FIG. 18a). The CX3CR1^{hi} MNP in recipients of IFN-γ^{-/-}CD8⁺T cells expressed higher levels of PD-1 (FIG. 18b) and showed higher percentages of apoptotic Annexin V⁺cells (FIG. 18c). Blockade of PD-1 interaction with PD-L1 by anti-PD-L1 mAb significantly increased the percentage of CX3CR1^{hi} MNP in recipients of IFN-γ^{-/-}CD8⁺T cells (FIG. 18d), although intestinal epithelial cells of those recipients expressed lower levels of PD-L1, as compared with recipients of CD8⁺T cells (FIGS. 19a and 19b). Anti-IL-22 had no effect on the yield of CX3CR1^{hi} MNP or their expression of PD-1 in recipients given IFN-γ^{-/-}CD8⁺T cells (FIG. 18e). These results indicate that gut-aGVHD induced by IFN-γ^{-/-}CD8⁺T cells is associated with lower numbers of CX3CR1^{hi} MNP in the colon tissue independent of Tc22 expansion.

### Example 13. Preservation of Donor-Type CX3CR1^{hi} MNP Reverses Gut-aGVHD Induced by IFN-γ^{-/-}CD8⁺T Cells

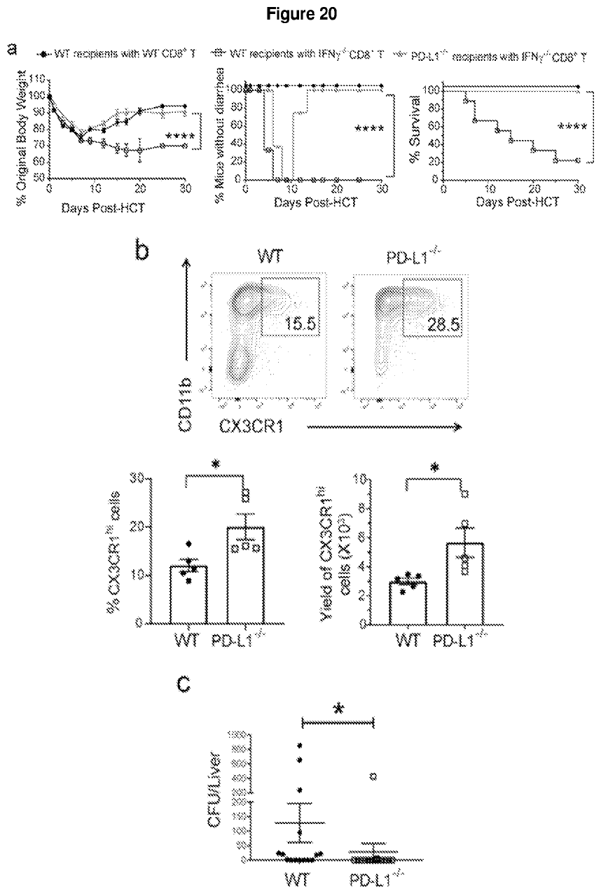

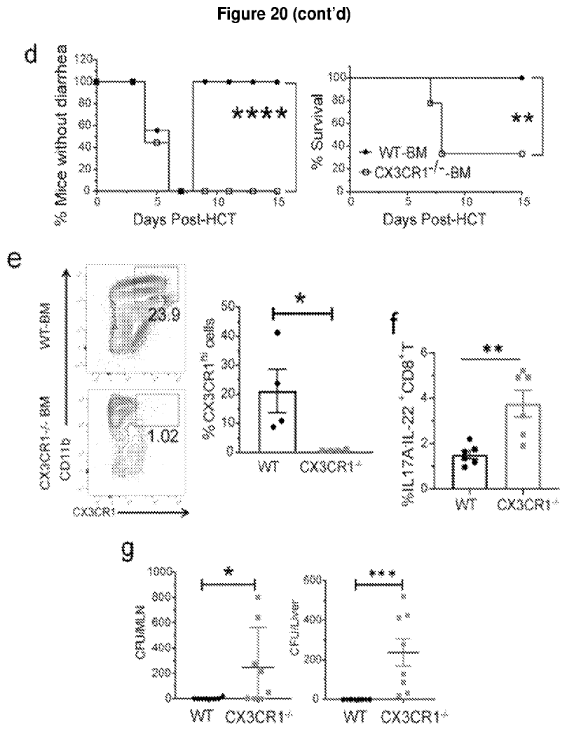

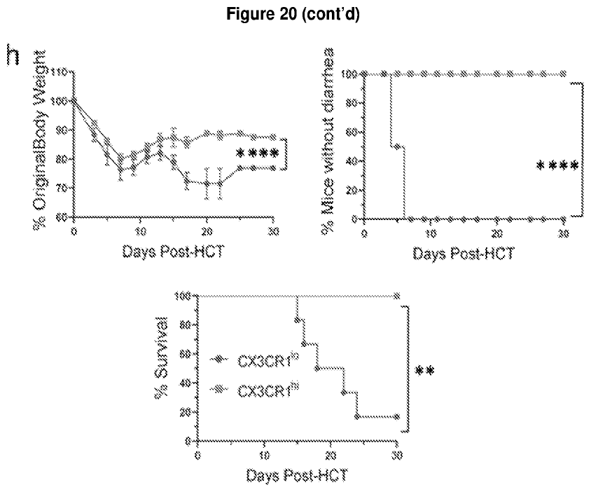

Whether CX3CR1^{hi} MNP could be preserved in PD-L1^{-/-}recipients and whether such preservation could prevent gut-aGVHD were tested. At ˜7 days after HCT, diarrhea developed in both WT and PD-L1^{-/-}recipients of IFN-γ^{-/-}CD8⁺T cells, but not in WT recipients of WT CD8⁺T cells. WT recipients of IFN-γ^{-/-}CD8⁺T cells continued to have diarrhea, and most of them died by 30 days after HCT, but PD-L1^{-/-}recipients of IFN-γ^{-/-}CD8⁺T cells gradually recovered and became diarrhea-free, and all survived for more than 30 days after HCT (FIG. 20a). Recovery in PD-L1^{-/-}recipients was associated with higher percentages and yields of CX3CR1^{hi} MNP in colon tissue (FIG. 20b) and lower levels bacterial translocation into the liver (FIG. 20c).

To validate the protective role of CX3CR1^{hi} MNP in the gut-aGVHD recipients, TCD-BM cells from WT or CX3CR1^{-/-}donors that could not generate CX3CR1^{hi} MNP cells were transplanted together with IFN-γ^{-/-}CD8⁺T cells into PD-L1^{-/-}recipients. Recipients given WT-donor TCD-BM cells developed diarrhea about 5 days after HCT but spontaneously recovered by 7 days after HCT, and all survived for more than 15 days after HCT. In contrast, recipients given CX3CR1^{-/-}donor BM cells developed diarrhea without subsequent recovery, and 67% (6/9) of the recipients died within 10 days after HCT (FIG. 20d). The CX3CR1^{-/-}-BM cells did not restore CX3CR1^{hi} MNP (FIG. 20e). The lack of CX3CR1^{hi} MNP led to greater expansion of Tc22 cells and higher bacterial translocation into the MLN and liver tissues after HCT (FIGS. 20f and 20g).

In further experiments, CX3CR1^{hi} or CX3CR1^{lo} MNP (0.5×10⁶) from PD-L1^{-/-}recipients of IFN-γ^{-/-}CD8⁺T cells at day 10 after HCT was transferred into WT recipients of IFN-γ^{-/-}CD8⁺T cells at day 1 after HCT. Recipients given CX3CR1^{lo} MNP developed diarrhea and lost body weight, and most died within 30 days after HCT. Recipients given CX3CR1^{hi} MNP did not develop diarrhea and had less weight loss, all survived for more than 30 days (FIG. 20h). Therefore, depletion of protective donor-type CX3CR1^{hi} MNP cells is also required for induction of gut-aGVHD by IFN-γ^{-/-}CD8⁺T cells.

### Example 14. Depletion of CX3CR1^{hi} MNP Contributes to Bacterial Translocation in SR-gut-aGVHD

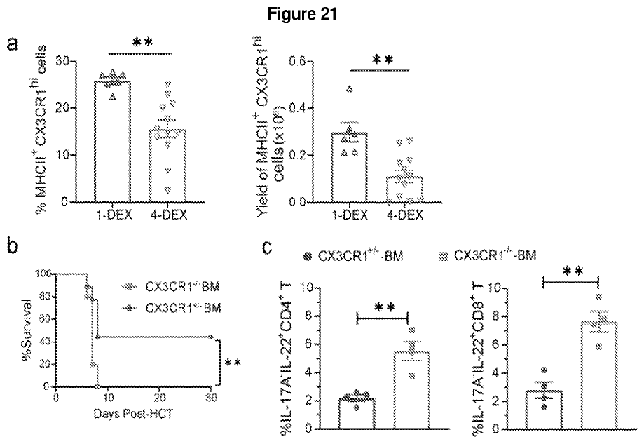

Since DEX-treatment reduced IFN-γ-producing Th1/Tc1 (FIG. 1), and 4-DEX-treatment significantly increased bacterial translocation as compared with 1-DEX-treament (FIG. 4e), the impact of 4-DEX treatment on donor-type CX3CR1^{hi} MNP in the colon was tested. At 25 days after HCT, the percentages and yields of donor-type CX3CR1^{hi} MNP cells in the colon tissues were lower in recipients given 4-DEX treatment than in those given 1-DEX treatment (FIG. 21a). Comparing recipients after 1-DEX-treatment, gut-aGVHD was more severe in recipients given CX3CR1^{-/-}BM than in recipients given CX3CR1^{+/-}BM (FIG. 21b). The exacerbation of gut-aGVHD in recipients given CX3CR1^{-/-}BM was associated with expansion of IL-17A⁻IL-22⁺CD4⁺and CD8⁺Th/Tc22 cells on day 7 after HCT (FIG. 21c). These results suggest that depletion of donor-type CX3CR1^{hi} MNP cells contributes to SR-gut-aGVHD pathogenesis and that in the absence of CX3CR1^{hi} MNP cells, 1-DEX treatment augments expansion of donor-type Th/Tc22 cells.

### Example 15. Colon Tissue of a Patient with Steroid-Refractory Gut GVHD is Characterized with Severe Infiltration of IL-22-Producing T Cells

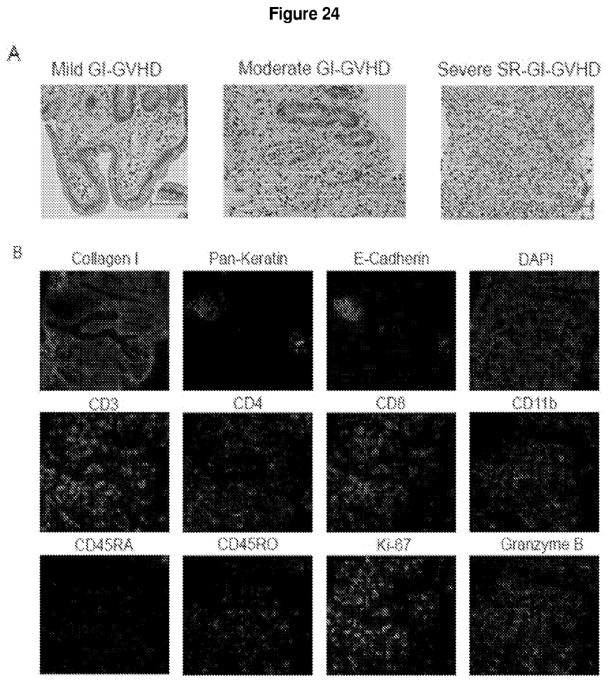

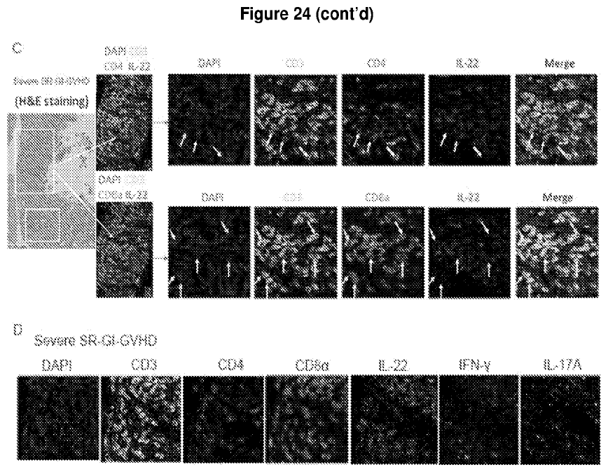

The colon tissue of murine recipients with steroid refractory gut GVHD (SR-Gut-GVHD) is characterized with infiltration of IL-22-producing CD4⁺and CD8⁺T cells. To test whether it was the case in the colon tissue of patients with SR-Gut-GVHD), colon biopsy tissue slides were obtained from three patients who had mild-Gut-GVHD, moderate-Gut-GVHD, and severe SR-Gut-GVHD, respectively, as determined by H&E staining histopathology (FIG. 24A).

To further assess the spatial distribution of colon tissue infiltrating immune cells including CD4⁺, CD8⁺T cells and myeloid cells in situ, a panel of 15 antibodies (Table 1) was combined with a DNA staining to perform imaging mass cytometry (IMC).

TABLE 1
Imaging-Mass Cytometry Antibody Panel

| | | | | | Product | Final |
|---|---|---|---|---|---|---|
| | Tag | Antigen | Clone | Supplier | ID | dilution |
| 1 | 148Nd | Pan-Keratin | C11 | Fluidigm | 3148020D | 1:100 |
| 2 | 149Sm | CD11b | EPR1344 | Fluidigm | 3149028D | 1:50 |
| 3 | 156Gd | CD4 | EPR6855 | Fluidigm | 3156033D | 1:50 |
| 4 | 158Gd | E-Cadherin | 24E10 | Fluidigm | 3158029D | 1:100 |
| 5 | 162Dy | CD8a | C8/144B | Fluidigm | 3162034D | 1:50 |
| 6 | 166Er | CD45RA | HI100 | Fluidigm | 3166031D | 1:100 |
| 7 | 167Er | Granzyme B | EPR20129- | Fluidigm | 3167021D | 1:100 |
| | | | 217 | | | |
| 8 | 168Er | Ki-67 | B56 | Fluidigm | 3168022D | 1:50 |
| 9 | 169Tm | Collagen | Polyclonal | Fluidigm | 3169023D | 1:100 |
| | | Type I | | | | |
| 10 | 170Er | CD3 | Polyclonal | Fluidigm | 3170019D | 1:50 |
| 11 | 173Yb | CD45RO | UCHL1 | Fluidigm | 3173016D | 1:100 |
| 12 | 191 Ir/193Ir | DAPI | NA | Fluidigm | 201192A | 1:200 |
| 13(Self | 152Sm | IL-22 | Polyclonal | R&D | AF782, | 1:25 |
| conjugated) | | | | systems, | 201152A | |
| | | | | Fluidigm | | |
| 14(Self | 153Eu | IFN-γ | Polyclonal | R&D | AF-285-NA, | 1:25 |
| conjugated | | | | systems, | 201153A | |
| | | | | Fluidigm | | |
| 15(Self | 163Dy | IL-17A | Polyclonal | R&D | AF-317-NA, | 1:50 |
| conjugated | | | | systems, | 201163A | |
| | | | | Fluidigm | | |

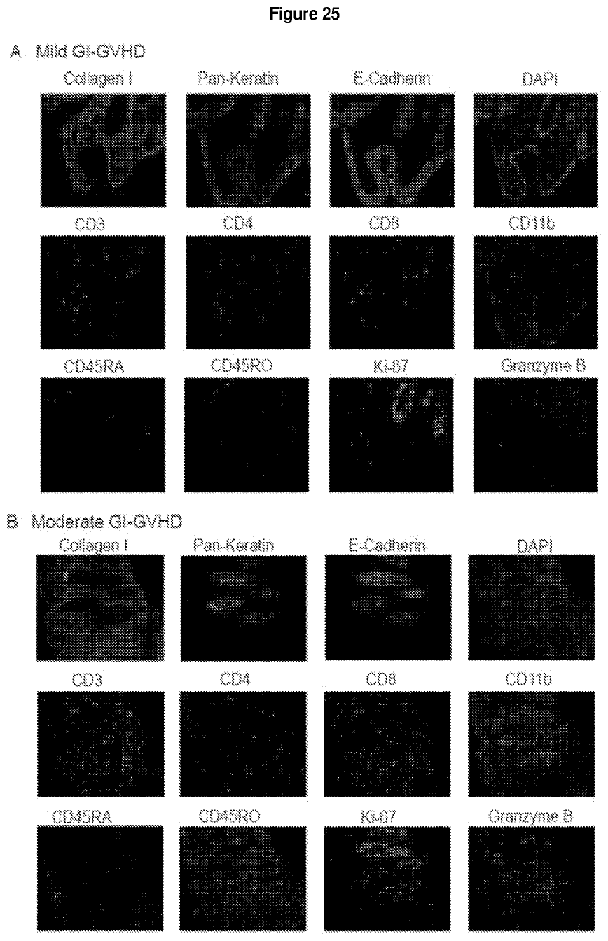

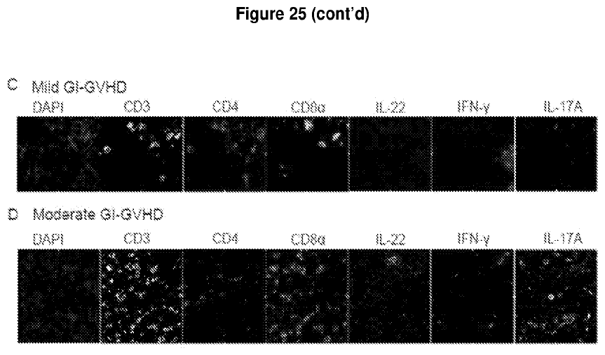

Collagen I staining was used to visualize the extracellular matrix of the basement membrane; the epithelium and lamina propria were distinguished as Pan-Keratin⁺E-cadherin⁺and Pan-Keratin⁻E-cadherin³¹ , respectively (FIGS. 24B, 25A and 25B). Intestinal epithelia damage in the severe SR-Gut-GVHD colon tissue is obvious, as indicated by that few Pan-Keratin⁺E-cadherin⁺cells were visualized, while many Pan-Keratin⁺E-cadherin⁺cells were found in the mild and moderate GI-GVHD (FIGS. 24B, 25A and 25B). The disease severity was also associated with the level of T cell infiltration, much more T cells, especially CD8⁺T cells, were identified in the severe SR-Gut-GVHD tissue sample, as compared to mild Gut-GVHD and moderate-Gut-GVHD; most CD4⁺and CD8⁺T cells colocalization with CD11b⁺myeloid cells in the lamina propria (FIGS. 24B, 25A and 25B). Differential expression of CD45RA and CD45RO allowed for further discrimination of CD45RA+CD45RO⁻naïve T (TN) cells from CD45RA⁻CD45RO⁺memory T (TM) cells in the lamina propria, majority of the CD3⁺CD4⁺or CD8⁺T cells were TM cells regardless of GVHD severity (FIGS. 24B, 25A and 25B). In the severe SR-Gut-GVHD, most of T cells are Ki-67⁺and Granzyme B⁺, indicating their proliferation capacity and cytotoxicity function. In contrast, only small portion of Ki-67⁺and Granzyme B⁺T cells existed in the colon tissues of mild-and moderate-Gut-GVHD (FIGS. 24B, 25A and 25B). In addition, greater numbers of IL-22⁺CD4⁺and CD8⁺T cells were clustered in the tissue of severe SR-Gut-GVHD, while nearly no IL-22⁺CD4⁺or CD8⁺T cells were observed in the tissues of mild- or moderate-Gut-GVHD (FIGS. 24C, 25C and 25D). In addition, those IL-22⁺CD4⁺and CD8⁺T cells in the tissue of severe SR-Gut-GVHD frequently expressed IFN-γ or IL-17A, while those in the tissue of mild or moderate Gut-GVHD were not (FIGS. 24D, 25C and 25D). Collectively, these results indicate that there may be increased infiltration of IL-22⁺TM cells in the colon tissue of SR-Gut-GVHD patient. This is consistent with the observations with murine SR-Gut-GVHD models.

### Example 16. Ceacam-1 Expression is Upregulated on the Colon Epithelia Cells of the Mice with SR-Gut-GVHD

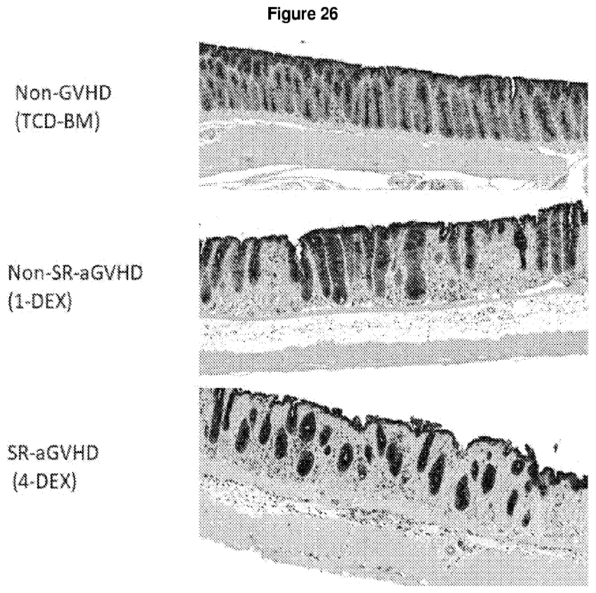

The murine model of SR-Gut-GVHD was used to test the role of ceacam-1 in the pathogenesis of SR-Gut-GVHD. Lethal TBI-conditioned BALB/c mice (H-2^{d}) were given spleen (SPL) cells containing 0.75×10⁶ T cells together with bone marrow (BM) cells (2.5×10⁶) from MHC-mismatched C57BL/6 (H-2^{b}) donors, recipients were given a single injection of dexamethasone (DEX) at 5 mg/kg on day 3 post HSCT (1-DEX) or given additional three injections of DEX on 10, 15 and 20 post HSCT (4-DEX). ceacam-1 expression was evaluated by immunohistochemistry (INC) staining on days 25 post HSCT, compared to non-GVHD mice received T cell depleted bone marrow (TCD-BM) only. The intensity of ceacam-1 expression was similar in non-SR-Gut-GVHD (1-DEX) and TCD-BM mice; however, enhanced ceacam-1 expression was found in the colon epithelia of SR-Gut-GVHD mice (4-DEX) (FIG. 26). These results suggest that SR-Gut-GVHD is associated with increased expression of ceacam-1 on the colon epithelia cells in the murine model.

### Example 17. Host Ceacam-1 Deficiency Ameliorates SR-Gut-GVHD but not Non-SR-GVHD

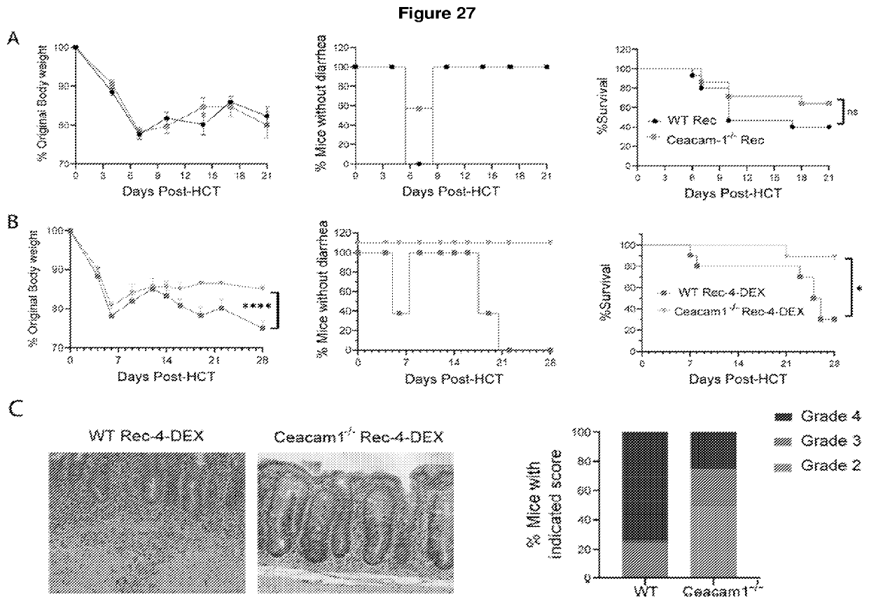

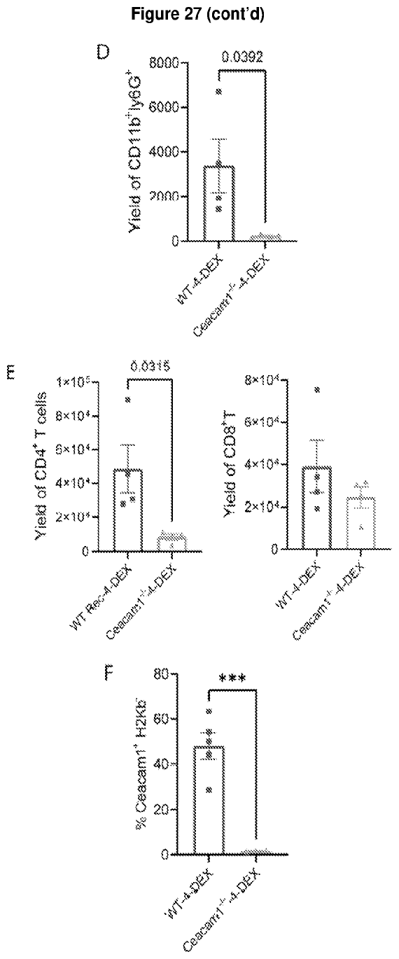

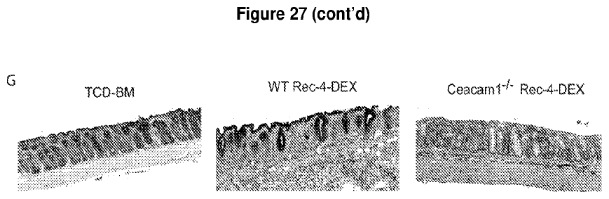

The role of host ceacam-1 in the induction of non-SR-Gut-GVHD was tested. Lethal TBI-conditioned WT or ceacam-1^{-/-}BALB/c mice (H-2^{d}) were given spleen (SPL) cells containing 0.75×10⁶ T cells together with bone marrow (BM) cells (2.5×10⁶) from MHC-mismatched C57BL/6 (H-2^{b}) donors. Compared with WT recipients, ceacam-1^{-/-}recipients developed similar clinical GVHD symptoms, as suggested by little difference in bodyweight loss or diarrhea, and around 40-60% recipients in both groups died within 3 weeks post HSCT (FIG. 27A). These results suggest that ceacam-1 deficiency in host has no significant impact on the induction of non-SR-GVHD.

Since host colon epithelia cell expression of ceacam-1 was upregulated in the SR-Gut-GVHD recipients (FIG. 26), the role of host ceacam-1 in the induction of SR-Gut-GVHD was tested. Briefly, WT or ceacam-1^{-/-}BALB/c mice were given transplants from allogeneic C57BL/6 donor as disclosed above, days 3, 10, 15 and 20 after HCT, recipients were given total four injections of dexamethasone (DEX) at 5 mg/kg. While all of WT recipients showed diarrhea, none of ceacam-1^{-/-}showed diarrhea; as compared to WT recipients, ceacam-1^{-/-}recipients also showed increase of bodyweight and marked increase of survival (90% versus 30%) by day 28 after HCT (FIG. 27B). At the same time, ceacam-1^{-/-}recipients showed reduced colon tissue infiltration and epithelia damage with better gland structure preservation, compared to WT recipients (FIG. 27C). ceacam-1^{-/-}recipients also showed reduction in the numbers of infiltrating donor CD11b⁺ly6G⁺neutrophils as well as donor CD4⁺and CD8⁺T cells in the colon tissues (FIGS. 27D and 27E). The lack of ceacam-1 expression in the colon tissue epithelial cells of ceacam-1^{-/-}recipients was validated by flow cytometry analysis of host-type H-2K^{b-}ceacam-1⁺cells in the colon tissue (FIG. 27F) and IHC staining of ceacam-1 in the colon tissues (FIG. 27G). The infiltrating donor cells also appeared to express high levels of ceacam-1, and ceacam-1 deficiency in the host tissue was associated with reduction of infiltration of ceacam-1⁺donor cells (FIG. 27G). These results indicate that host ceacam-1 deficiency ameliorates SR-Gut-GVHD in association with reduction of donor T cell and neutrophil infiltration in the colon tissues.

### Example 18. Ceacam-1 Deficiency in Host Intestinal Parenchymal Cells but not Host Hematopoietic Cells Ameliorates SR-Gut-GVHD

Since host ceacam-1 deficiency was able to ameliorate SR-Gut-GVHD in association with the reduced infiltration of donor T cells and neutrophils in the colon, whether ceacam-1 deficiency in host parenchyma cells or hematopoietic cells contributed to reduction of SR-Gut-GVHD was tested. To address this question, the bone marrow chimeras with ceacam-1 deficiency only in parenchyma cells including intestinal epithelial cells (IEC-ceacam-1^{-/-}chimera) were established by engrafting myeloablative TBI-conditioned ceacam-1^{-/-}BALB/c mice with TCD-BM (10×10⁶) from WT-BALB/c mice. Conversely, bone marrow chimeras with ceacam-1 deficiency in the hematopoietic cells (HC-ceacam-1^{-/-}-chimeras) were established by engrafting WT BALB/c mice with TCD-BM from ceacam-1^{-/-}BALB/c mice. Control WT-chimeras were established by reconstituting TBI-conditioned BALB/c with TCD-BM cells from WT-BALB/c mice.

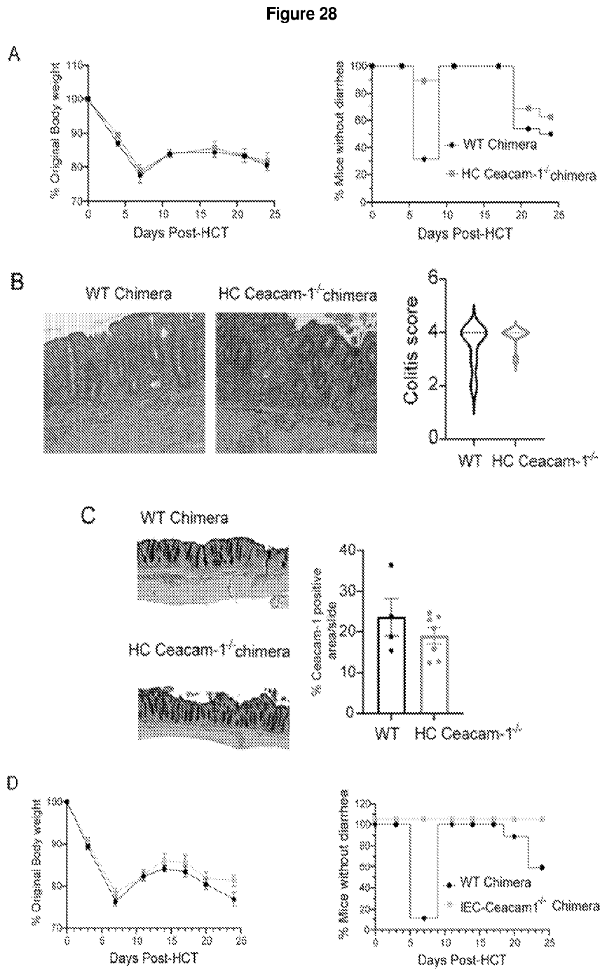

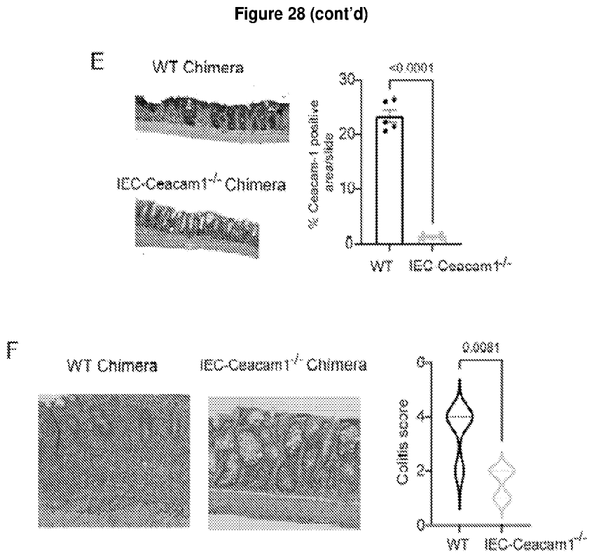

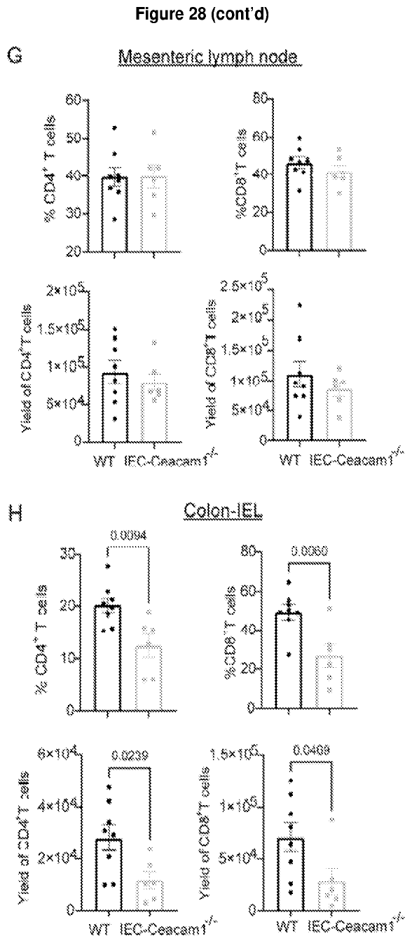

Two months after establishing chimeras, lethal TBI-conditioned WT-chimeras, HC-ceacam-1^{-/-}-chimeras, and IEC-ceacam-1^{-/-}-Chimeras were transplanted with SPL cells together with BM cells from C57BL/6 donors and given 4-DEX treatment to induce SR-Gut-GVHD. First, the WT-Chimeras were compared with HC-ceacam-1^{-/-}-Chimeras and no difference in bodyweight loss, diarrhea, histopathology or host colon tissue expression of ceacam-1 was observed as measured with IHC (FIGS. 28A-28C). These results indicate that ceacam-1 deficiency in host hematopoietic cells does not have significant impact on SR-Gut-GVHD pathogenesis.

Second, the WT-Chimeras were compared with IEC-ceacam-1^{-/-}-Chimeras. There was no difference in bodyweight loss between the IEC-ceacam-1^{-/-}Chimeras and WT-Chimeras; however, while nearly all WT-Chimeras developed diarrhea, IEC-ceacam-1^{-/-}chimeras were totally diarrhea-free (FIG. 28D). At day 25 after HSCT, the lack of ceacam-1 expression by host IEC in IEC-ceacam-1^{-/-}chimeras was validated by HIC staining of the colon tissues (FIG. 28E). There was also a marked reduction of ceacam-1⁺donor cells in the colon tissues as compared with that of WT-Chimeras (FIG. 28E) and marked reduction of infiltration and epithelial tissue damage as compared with that of WT-chimeras (FIG. 28F). Furthermore, in line with the clinical GVHD manifestation and colon pathology, the percentage and yield of both donor CD4⁺and CD8⁺T cells were significantly reduced in the colon-IEL of IEC-ceacam-1^{-/-}chimeras as compared with that of WT-chimeras, although no difference in the MLN (FIGS. 28G and 28H). These results indicate that ceacam-1 deficiency on host parenchymal intestinal epithelial cells ameliorates SR-Gut-GVHD.

### Example 19. Ceacam-1 Deficiency in Host Intestinal Epithelial Cells Results in Trans-Differentiation of Pathogenic Th/Tc22 Cells into Tregs Cells and Reduction of Th/Tc1 Cells in MLN

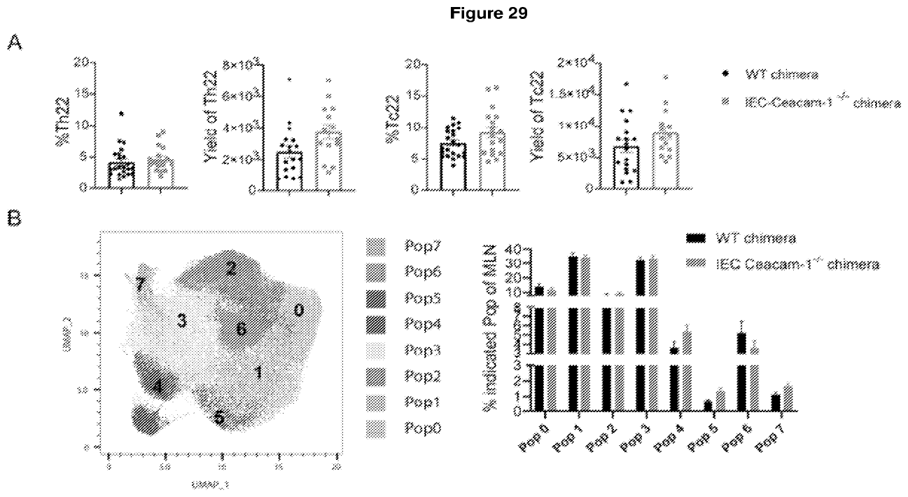

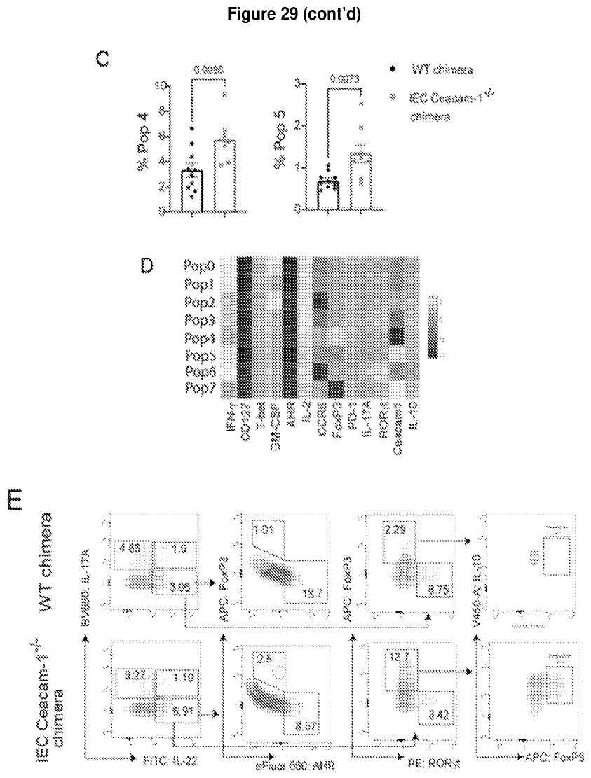

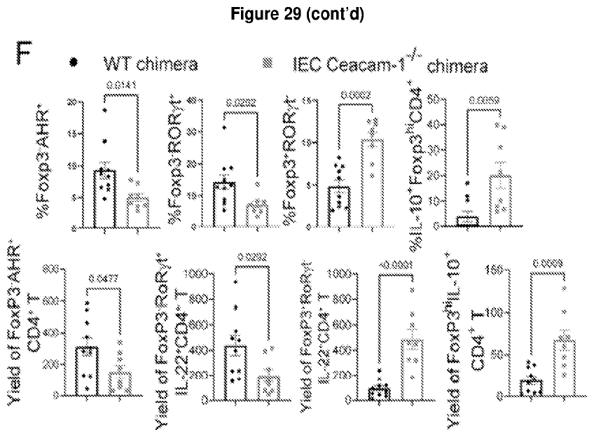

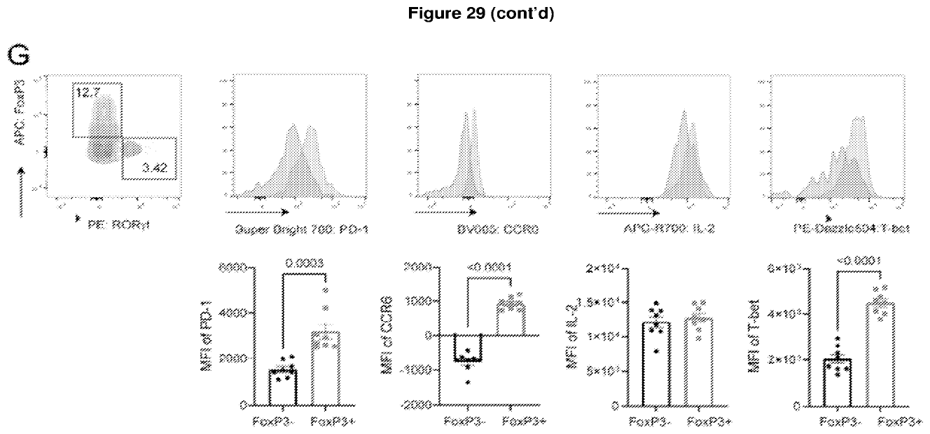

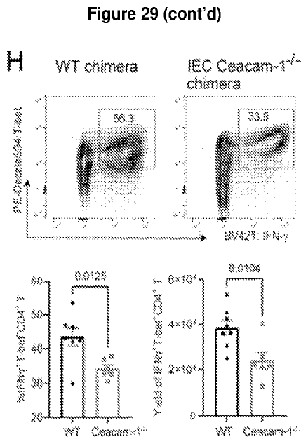

Whether ceacam-1 deficiency on host intestinal epithelia cells impacts on the differentiation and expansion of Th/Tc22 cells was tested. At days 25 post HSCT, the percentage and yield of Th/Tc22 subsets in the MLN were analyzed. Surprisingly, there was no difference in the percentage and yield of donor Th22 (IL-22⁺IL17A⁻CD4⁺T) and Tc22 (IL-22⁺IL17A⁻CD8⁺T) cells in the MLN of WT-Chimeras and IEC-ceacam-1^{-/-}-chimeras (FIG. 29A). Next, whether the functional markers of Th/Tc22 cells from IEC-ceacam-1^{-/-}-Chimeras was distinguishable from those in WT-Chimeras was tested with full spectrum flow cytometry analysis. The donor Th22 cells from MLN of both WT-Chimeras and IEC-ceacam-1^{-/-}-Chimeras were grouped into 8 distinct clusters (FIG. 29B), cluster 4 and 5 are significantly expanded in the MLN of host IEC-ceacam-1^{-/-}-Chimera as compared to WT-chimeras (FIG. 29C). Furthermore, combinational analysis of multiple surface markers, cytokines and transcriptional factors, including CD127, CCR6, PD-1, ceacam-1, IL-2, IFN-γ, GM-CSF, IL17A, IL-10, T-bet, RORγt, AHR, and FoxP3 were applied to further evaluate the signature of individual clusters, especially cluster 4 and 5. The Heatmap plot (FIG. 29D) reflected that Cluster 4 was of high expression of Foxp3 as compared to other clusters, while cluster 5 was of upregulated ceacam-1 expression.

The functional markers of Treg cells in the cluster 4 were analyzed. CD4⁺T cells from the MLN of WT-Chimeras and IEC-ceacam-1^{-/-}-Chimeras are first shown in IL-17A versus IL-22, and the gated IL-22⁺IL-17A⁻cells are shown in AHR versus FoxP3 or RORγt versus FoxP3; the gated Foxp3⁺cells are shown in FoxP3 versus IL-10 again (FIG. 29E). In agreement with what was found in the high dimension plot analysis (FIGS. 29C-29D), the percentage and yield of FoxP3⁺RORγt⁻and FoxP3^{hi}IL-10⁺cells were markedly increased, while the FoxP3⁻RORγt⁺and FoxP3⁻AHR⁺cells were markedly decreased in the IEC-ceacam-1^{-/-}-Chimeras as compared with the WT-Chimeras (FIGS. 29E-29F). The expanded FoxP3⁺Tregs expressed higher level of PD-1, CCR6 and T-bet as compared to non-Tregs (FIG. 29G). In addition, the Th/Tc1 cells in MLN were evaluated. T-bet⁺IFN-γ⁺CD4⁺and CD8⁺T cells were significantly reduced in the IEC-ceacam-1^{-/-}-Chimeras as compared to WT chimeras (FIG. 29H). Overall, these results have established that ceacam-1 deficiency on host intestinal epithelial cells leads to trans-differentiation of pathogenic Th/Tc22 cells into effector-memory Treg cells and inhibition of Th/Tc1 cell expansion in MLN.

### Example 20. Ceacam-1 Deficiency in Intestinal Epithelial Cells Leads to Expansion of Tregs While Reduction of Th/Tc1 and Th/Tc22 Cells in the Colon Tissues

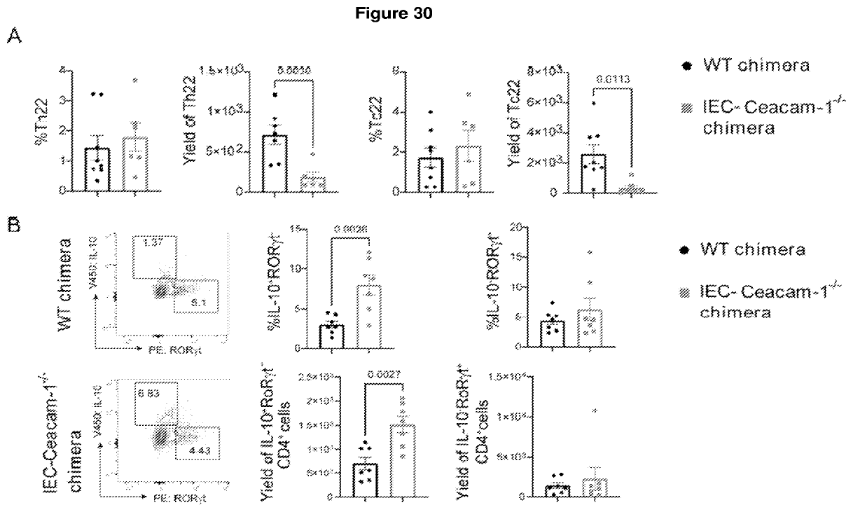

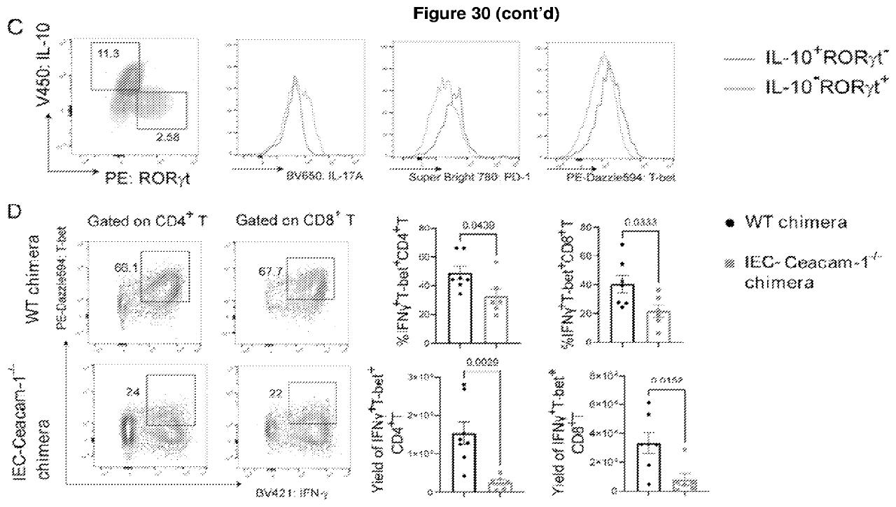

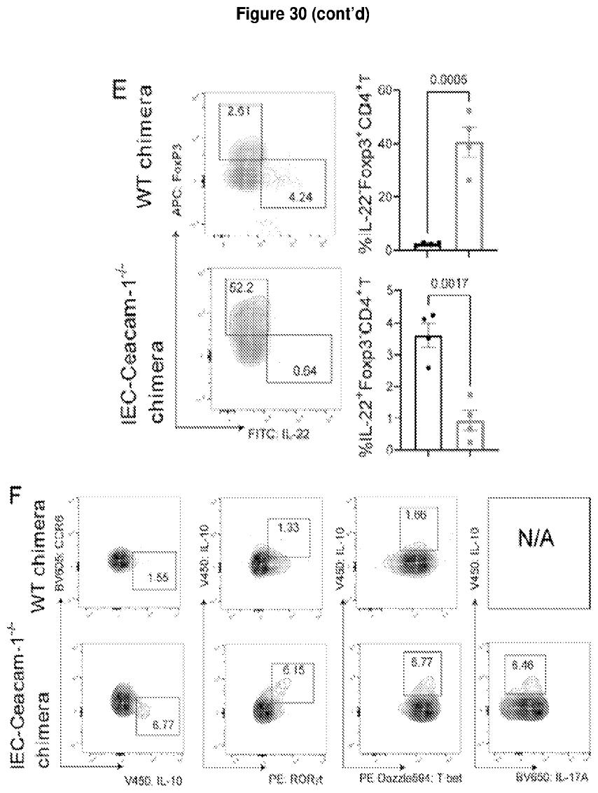

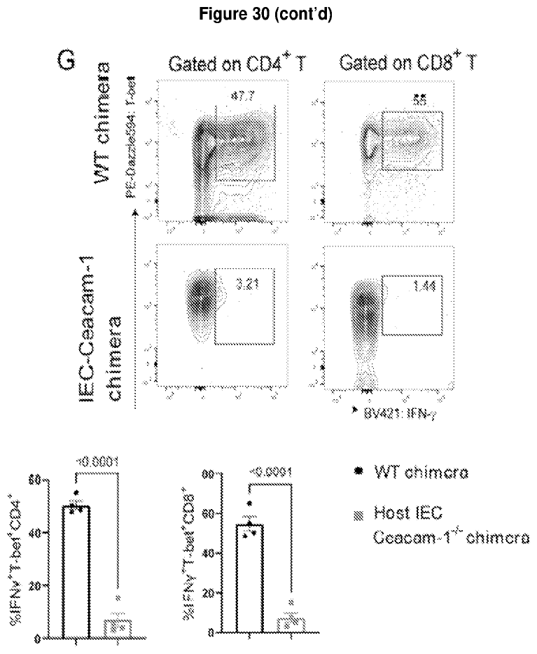

Given the impact of host epithelia ceacam-1 deficiency on the reprograming of Th/Tc22 cells to pTregs in the MLN (FIG. 29), the Th and Tc subsets in the target organ of SR-Gut-GVHD, colon, were analyzed. On day 25 post HSCT, although the percentage of Th/Tc22 cells had no difference, the yield of donor Th/Tc22 cells was markedly reduced in the colon intraepithelial compartment of IEC-ceacam-1^{-/-}-Chimeras, as compared with WT-Chimeras (FIG. 30A). In addition, the percentage and yield of IL-10⁺RORγt⁻CD4⁺T cells were significantly increased in the host IEC-ceacam-1^{-/-}- Chimeras, compared to WT-chimeras, while no difference in the percentage and yield of IL-10⁻RORγt⁺CD4⁺T cells was observed (FIG. 30B). The IL-17A, PD-1 and T-bet expression by IL-10⁺RORγt⁻CD4⁺T cells was compared with IL-10⁻RORγt⁺ CD4⁺T cells. Notably, PD-1 and T-bet expression was upregulated while IL-17A expression was downregulated in the IL-10⁺RORγt⁻CD4⁺T cells when compared with IL-10⁻RORγt⁺ CD4⁺T cells (FIG. 30C). In line with that donor Th/Tc1cells in the MLN were significantly reduced in the IEC-ceacam-1^{-/-}-Chimeras (FIG. 29), there was also a significantly reduction of T-bet⁺IFN-γ⁺CD4⁺and CD8⁺T cells in the colon epithelial tissue of IEC-ceacam-1^{-/-}-Chimeras (FIG. 30D).

Furthermore, the impact of host IEC ceacam-1 deficiency on the infiltrating T cells in the colon laminar propria in addition to those in the epithelial tissues, comparing the Th22, Tregs and Th/Tc1 cells, was investigated. Different from the MLN and colon intraepithelial compartment (FIGS. 29, and 30A-30D), there was a dramatic increase in the percentage of IL-22⁻FoxP3⁺Tregs and dramatic reduction in the percentage of Th22 and Th/Tc1 cells in the colon laminar propria compartment (FIGS. 30E-30G). Taken together, ceacam-1 deficiency on host intestinal epithelial cells leads to trans-differentiation of Th/Tc22 cells into peripheral pTreg cells in the MLN and expansion of Tregs while inhibition of Th/Tc1 and Th/Tc22 cells in colon tissues, resulting in amelioration of SR-Gut-GVHD.

### Example 21. Ceacam-1 Deficiency in the Host Intestinal Epithelial Cells Reversed Dysbiosis and Inhibited E. Coli and Ceacam-1⁺IEC Interaction

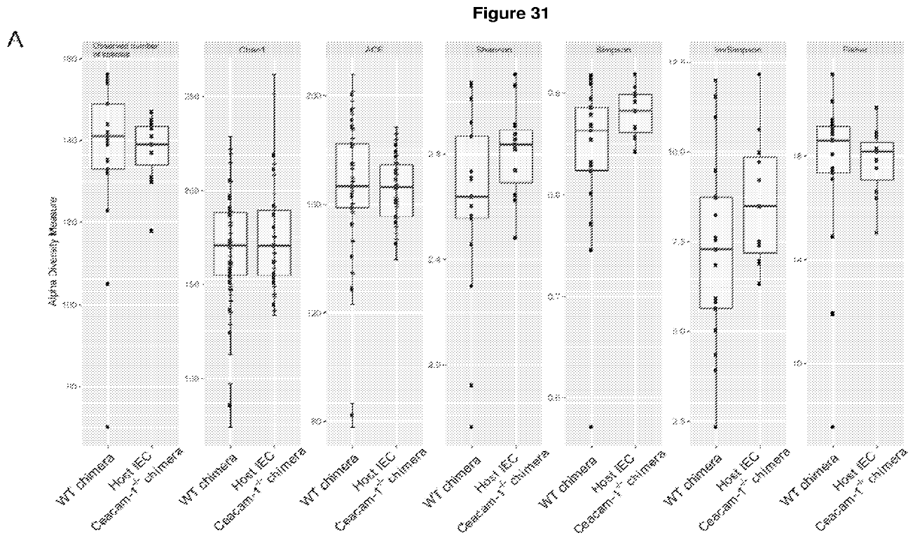

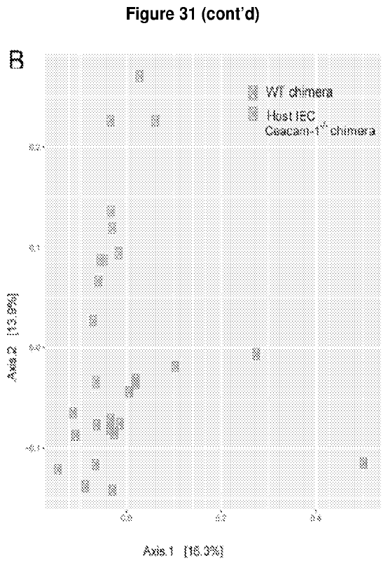

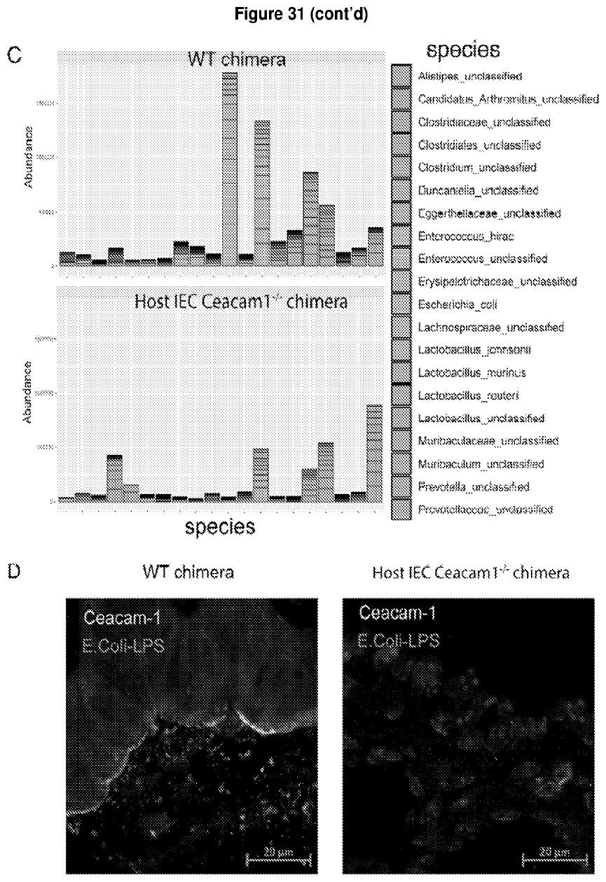

To test whether ceacam-1 deficiency on the host IEC could prevent the dysbiosis in SR-Gut-GVHD, on day 25 after HCT, feces from the ileum of WT-Chimeras or IEC-ceacam-1^{-/-}-Chimeras were analyzed for 16S ribosomal RNA sequences. As compared with WT-Chimeras, IEC-ceacam-1^{-/-}-Chimers showed no difference in bacterial diversity, as judged by the numbers of species, Chao1, ACE, Shannon, simpson, InvSimpson and Fisher index (FIG. 31A). However, principal coordinate analysis (PCA) showed that bacteria from the IEC-ceacam-1^{-/-}-Chimeras were distinguishable from those of WT-Chimeras (FIG. 31B).

Further analyzing bacterial subpopulations at species level showed that IEC-ceacam-1^{-/-}-Chimeras had marked expansion of Clostridales unclassfied and Prevotellaceae_unclassfied and dramatic reduction of E. coli (FIG. 31C). To further visualize the E. Coli interaction with ceacam-1⁺IECs, immunofluorescent staining (IF) for ceacam-1 and E. Coli were performed in the colon tissue of WT-Chimeras and IEC-ceacam-1^{-/-}-Chimeras. The signal of E. Coli-LPS was heavily accumulated near the ceacam-1⁺IECs of the WT-Chimeras recipients but was almost absent near the ceacam-1^{-/-}IEC of IEC-ceacam-1^{-/-}-Chimeras (FIG. 31D). These results indicate that ceacam-1 deficiency in the host intestinal epithelia cells reverses dysbiosis and inhibits E. Coli interaction with and ceacam-1⁺intestinal epithelial cells.

### Example 22. Combination of In Vivo Anti-Ceacam-1 and Anti-Gr-1 Therapy Ameliorates SR-Gut-GVHD

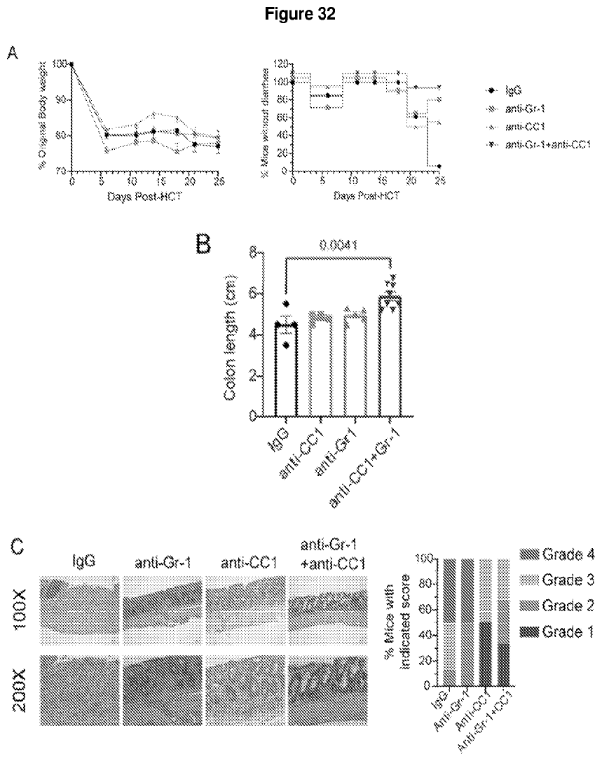

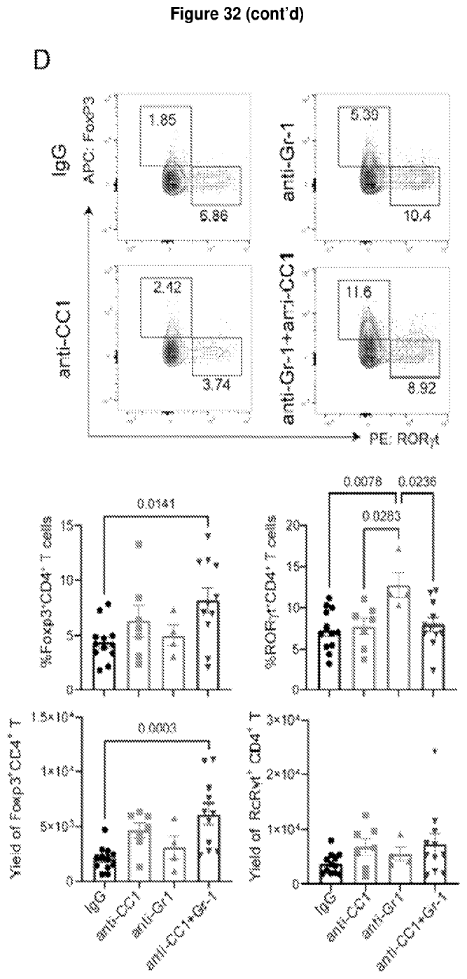

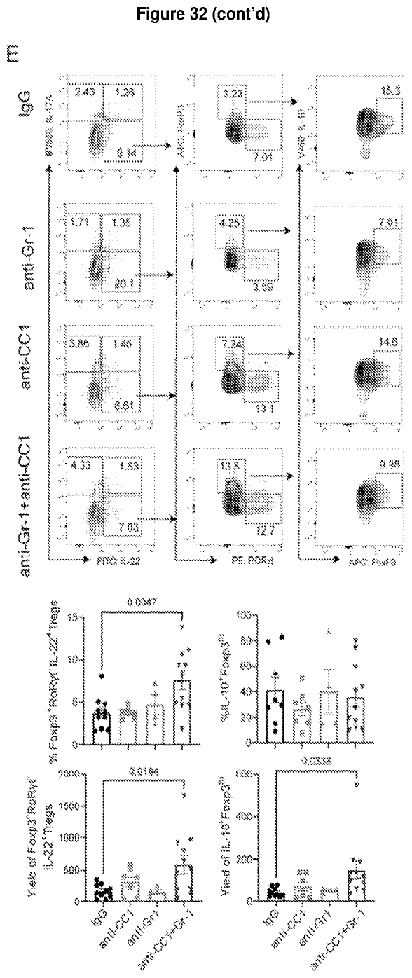

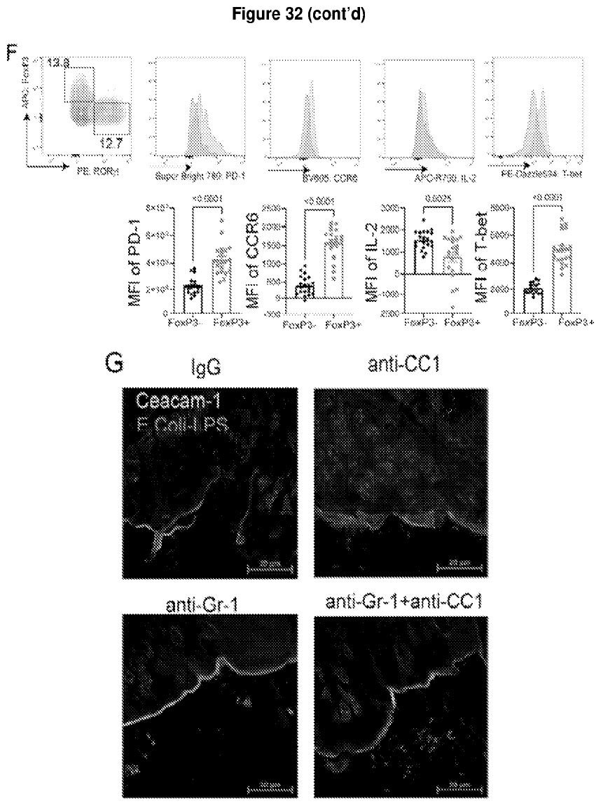

Since host IEC ceacam-1 deficiency was sufficient to ameliorate SR-Gut-GVHD (FIGS. 28-31), whether in vivo administration of anti-ceacam-1 (anti-CC1) mAbs could mimic ceacam-1 deficiency on host IEC was tested. Lethal TBI-conditioned BALB/c mice were given SPL cells together with BM cells from C57BL/6 donors and induced to develop SR-Gut-GVHD by 4 injections of DEX as disclosed above. Started from day 12 after HCT, recipients were given i.p. injections of anti-CC1 mAb or mouse IgG control (100 ug/injection), every three days until day 24. Anti-CC1 treatment did not significantly improve body weight, although reduced mice with diarrhea as compared with IgG treated mice (FIG. 32A).

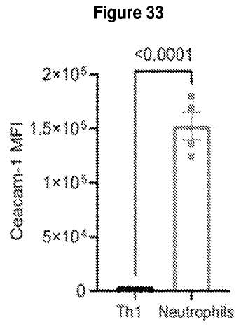

Because neutrophils express high level of ceacam-1 (FIG. 33), and SR-Gut-GVHD is associated with increased neutrophil infiltration in the colon, whether removal of neutrophils could enhance the effect of anti-CC1 treatment was tested. The recipients were given an additional single i.p. injection of anti-Gr-1 mAb (500 ug/mouse) on day 11 post HSCT, with subsequential treatment of anti-CC1 mAb as disclosed above. Recipients given single injection of anti-Gr-1 alone was used as additional control. Intriguingly, combination of anti-Gr-1 with anti-CC1 mAb appeared to further reduce mice with diarrhea (FIG. 32A). Moreover, the colon length is significantly longer than the other three groups (FIG. 32B). Day 25 histopathology showed that as compared with recipients treated with IgG or anti-Gr-1 alone that have mainly grade 3 and 4 damages, anti-CC1 alone totally removed grad 4 damage with remaining grade 1 and 3 damages. Combination treatment of anti-CC1 and anti-Gr-1 further reduced grade 3 damage as compared with anti-CC1 treatment alone (FIG. 32C). Thus, administration of anti-CC1, especially anti-CC1 plus anti-Gr1, ameliorate SR-Gut-GVHD.

On day 25 after HSCT, the T cell subsets were also analyzed by flow cytometry in the MLN. Interestingly, significant expansion in percentage and yield of Foxp3⁺CD4⁺Tregs was found in the mice treated with anti-CC1, especially anti-CC1 and anti-Gr-1 mAb together, while no impact on the RORγt⁺CD4⁺T cells (FIG. 32D). Importantly, two-fold increase of FoxP3⁺population within Th22 cells in the combination treated group compared to other groups was observed (FIG. 32E). The expanded FoxP3⁺Tregs expressed higher level of PD-1, CCR6 and T-bet as compared to RORγt⁺ non-Tregs, although no difference in IL-2 expression was observed (FIG. 32F). Additionally, anti-CC1 and anti-Gr-1 mAb combination treatment appeared to reduce E. Coli colonization on the ceacam-1⁺colon epithelia cells (FIG. 32G).

As demonstrated in the working examples, expansion of Th/Tc22 cells in the colon tissues is in association with SR-Gut-GVHD in patients. With murine models, the intestinal epithelial cells of SR-Gut-GVHD recipients upregulated expression of ceacam-1, and ceacam-1 deficiency in the intestinal epithelial cells effectively prevented SR-Gut-GVHD in association with enhanced trans-differentiation of Th/Tc22 into FoxP3⁺Treg cells in the MLN as well as increase of Treg cells and decrease of Th/Tc1 and Th/Tc22 cells in the colon tissues. The ceacam-1 deficiency in the intestinal epithelial cells also blocked interaction between bacteria and intestinal epithelial cells and reduced bacterial colony formation at the epithelial cell area and reverses dysbiosis. Administration of anti-ceacam-1 in combination of depletion of neutrophils that express high levels of ceacam-1 effectively prevented and reversed SR-Gut-GVHD, although treating acute GVHD with anti-Gr-1 to deplete neutrophils is not practical for acute GVHD patients due to concerns about increase of infection. These results indicate that blockade of homophilic ceacam-1 interactions between bacteria and intestinal epithelial cells via oral administration of ceacam-1 antagonist may be an effective approach for preventing and treating SR-Gut-GVHD and even general Gut-GVHD.

### References

1. Magenau J, Reddy P. Next generation treatment of acute graft-versus-host disease. Leukemia 28, 2283-2291 (2014).
2. Koyama M, Hill G R. The primacy of gastrointestinal tract antigen-presenting cells in lethal graft-versus-host disease. Blood 134, 2139-2148 (2019).
3. Naymagon S, et al. Acute graft-versus-host disease of the gut: considerations for the gastroenterologist. Nat Rev Gastroenterol Hepatol 14, 711-726 (2017).
4. Cash H L, Whitham C V, Behrendt C L, Hooper L V. Symbiotic bacteria direct expression of an intestinal bactericidal lectin. Science 313, 1126-1130 (2006).
5. Vaishnava S, et al. The antibacterial lectin RegIIIgamma promotes the spatial segregation of microbiota and host in the intestine. Science 334, 255-258 (2011).
6. Jenq R R, et al. Regulation of intestinal inflammation by microbiota following allogeneic bone marrow transplantation. The Journal of experimental medicine 209, 903-911 (2012).
7. Eriguchi Y, et al. Graft-versus-host disease disrupts intestinal microbial ecology by inhibiting Paneth cell production of alpha-defensins. Blood 120, 223-231 (2012).
8. Lindemans C A, et al. Interleukin-22 promotes intestinal-stem-cell-mediated epithelial regeneration. Nature 528, 560-564 (2015).
9. Legoff J, et al. The eukaryotic gut virome in hematopoietic stem cell transplantation: new clues in enteric graft-versus-host disease. Nature medicine 23, 1080-1085 (2017).
10. Mathewson N D, et al. Gut microbiome-derived metabolites modulate intestinal epithelial cell damage and mitigate graft-versus-host disease. Nature immunology 17, 505-513 (2016).
11. Shono Y, et al. Increased GVHD-related mortality with broad-spectrum antibiotic use after allogeneic hematopoietic stem cell transplantation in human patients and mice. Sci Transl Med 8, 339ra371 (2016).
12. Behnsen J, et al. The cytokine IL-22 promotes pathogen colonization by suppressing related commensal bacteria. Immunity 40, 262-273 (2014).
13. Ouyang W, O'Garra A. IL-10 Family Cytokines IL-10 and IL-22: from Basic Science to Clinical Translation. Immunity 50, 871-891 (2019).
14. Witte E, Witte K, Warszawska K, Sabat R, Wolk K. Interleukin-22: a cytokine produced by T, NK and NKT cell subsets, with importance in the innate immune defense and tissue protection. Cytokine & growth factor reviews 21, 365-379 (2010).
15. Ivanov, I I, et al. The orphan nuclear receptor RORgammat directs the differentiation program of proinflammatory IL-17+T helper cells. Cell 126, 1121-1133 (2006).
16. Yang X O, et al. T helper 17 lineage differentiation is programmed by orphan nuclear receptors ROR alpha and ROR gamma. Immunity 28, 29-39 (2008).
17. Plank M W, et al. Th22 Cells Form a Distinct Th Lineage from Th17 Cells In Vitro with Unique Transcriptional Properties and Tbet-Dependent Th1 Plasticity. Journal of immunology 198, 2182-2190 (2017).
18. Eyerich S, et al. Th22 cells represent a distinct human T cell subset involved in epidermal immunity and remodeling. The Journal of clinical investigation 119, 3573-3585 (2009).
19. Medina-Contreras O, et al. CX3CR1 regulates intestinal macrophage homeostasis, bacterial translocation, and colitogenic Th17 responses in mice. The Journal of clinical investigation 121, 4787-4795 (2011).
20. Morita N, et al. GPR31-dependent dendrite protrusion of intestinal CX3CR1(+) cells by bacterial metabolites. Nature 566, 110-114 (2019).
21. Niess J H, et al. CX3CR1-mediated dendritic cell access to the intestinal lumen and bacterial clearance. Science 307, 254-258 (2005).
22. Longman R S, et al. CX(3)CR1(+) mononuclear phagocytes support colitis-associated innate lymphoid cell production of IL-22. The Journal of experimental medicine 211, 1571-1583 (2014).
23. Kim M, et al. Critical Role for the Microbiota in CX3CR1(+) Intestinal Mononuclear Phagocyte Regulation of Intestinal T Cell Responses. Immunity 49, 151-163 e155 (2018).
24. Panea C, et al. Intestinal Monocyte-Derived Macrophages Control Commensal-Specific Th17 Responses. Cell reports 12, 1314-1324 (2015).
25. Muller A M, Linderman J A, Florek M, Miklos D, Shizuru J A. Allogeneic T cells impair engraftment and hematopoiesis after stem cell transplantation. Proceedings of the National Academy of Sciences of the United States of America 107, 1 4721-1 4726 (2010).
26. Martin P J, Inamoto Y, Flowers M E, Carpenter P A. Secondary treatment of acute graft-versus-host disease: a critical review. Biology of blood and marrow transplantation: journal of the American Society for Blood and Marrow Transplantation 18, 982-988 (2012).
27. Toubai T, Magenau J. Immunopathology and biology-based treatment of steroid-refractory graft-versus-host disease. Blood 136, 429-440 (2020).
28. Martin P J. How I treat steroid-refractory acute graft-versus-host disease. Blood 135, 1630-1638 (2020).
29. Banuelos J, Lu N Z. A gradient of glucocorticoid sensitivity among helper T cell cytokines. Cytokine & growth factor reviews 31, 27-35 (2016).
30. Betts B C, et al. CD4+T cell STAT3 phosphorylation precedes acute GVHD, and subsequent Th17 tissue invasion correlates with GVHD severity and therapeutic response. Journal of leukocyte biology 97, 807-819 (2015).
31. Toubai T, et al. Murine Models of Steroid Refractory Graft-versus-Host Disease. Scientific reports 8, 12475 (2018).
32. Hanash A M, et al. Interleukin-22 protects intestinal stem cells from immune-mediated tissue damage and regulates sensitivity to graft versus host disease. Immunity 37, 339-350 (2012).
33. Zhao D, et al. Survival signal REG3alpha prevents crypt apoptosis to control acute gastrointestinal graft-versus-host disease. The Journal of clinical investigation 128, 4970-4979 (2018).
34. Lamarthee B, et al. Donor interleukin-22 and host type I interferon signaling pathway participate in intestinal graft-versus-host disease via STAT1 activation and CXCL10. Mucosal immunology 9, 309-321 (2016).
35. Couturier M, et al. IL-22 deficiency in donor T cells attenuates murine acute graft-versus-host disease mortality while sparing the graft-versus-leukemia effect. Leukemia 27, 1527-1537 (2013).
36. Wen F Q, et al. Glucocorticoids modulate TGF-beta production. Inflammation 26, 279-290 (2002).
37. Wine E, et al. Interleukin-6 is associated with steroid resistance and reflects disease activity in severe pediatric ulcerative colitis. Journal of Crohn's & colitis 7, 916-922 (2013).
38. Mantel P Y, Schmidt-Weber C B. Transforming growth factor-beta: recent advances on its role in immune tolerance. Methods in molecular biology 677, 303-338 (2011).
39. Akdis M, Palomares O, van de Veen W, van Splunter M, Akdis CA. TH17 and TH22 cells: a confusion of antimicrobial response with tissue inflammation versus protection. The Journal of allergy and clinical immunology 129, 1438-1449; quiz1450-1431 (2012).
40. Bhaumik S, Basu R. Cellular and Molecular Dynamics of Th17 Differentiation and its Developmental Plasticity in the Intestinal Immune Response. Frontiers in immunology 8, 254 (2017).
41. Ni X, et al. PD-L1 interacts with CD80 to regulate graft-versus-leukemia activity of donor CD8+T cells. The Journal of clinical investigation 127, 1960-1977 (2017).
42. Yang Y G, Qi J, Wang M G, Sykes M. Donor-derived interferon gamma separates graft-versus-leukemia effects and graft-versus-host disease induced by donor CD8 T cells. Blood 99, 4207-4215 (2002).
43. Hulsdunker J, et al. Neutrophils provide cellular communication between ileum and mesenteric lymph nodes at graft-versus-host disease onset. Blood 131, 1858-1869 (2018).
44. Schwab L, et al. Neutrophil granulocytes recruited upon translocation of intestinal bacteria enhance graft-versus-host disease via tissue damage. Nature medicine 20, 648-654 (2014).
45. Tosti N, et al. Infiltration by IL22-Producing T Cells Promotes Neutrophil Recruitment and Predicts Favorable Clinical Outcome in Human Colorectal Cancer. Cancer immunology research, (2020).
46. Zeiser R, Blazar B R. Acute Graft-versus-Host Disease-Biologic Process, Prevention, and Therapy. The New England journal of medicine 377, 2167-2179 (2017).
47. Basu R, et al. Th22 cells are an important source of IL-22 for host protection against enteropathogenic bacteria. Immunity 37, 1061-1075 (2012).
48. Zheng Y, et al. Interleukin-22 mediates early host defense against attaching and effacing bacterial pathogens. Nature medicine 14, 282-289 (2008).
49. Rakoff-Nahoum S, Paglino J, Eslami-Varzaneh F, Edberg S, Medzhitov R. Recognition of commensal microflora by toll-like receptors is required for intestinal homeostasis. Cell 118, 229-241 (2004).
50. Ubeda C, et al. Vancomycin-resistant Enterococcus domination of intestinal microbiota is enabled by antibiotic treatment in mice and precedes bloodstream invasion in humans. The Journal of clinical investigation 120, 4332-4341 (2010).
51. Desalegn G, Pabst O. Inflammation triggers immediate rather than progressive changes in monocyte differentiation in the small intestine. Nature communications 10, 3229 (2019).
52. Yi T, et al. Reciprocal differentiation and tissue-specific pathogenesis of Th1, Th2, and Th17 cells in graft-versus-host disease. Blood 114, 3101-3112 (2009).
53. Yi T, et al. Absence of donor Th17 leads to augmented Th1 differentiation and exacerbated acute graft-versus-host disease. In: Blood) (2008).
54. Gartlan K H, et al. Tc17 cells are a proinflammatory, plastic lineage of pathogenic CD8+T cells that induce GVHD without antileukemic effects. Blood 126, 1609-1620 (2015).
55. Lo B C, et al. IL-22 Preserves Gut Epithelial Integrity and Promotes Disease Remission during Chronic Salmonella Infection. Journal of immunology 202, 956-965 (2019).
56. Doris M. Ponce A A, Ryotaro Nakamura, Karamjeet S. Sandhu,Juliet N. Barker, FRACP, Jinru Shia,Xiaoqiang Yan,William L. Daley, GillianMoore, Samira Fatmi, Cristina Soto, Antonio Gomes, John Slingerland, PaulGiardina, Jonathan U. Peled,Marcel R. M. van den Brink, AlanM. Hanash. A Phase 2 Study of F-652, a Novel Tissue-Targeted Recombinant Human Interleukin-22 (IL-22) Dimer, for Treatment of Newly Diagnosed Acute Gvhd of the Lower GI Tract. In: BBMT) (2020).
57. Takashima S, et al. T cell-derived interferon-gamma programs stem cell death in immune-mediated intestinal damage. Science immunology 4, (2019).
58. Choi J, et al. Baricitinib-induced blockade of interferon gamma receptor and interleukin-6 receptor for the prevention and treatment of graft-versus-host disease. Leukemia (2018).
59. Madsen L, et al. Mice lacking all conventional MHC class II genes. Proceedings of the National Academy of Sciences of the United States of America 96, 10338-10343 (1999).
60. Chakraverty R, et al. An inflammatory checkpoint regulates recruitment of graft-versus-host reactive T cells to peripheral tissues. The Journal of experimental medicine 203, 2021-2031 (2006).
61. Srinagesh H, et al. The MAGIC algorithm probability is a validated response biomarker of treatment of acute graft-versus-host disease. Blood Advances 3(23), 4034-4042 (2019).

1. A method of preventing or treating acute GVHD in a subject receiving a hematopoietic cell transplantation (HCT) or autoimmune colitis comprising administering to the subject an effective amount of an anti-IL-22 antibody, an anti-IL-6 antibody, donor-type CX3CR1^{hi} MNPs, donor-type NK cells, a ceacam-1 antagonist, an anti-Gr-1 antibody or a combination thereof.

2. The method of claim 1, wherein the ceacam-1 antagonist is an anti-ceacam-1 antibody.

3. The method of claim 1 or claim 2, wherein the anti-IL-22 antibody, the anti-IL-6 antibody, the anti-ceacam-1 antibody, or the anti-Gr-1 antibody is a monoclonal antibody.

4. The method of any one of claims 1-3, wherein the anti-IL-22 antibody, the anti-IL-6 antibody, the anti-ceacam-1 antibody, or the anti-Gr-1 antibody is a recombinant antibody.

5. The method of any one of claims 1-4, wherein the anti-IL-22 antibody, the anti-IL-6 antibody, the anti-ceacam-1 antibody, or the anti-Gr-1 antibody is a human antibody.

6. The method of any one of claims 1-4, wherein the anti-IL-22 antibody, the anti-IL-6 antibody, the anti-ceacam-1 antibody, or the anti-Gr-1 antibody is a humanized antibody.

7. The method of claim 1, wherein the anti-IL-22 antibody, the anti-IL-6 antibody, the donor-type CX3CR1^{hi} MNPs, the donor-type NK cells, the ceacam-1 antagonist, or the anti-Gr-1 antibody is administered to the subject on the same day of receiving HCT.

8. The method of claim 1, wherein the anti-IL-22 antibody, the anti-IL-6 antibody, the donor-type CX3CR1^{hi} MNPs, the donor-type NK cells, the ceacam-1 antagonist, or the anti-Gr-1 antibody is administered to the subject after receiving HCT.

9. The method of any one of claims 1-8, wherein multiple doses of the anti-IL-2 antibody, the anti-IL-6 antibody, the donor-type CX3CR1^{hi} MNPs, the donor-type NK cells, the ceacam-1 antagonist, or the anti-Gr-1 antibody are administered after HCT.

10. The method of any one of claims 1-8, a single dose of the anti-IL-2 antibody, the anti-IL-6 antibody, the donor-type CX3CR1^{hi} MNPs, the donor-type NK cells, the ceacam-1 antagonist, or the anti-Gr-1 antibody is administered each day.

11. The method of any one of claims 1-10, wherein the anti-IL-2 antibody, the anti-IL-6 antibody, the donor-type CX3CR1^{hi} MNPs, the donor-type NK cells, the ceacam-1 antagonist, or the anti-Gr-1 antibody is administered every other day for a week, for two weeks, for three weeks, or for a month after HCT.

12. The method of any one of claims 1-11, wherein the subject is human.

13. The method of any one of claims 1-12, wherein the aGVHD is a gut aGVHD.

14. The method of any one of claims 1-13, wherein the aGVHD is a steroid-resistant aGVHD.

15. The method of any one of claims 1-14, wherein the anti-IL-22 antibody, the anti-IL-6 antibody, the ceacam-1 antagonist, or the anti-Gr-1 antibody is administered to the subject by oral administration or rectal administration.

16. Use an anti-IL-22 antibody, an anti-IL-6 antibody, donor-type CX3CR1^{hi} MNPs, donor-type NK cells, a ceacam-1 antagonist, an anti-Gr-1 antibody or a combination thereof for manufacturing a medicament for preventing or treating acute GVHD in a subject receiving a hematopoietic cell transplantation (HCT) or autoimmune colitis.

17. The use of claim 16, wherein the aGVHD is a gut aGVHD.

18. The use of claim 16 or claim 17, wherein the aGVHD is a steroid-resistant aGVHD.

19. A method of preventing or treating Gut-GVHD in a subject receiving a hematopoietic cell transplantation (HCT) comprising administering to the subject a ceacam-1 antagonist.

20. The method of claim 19, further comprising depleting neutrophils in the subject.

21. The method of claim 19, further comprising administering an anti-Gr-1 antibody to the subject.

22. The method of claim 21, wherein the anti-Gr-1 antibody is administered to the subject by oral administration or rectal administration

23. The method of any one of claims 19-22, wherein the ceacam-1 antagonist is administered to the subject by oral administration or rectal administration.

24. The method of any one of claims 19-23, wherein the Gut-GVHD is SR-Gut-GVHD.


SEQUENCE LISTING (12 sequences)

SEQ ID NO: 1 - DNA, 22 bp, Artificial Sequence
  Reg3gamma primer
ttcctgtcctccatgatcaaaa

SEQ ID NO: 2 - DNA, 21 bp, Artificial Sequence
  Reg3gamma primer
catccacctctgttgggttca

SEQ ID NO: 3 - DNA, 20 bp, Artificial Sequence
  Defensin-alpha3 primer
cccagaaggctcttctcttc

SEQ ID NO: 4 - DNA, 20 bp, Artificial Sequence
  Defensin-alpha3 primer
cagcgacagcagagtgtgta

SEQ ID NO: 5 - DNA, 20 bp, Artificial Sequence
  Defensin-alpha1 primer
caggccgtatctgtctcctt

SEQ ID NO: 6 - DNA, 20 bp, Artificial Sequence
  Defensin-alpha1 primer
atgaccctttctgcaggttc

SEQ ID NO: 7 - DNA, 19 bp, Artificial Sequence
  GAPDH primer
tcaccaccatggagaaggc

SEQ ID NO: 8 - DNA, 20 bp, Artificial Sequence
  GAPDH primer
gctaagcagttggtggtgca

SEQ ID NO: 9 - DNA, 22 bp, Artificial Sequence
  Actin primer
cggttggccttagggttcaggg

SEQ ID NO: 10 - DNA, 21 bp, Artificial Sequence
  Actin primer
gtgggccgctctaggcaccaa

SEQ ID NO: 11 - DNA, 47 bp, Mus musculus
acactctttccctacacgacgctcttccgatctaytgggydtaaagg

SEQ ID NO: 12 - DNA, 51 bp, Mus musculus
gtgactggagttcagacgtgtgctcttccgatctccgtcaattyhtttrgt